SEQUENCE LISTING (43 sequences)

SEQ ID NO: 1 - DNA, 3490 bp, Homo sapiens
atggtgtggtgcctcggcctggccgtcctcagcctggtcatcagccagggggctgacggtcgagggaagcctgaggtggtatcggtggtgggccgggctgaggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcagatcctggtggacgggagcctgcggctgctggccacccagcctgatgatgccggctgctacacctgtgtgcccagcaatggcctcctgcatccaccctcagcctctgcctacctcactgtgctctgcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctcctgttgtctcttggaccaaggtgggccgggggctgcaaggccaggcccaggtggacagcaacagcagcctcatcctgcgaccattgaccaaggaggcccacgggcactgggaatgcagtgccagcaatgctgtggcccgagtggccacctccacgaacgtctacgtgctgggcactaccctcatgttgtcaccaatgtgtccgtggtggctttgcccaagggtgccaatgtctcctgggagcctggctttgatggtggttatctgcagagattcagtgtctggtacaccccactggccaagcgtcctgaccgaatgcaccatgactgggtgtccttggcagtgcctgtgggggctgctcacctcctagtgccagggctgcagccccacacccagtaccagttcagcgtgctagctcagaacaagctggggagtggtcccttcagcgaaatcgtcttgtctgctccggaagggcttcctaccacgccagctgcacccgggcttcccccaacagagataccgcctcccctgtcccctccgcggggtctggtggcagtgaggacaccccggggggtactcctgcattgggatcccccagagctggtccctaagagactggatggctacgtcttggaaggccggcaaggctcccagggctgggaggtgctggacccggctgtggcaggcacagaaacagagctgctggtgccaggcctcatcaaggatgttctctacgagttccgcctcgtggccttcgcgggcagcttcgtcagcgaccccagcaacacggccaacgtctccacttccggtctggaggtctacccttcgcgcacgcagctgccgggcctcctgcctcagcccgtgctggccggcgtggtgggcggagtctgctttctgggagtggccgtccttgtgagcatcctggccggctgcctcctgaaccggcgcagggctgcccgccgccgccgcaagcgcctccgccaagatccacctcttatcttctctccgaccgggaagtcagctgcaccctctgctctgggctcaggcagtcctgacagcgtggcgaagctgaagctccagggatccccagtccccagcctgcgccagagtctgctctggggggatcctgccggaactcccagcccccacccggatcctccatctagccggggacccttacctctggagcccatttgccggggcccagacgggcgctttgtgatggggcccactgtggcggccccccaggaaaggtcaggccgggagcaggcagaacctcggactccagcccagcgtctggcccggtcctttgactgtagcagcagcagccccagtggggcaccccagcccctctgcattgaagacatcagccctgtggcaccccctccagcagccccacccagtcccttgccaggtcctggacccctgctccagtacctgagcctgcccttcttccgagagatgaatgtggatggggactggcccccgcttgaggagcccagccctgctgcacccccagattacatggatacccggcgctgtcccacctcatctttccttcgttctccagaaacccctcctgtatcccccagggaatcacttcctggggctgtggtaggggctggggccactgcagagcccccttacaagccctggctgactggacactgagggagcggctgctgccaggccttctccctgctgcccctcgaggcagcctcaccagccagagcagcgggcgaggcagcgcttcgttcctgcggcccccctccacagccccctctgcaggaggcagctacctcagccctgctccaggagacaccagcagctgggccagtggccctgagagatggccccgaagggagcatgtggtgacagtcagcaagaggaggaacacatctgtggacgagaactatgagtgggactcagaattccctggggacatggaattgctggagactttgcacctgggcttggccagctcccggctcagacctgaagctgagacagagctaggtgtgaagactccagaggagggctgcctcctgaacactgcccatgttactggccctgaggcccgctgtgctgcccttcgggaggaattcctggccttccgccgccgccgagatgctactagggctcggctaccagcctatcgacagccagtcccccaccccgaacaggccactctgctgtga

SEQ ID NO: 2 - protein, 1163 aa, Homo sapiens
MVWCLGLAVLSLVISQGADGRGKPEVVSVVGRAEESVVLGCDLLPPAGRPPLHVIEWLRFGFLLPIFIQFGLYSPRIDPDYVGRVRLQKGASLQIEGLRVEDQGWYECRVFFLDQHIPEDDFANGSWVHLTVNSPPQFQETPPAVLEVQELEPVTLRCVARGSPLPHVTWKLRGKDLGQGQGQVQVQNGTLRIRRVERGSSGVYTCQASSTEGSATHATQLLVLGPPVIVVPPKNSTVNASQDVSLACHAEAYPANLTYSWFQDNINVFHISRLQPRVQILVDGSLRLLATQPDDAGCYTCVPSNGLLHPPSASAYLTVLCMPGVIRCPVRANPPLLFVSWTKDGKALQLDKFPGWSQGTEGSLIIALGNEDALGEYSCTPYNSLGTAGPSPVTRVLLKAPPAFIERPKEEYFQEVGRELLIPCSAQGDPPPVVSWTKVGRGLQGQAQVDSNSSLILRPLTKEAHGHWECSASNAVARVATSTNVYVLGTSPHVVTNVSVVALPKGANVSWEPGFDGGYLQRFSVWYTPLAKRPDRMHHDWVSLAVPVGAAHLLVPGLQPHTQYQFSVLAQNKLGSGPFSEIVLSAPEGLPTTPAAPGLPPTEIPPPLSPPRGLVAVRTPRGVLLHWDPPELVPKRLDGYVLEGRQGSQGWEVLDPAVAGTETELLVPGLIKDVLYEFRLVAFAGSFVSDPSNTANVSTSGLEVYPSRTQLPGLLPQPVLAGVVGGVCFLGVAVLVSILAGCLLNRRRAARRRRKRLRQDPPLIFSPTGKSAAPSALGSGSPDSVAKLKLQGSPVPSLRQSLLWGDPAGTPSPHPDPPSSRGPLPLEPICRGPDGRFVMGPTVAAPQERSGREQAEPRTPAQRLARSFDCSSSSPSGAPQPLCIEDISPVAPPPAAPPSPLPGPGPLLQYLSLPFFREMNVDGDWPPLEEPSPAAPPDYMDTRRCPTSSFLRSPETPPVSPRESLPGAVVGAGATAEPPYTALADWTLRERLLPGLLPAAPRGSLTSQSSGRGSASFLRPPSTAPSAGGSYLSPAPGDTSSWASGPERWPRREHVVTVSKRRNTSVDENYEWDSEFPGDMELLETLHLGLASSRLRPEAETELGVKTPEEGCLLNTAHVTGPEARCAALREEFLAFRRRRDATRARLPAYRQPVPHPEQATLL

SEQ ID NO: 3 - DNA, 2862 bp, Homo sapiens
atggtgtggtgcctcggcctggccgtcctcagcctggtcatcagccagggggctgacggtcgagggaagcctgaggtggtatcggtggtgggccgggctgaggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcagatcctggtggacgggagcctgcggctgctggccacccagcctgatgatgccggctgctacacctgtgtgcccagcaatggcctcctgcatccaccctcagcctctgcctacctcactgtgctctgcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctcctgttgtctcttggaccaaggtgggccgggggctgcaaggccaggcccaggtggacagcaacagcagcctcatcctgcgaccattgaccaaggaggcccacgggcactgggaatgcagtgccagcaatgctgtggcccgagtggccacctccacgaacgtctacgtgctgggcactagccctcatgttgtcaccaatgtgtccgtggtggctttgcccaagggtgccaatgtctcctgggagcctggctttgatggtggttatctgcagagattcagtgtctggtacaccccactggccaagcgtcctgaccgaatgcaccatgactgggtgtccttggcagtgcctgtgggggctgctcacctcctagtgccagggctgcagccccacacccagtaccagttcagcgtgctagctcagaacaagctggggagtggtcccttcagcgaaatcgtcttgtctgctccggaagggcttcctaccacgccagctgcacccgggcttcccccaacagagataccgcctcccctgtcccctccgcggggtctggtggcagtgaggacaccccggggggtactcctgcattgggatcccccagagctggtccctaagagactggatggctacgtcttggaaggccggcaaggctcccagggctgggaggtgctggacccggctgtggcaggcacagaaacagagctgctggtgccaggcctcatcaaggatgttctctacgagttccgcctcgtggccttcgcgggcagcttcgtcagcgaccccagcaacacggccaacgtctccacttccggtctggaggtctacccttcgcgcacgcagctgccgggcctcctgcctcagccctctagccaggagcccaaaagctctgacaaaactcacacatccccaccgtccccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 4 - protein, 953 aa, Homo sapiens
MVWCLGLAVLSLVISQGADGRGKPEVVSVVGRAEESVVLGCDLLPPAGRPPLHVIEWLRFGFLLPIFIQFGLYSPRIDPDYVGRVRLQKGASLQIEGLRVEDQGWYECRVFFLDQHIPEDDFANGSWVHLTVNSPPQFQETPPAVLEVQELEPVTLRCVARGSPLPHVTWKLRGKDLGQGQGQVQVQNGTLRIRRVERGSSGVYTCQASSTEGSATHATQLLVLGPPVIVVPPKNSTVNASQDVSLACHAEAYPANLTYSWFQDNINVFHISRLQPRVQILVDGSLRLLATQPDDAGCYTCVPSNGLLHPPSASAYLTVLCMPGVIRCPVRANPPLLFVSWTKDGKALQLDKFPGWSQGTEGSLIIALGNEDALGEYSCTPYNSLGTAGPSPVTRVLLKAPPAFIERPKEEYFQEVGRELLIPCSAQGDPPPVVSWTKVGRGLQGQAQVDSNSSLILRPLTKEAHGHWECSASNAVARVATSTNVYVLGTSPHVVTNVSVVALPKGANVSWEPGFDGGYLQRFSVWYTPLAKRPDRMHHDWVSLAVPVGAAHLLVPGLQPHTQYQFSVLAQNKLGSGPFSEIVLSAPEGLPTTPAAPGLPPTEIPPPLSPPRGLVAVRTPRGVLLHWDPPELVPKRLDGYVLEGRQGSQGWEVLDPAVAGTETELLVPGLIKDVLYEFRLVAFAGSFVSDPSNTANVSTSGLEVYPSRTQLPGLLPQPSSQEPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 2910 bp, Homo sapiens
atggtgtggtgcctcggcctggccgtcctcagcctggtcatcagccagggggctgacggtcgagggaagcctgaggtggtatcggtggtgggccgggctgaggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcagatcctggtggacgggagcctgcggctgctggccacccagcctgatgatgccggctgctacacctgtgtgcccagcaatggcctcctgcatccaccctcagcctctgcctacctcactgtgctctacccagcccaggtgacagctatgcctcctgagacacccctgcccataggcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctcctgttgtctcttggaccaaggtgggccgggggctgcaaggccaggcccaggtggacagcaacagcagcctcatcctgcgaccattgaccaaggaggcccacgggcactgggaatgcagtgccagcaatgctgtggcccgagtggccacctccacgaacgtctacgtgctgggcactagccctcatgttgtcaccaatgtgtccgtggtggctttgcccaagggtgccaatgtctcctgggagcctggctttgatggtggttatctgcagagattcagtgtctggtacaccccactggccaagcgtcctgaccgaatgcaccatgactgggtgtccttggcagtgcctgtgggggctgctcacctcctagtgccagggctgcagccccacacccagtaccagttcagcgtgctagctcagaacaagctggggagtggtcccttcagcgaaatcgtcttgtctgctccggaagggcttcctaccacgccagctgcacccgggcttcccccaacagagataccgcctcccctgtcccctccgcggggtctggtggcagtgaggacaccccggggggtactcctgcattgggatcccccagagctggtccctaagagactggatggctacgtcttggaaggccggcaaggctcccagggctgggaggtgctggacccggctgtggcaggcacagaaacagagctgctggtgccaggcctcatcaaggatgttctctacgagttccgcctcgtggccttcgcgggcagcttcgtcagcgaccccagcaacacggccaacgtctccacttccggtctggaggtctacccttcgcgcacgcagctgccgggcctcctgcctcagccctctagccaggagcccaaaagctctgacaaaactcacacatccccaccgtccccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 6 - protein, 969 aa, Homo sapiens [sequence omitted]

SEQ ID NO: 7 - DNA, 3540 bp, Homo sapiens
atggtgtggtgcctcggcctggccgtcctcagcctggtcatcagccagggggctgacggtcgagggaagcctgaggtggtatcggtggtgggccgggctggggagagtgtggtgctgggctgtgacctgctgcccccggccggccggccccccctgcatgtcatcgagtggctgcgctttggattcctgcttcccatcttcatccagttcggcctctactctccccgaattgaccctgattacgtgggacgagtccggctgcagaagggggcctctctccagattgagggtctccgggtggaagaccagggctggtacgagtgccgcgtgttcttcctggaccagcacatccctgaagacgattttgctaacggctcctgggtgcatctgacagtcaattcaccccctcaattccaggagacacctcctgctgtgttggaagtgcaggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctctacccagcccaggtgacagctatgcctcctgagacacccctgcccataggcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagagcggcccaaggaagaatatttccaagaagtagggcgggagctgctcatcccctgctccgcccaaggggaccctcctcctgttgtctcttggaccaaggtgggccgggggctgcaaggccaggcccaggtggacagcaacagcagcctcatcctgcgaccattgaccaaggaggcccacgggcactgggaatgcagtgccagcaatgctgtggcccgagtggccacctccacgaacgtctacgtgctgggcactagccctcatgttgtcaccaatgtgtccgtggtggctttgcccaagggtgccaatgtctcctgggagcctggctttgatggtggttatctgcagagattcagtgtctggtacaccccactggccaagcgtcctgaccgaatgcaccatgactgggtgtccttggcagtgcctgtgggggctgctcacctcctagtgccagggctgcagccccacacccagtaccagttcagcgtgctagctcagaacaagctggggagtggtcccttcagcgaaatcgtcttgtctgctccggaagggcttcctaccacgccagctgcacccgggcttcccccaacagagataccgcctcccctgtcccctccgcggggtctggtggcagtgaggacaccccggggggtactcctgcattgggatcccccagagctggtccctaagagactggatggctacgtcttggaaggccggcaaggctcccagggctgggaggtgctggacccggctgtggcaggcacagaaacagagctgctggtgccaggcctcatcaaggatgttctctacgagttccgcctcgtggccttcgcgggcagcttcgtcagcgaccccagcaacacggccaacgtctccacttccggtctggaggtctacccttcgcgcacgcagctgccgggcctcctgcctcagcccgtgctggccggcgtggtgggcggagtctgctttctgggagtggccgtccttgtgagcatcctggccggctgcctcctgaaccggcgcagggctgcccgccgccgccgcaagcgcctccgccaagatccacctcttatcttctctccgaccgggaagtcagctgcaccctctgctctgggctcaggcagtcctgacagcgtggcgaagctgaagctccagggatccccagtccccagcctgcgccagagtctgctctggggggatcctgccggaactcccagcccccacccggatcctccatctagccggggacccttacctctggagcccatttgccggggcccagacgggcgctttgtgatggggcccactgtggcggccccccaggaaaggtcaggccgggagcaggcagaacctcggactccagcccagcgtctggcccggtcctttgactgtagcagcagcagccccagtggggcaccccagcccctctgcattgaagacatcagccctgtggcaccccctccagcagccccacccagtcccttgccaggtcctggacccctgctccagtacctgagcctgcccttcttccgagagatgaatgtggatggggactggcccccgcttgaggagcccagccctgctgcacccccagattacatggatacccggcgctgtcccacctcatctttccttcgttctccagaaacccctcctgtatcccccagggaatcacttcctggggctgtggtaggggctggggccactgcagagcccccttacacagccctggctgactggacactgagggagcggctgctgccaggccttctccctgctgcccctcgaggcagcctcaccagccagagcagtgggcgaggcagcgcttcgttcctgcggcccccctccacagccccctctgcaggaggcagctacctcagccctgctccaggagacaccagcagctgggccagtggccctgagagatggccccgaagggagcatgtggtgacagtcagcaagaggaggaacacatctgtggacgagaactatgagtgggactcagaattccctggggacatggaattgctggagactttgcacctgggcttggccagctcccggctcagacctgaagctgagccagagctaggtgtgaagactccagaggagggctgcctcctgaacactgcccatgttactggccctgaggcccgctgtgctgcccttcgggaggaattcctggccttccgccgccgccgagatgctactagggctcggctaccagcctatcgacagccagtcccccaccccgaacaggccactctgctgtga

SEQ ID NO: 8 - protein, 1179 aa, Homo sapiens
MVWCLGLAVLSLVISQGADGRGKPEVVSVVGRAGESVVLGCDLLPPAGRPPLHVIEWLRFGFLLPIFIQFGLYSPRIDPDYVGRVRLQKGASLQIEGLRVEDQGWYECRVFFLDQHIPEDDFANGSWVHLTVNSPPQFQETPPAVLEVQELEPVTLRCVARGSPLPHVTWKLRGKDLGQGQGQVQVQNGTLRIRRVERGSSGVYTCQASSTEGSATHATQLLVLGPPVIVVPPKNSTVNASQDVSLACHAEAYPANLTYSWFQDNINVFHISRLQPRVRILVDGSLRLLATQPDDAGCYTCVPSNGLLHPPSASAYLTVLYPAQVTAMPPETPLPIGMPGVIRCPVRANPPLLFVSWTKDGKALQLDKFPGWSQGTEGSLIIALGNEDALGEYSCTPYNSLGTAGPSPVTRVLLKAPPAFIERPKEEYFQEVGRELLIPCSAQGDPPPVVSWTKVGRGLQGQAQVDSNSSLILRPLTKEAHGHWECSASNAVARVATSTNVYVLGTSPHVVTNVSVVALPKGANVSWEPGFDGGYLQRFSVWYTPLAKRPDRMHHDWVSLAVPVGAAHLLVPGLQPHTQYQFSVLAQNKLGSGPFSEIVLSAPEGLPTTPAAPGLPPTEIPPPLSPPRGLVAVRTPRGVLLHWDPPELVPKRLDGYVLEGRQGSQGWEVLDPAVAGTETELLVPGLIKDVLYEFRLVAFAGSFVSDPSNTANVSTSGLEVYPSRTQLPGLLPQPVLAGVVGGVCFLGVAVLVSILAGCLLNRRRAARRRRKRLRQDPPLIFSPTGKSAAPSALGSGSPDSVAKLKLQGSPVPSLRQSLLWGDPAGTPSPHPDPPSSRGPLPLEPICRGPDGRFVMGPTVAAPQERSGREQAEPRTPAQRLARSFDCSSSSPSGAPQPLCIEDISPVAPPPAAPPSPLPGPGPLLQYLSLPFFREMNVDGDWPPLEEPSPAAPPDYMDTRRCPTSSFLRSPETPPVSPRESLPGAVVGAGATAEPPYTALADWTLRERLLPGLLPAAPRGSLTSQSSGRGSASFLRPPSTAPSAGGSYLSPAPGDTSSWASGPERWPRREHVVTVSKRRNTSVDENYEWDSEFPGDMELLETLHLGLASSRLRPEAEPELGVKTPEEGCLLNTAHVTGPEARCAALREEFLAFRRRRDATRARLPAYRQPVPHPEQATLL

SEQ ID NO: 9 - protein, 1179 aa, Homo sapiens
MVWCLGLAVLSLVISQGADGRGKPEVVSVVGRAGESVVLGCDLLPPAGRPPLHVIEWLRFGFLLPIFIQFGLYSPRIDPDYVGRVRLQKGASLQIEGLRVEDQGWYECRVFFLDQHIPEDDFANGSWVHLTVNSPPQFQETPPAVLEVQELEPVTLRCVARGSPLPHVTWKLRGKDLGQGQGQVQVQNGTLRIRRVERGSSGVYTCQASSTEGSATHATQLLVLGPPVIVVPPKNSTVNASQDVSLACHAEAYPANLTYSWFQDNINVFHISRLQPRVRILVDGSLRLLATQPDDAGCYTCVPSNGLLHPPSASAYLTVLYPAQVTAMPPETPLPIGMPGVIRCPVRANPPLLFVSWTKDGKALQLDKFPGWSQGTEGSLIIALGNEDALGEYSCTPYNSLGTAGPSPVTRVLLKAPPAFIERPKEEYFQEVGRELLIPCSAQGDPPPVVSWTKVGRGLQGQAQVDSNSSLILRPLTKEAHGHWECSASNAVARVATSTNVYVLGTSPHVVTNVSVVALPKGANVSWEPGFDGGYLQRFSVWYTPLAKRPDRMHHDWVSLAVPVGAAHLLVPGLQPHTQYQFSVLAQNKLGSGPFSEIVLSAPEGLPTTPAAPGLPPTEIPPPLSPPRGLVAVRTPRGVLLHWDPPELVPKRLDGYVLEGRQGSQGWEVLDPAVAGTETELLVPGLIKDVLYEFRLVAFAGSFVSDPSNTANVSTSGLEVYPSRTQLPGLLPQPVLAGVVGGVCFLGVAVLVSILAGCLLNRRRAARRRRKRLRQDPPLIFSPTGKSAAPSALGSGSPDSVAKLKLQGSPVPSLRQSLLWGDPAGTPSPHPDPPSSRGPLPLEPICRGPDGRFVMGPYVAAPQERSGREQAEPRTPAQRLARSFDCSSSSPSGAPQPLCIEDISPVAPPPAAPPSPLPGPGPLLQYLSLPFFREMNVDGDWPPLEEPSPAAPPDYMDTRRCPTSSFLRSPETPPVSPRESLPGAVVGAGATAEPPYTALADWTLRERLLPGLLPAAPRGSLTSQSSGRGSASFLRPPSTAPSAGGSYLSPAPGDTSSWASGPERWPRREHVVTVSKRRNTSVDENYEWDSEFPGDMELLETLHLGLASSRLRPEAEPELGVKTPEEGCLLNTAHVTGPEARCAALREEFLAFRRRRDATRARLPAYRQPVPHPEQATLL

SEQ ID NO: 10 - protein, 1163 aa, Homo sapiens
MVWCLGLAVLSLVISQGADGRGKPEVVSVVGRAEESVVLGCDLLPPAGRPPLHVIEWLRFGFLLPIFIQFGLYSPRIDPDYVGRVRLQKGASLQIEGLRVEDQGWYECRVFFLDQHIPEDDFANGSWVHLTVNSPPQFQETPPAVLEVQELEPVTLRCVARGSPLPHVTWKLRGKDLGQGQGQVQVQNGTLRIRRVERGSSGVYTCQASSTEGSATHATQLLVLGPPVIVVPPKNSTVNASQDVSLACHAEAYPANLTYSWFQDNINVFHISRLQPRVQILVDGSLRLLATQPDDAGCYTCVPSNGLLHPPSASAYLTVLCMPGVIRCPVRANPPLLFVSWTKDGKALQLDKFPGWSQGTEGSLIIALGNEDALGEYSCTPYNSLGTAGPSPVTRVLLKAPPAFIERPKEEYFQEVGRELLIPCSAQGDPPPVVSWTKVGRGLQGQAQVDSNSSLILRPLTKEAHGHWECSASNAVARVATSTNVYVLGTSPHVVTNVSVVALPKGANVSWEPGFDGGYLQRFSVWYTPLAKRPDRMHHDWVSLAVPVGAAHLLVPGLQPHTQYQFSVLAQNKLGSGPFSEIVLSAPEGLPTTPAAPGLPPTEIPPPLSPPRGLVAVRTPRGVLLHWDPPELVPKRLDGYVLEGRQGSQGWEVLDPAVAGTETELLVPGLIKDVLYEFRLVAFAGSFVSDPSNTANVSTSGLEVYPSRTQLPGLLPQPVLAGVVGGVCFLGVAVLVSILAGCLLNRRRAARRRRKRLRQDPPLIFSPTGKSAAPSALGSGSPDSVAKLKLQGSPVPSLRQSLLWGDPAGTPSPHPDPPSSRGPLPLEPICRGPDGRFVMGPYVAAPQERSGREQAEPRTPAQRLARSFDCSSSSPSGAPQPLCIEDISPVAPPPAAPPSPLPGPGPLLQYLSLPFFREMNVDGDWPPLEEPSPAAPPDYMDTRRCPTSSFLRSPETPPVSPRESLPGAVVGAGATAEPPYTALADWTLRERLLPGLLPAAPRGSLTSQSSGRGSASFLRPPSTAPSAGGSYLSPAPGDTSSWASGPERWPRREHVVTVSKRRNTSVDENYEWDSEFPGDMELLETLHLGLASSRLRPEAETELGVKTPEEGCLLNTAHVTGPEARCAALREEFLAFRRRRDATRARLPAYRQPVPHPEQATLL

SEQ ID NO: 11 - protein, 1163 aa, Homo sapiens
MVWCLGLAVLSLVISQGADGRGKPEVVSVVGRAEESVVLGCDLLPPAGRPPLHVIEWLRFGFLLPIFIQFGLYSPRIDPDYVGRVRLQKGASLQIEGLRVEDQGWYECRVFFLDQHIPEDDFANGSWVHLTVNSPPQFQETPPAVLEVQELEPVTLRCVARGSPLPHVTWKLRGKDLGQGQGQVQVQNGTLRIRRVERGSSGVYTCQASSTEGSATHATQLLVLGPPVIVVPPKNSTVNASQDVSLACHAEAYPANLTYSWFQDNINVFHISRLQPRVRILVDGSLRLLATQPDDAGCYTCVPSNGLLHPPSASAYLTVLCMPGVIRCPVRANPPLLFVSWTKDGKALQLDKFPGWSQGTEGSLIIALGNEDALGEYSCTPYNSLGTAGPSPVTRVLLKAPPAFIERPKEEYFQEVGRELLIPCSAQGDPPPVVSWTKVGRGLQGQAQVDSNSSLILRPLTKEAHGHWECSASNAVARVATSTNVYVLGTSPHVVTNVSVVALPKGANVSWEPGFDGGYLQRFSVWYTPLAKRPDRMHHDWVSLAVPVGAAHLLVPGLQPHTQYQFSVLAQNKLGSGPFSEIVLSAPEGLPTTPAAPGLPPTEIPPPLSPPRGLVAVRTPRGVLLHWDPPELVPKRLDGYVLEGRQGSQGWEVLDPAVAGTETELLVPGLIKDVLYEFRLVAFAGSFVSDPSNTANVSTSGLEVYPSRTQLPGLLPQPVLAGVVGGVCFLGVAVLVSILAGCLLNRRRAARRRRKRLRQDPPLIFSPTGKSAAPSALGSGSPDSVAKLKLQGSPVPSLRQSLLWGDPAGTPSPHPDPPSSRGPLPLEPICRGPDGRFVMGPYVAAPQERSGREQAEPRTPAQRLARSFDCSSSSPSGAPQPLCIEDISPVAPPPAAPPSPLPGPGPLLQYLSLPFFREMNVDGDWPPLEEPSPAAPPDYMDTRRCPTSSFLRSPETPPVSPRESLPGAVVGAGATAEPPYTALADWTLRERLLPGLLPAAPRGSLTSQSSGRGSASFLRPPSTAPSAGGSYLSPAPGDTSSWASGPERWPRREHVVTVSKRRNTSVDENYEWDSEFPGDMELLETLHLGLASSRLRPEAEPELGVKTPEEGCLLNTAHVTGPEARCAALREEFLAFRRRRDATRARLPAYRQPVPHPEQATLL

SEQ ID NO: 12 - DNA, 1030 bp, Homo sapiens
caggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctctgtaagcctgacctcagcttctccctcagcctgctcccttcccctgggccaggccaagcccctctcccccaacttgccactattttcccccagacccagcccaggtgacagctatgcctcctgagacacccctgcccataggcatgccgggggtgatccgctgcccggttcgtgccaaccccccactgctctttgtcagctggaccaaggatggaaaggccctgcagctggacaagaagagagatgatctctggggaaaatgatggcaaagagtcaagaaggagaactgaagtttctttcgtgtgatgactgggaaattgtgtgtcccgggggaatacacacttcttaccagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagag

SEQ ID NO: 13 - DNA, 939 bp, Homo sapiens
caggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctctacccagcccaggtgacagctatgcctcctgagacacccctgcccataggcatgccgggggtgatccgctgcccggttcgtgccaaccccccactgctctttgtcagctggaccaaggatggaaaggccctgcagctggacaagaagagagatgatctccggggaaaatgatggcaaagagtcaagaaggagaactgaagtttctttcgtgtgatgactgggaaattgtgtgtcccgggggaacacacacttcttaccagttccctggctggtcccagggcacagaaggctcactgatcatcgccctggggaacgaggatgccctgggagaatactcctgcacccccctacaacagtcttggtaccgccgggccctctcctgtgacccgcgtgctgctcaaggctcccccagcttttatagag

SEQ ID NO: 14 - DNA, 832 bp, Homo sapiens
caggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctctctggaccaaggatggaaaggccctgcagctggacaagaagagagatgatctctggggaaaatgatggcaaagagtcaagaaggagaactgaagtttctttcgtgtgatgactgggaaattgtgtgtcccgggggaacacacacttcttaccagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagag

SEQ ID NO: 15 - DNA, 822 bp, Homo sapiens
caggaactggagcctgtgaccctgcgttgtgtggcccgtggcagccccctgcctcatgtgacgtggaagctccgaggaaaggaccttggccagggccagggccaggtgcaagtgcagaacgggacgctgcggatccgccgggtagagcgaggcagctctggggtctacacctgccaagcctccagcactgagggcagcgccacccacgccacccagctgctagtgctaggacccccagtcatcgtggtgccccccaagaacagcacagtcaatgcctcccaggatgtttcattggcctgccatgctgaggcataccctgctaacctcacctacagctggttccaggacaacatcaatgtcttccacattagccgcctgcagccccgggtgcggatcctggtggacgggagcctgcggctgctggccacccagcctgatgatgccggctgctacacctgtgtgcccagcaatggcctcctgcatccaccctcagcctctgcctacctcactgtgctctacccagcccaggtgacagctatgcctcctgagacacccctgcccataggcatgccgggggtgatccgctgcccggttcgtgccaaccccccactgctctttgtcagctggaccaaggatggaaaggccctgcagctggacaagttccctggctggtcccagggcacagaaggctcactgatcatcgccctggggaacgaggatgccctgggagaatactcctgcaccccctacaacagtcttggtaccgccgggccctctcctgtgacccgcgtgctgctcaaggctcccccagcttttatagag

SEQ ID NO: 16 - DNA, 222 bp, Homo sapiens
ctctgcctacctcactgtgctctacccagcccaggtgacagctatgcctcctgagacacccctgcccataggcatgccgggggtgatccgctgcccggttcgtgccaaccccccactgctctttgtcagctggaccaaggatggaaaggccctgcagctggacaagttccctggctggctggtcccagggcacagaaggctcactgatcatcgccctgggga

SEQ ID NO: 17 - DNA, 222 bp, Homo sapiens; identical to SEQ ID NO: 16

SEQ ID NO: 18 - DNA, 226 bp, Homo sapiens
ctctgcctacctcactgtgctctctggaccaaggatggaaaggccctgcagctggacaagaagagagatgatctggggaaaatgatggcaaagagtcaagaaggagaactgaagtttctttcgtgtgatgactgggaaattgtgtgtcccgggggaacacacacttcttaccagttccctggctggtcccagggcacagaaggctcactgatcatcgccctgggga

SEQ ID NO: 19 - DNA, 426 bp, Homo sapiens
ctctgcctacctcactgtgctctgtaagcctgacctcagcttctccctcagcctgctcccttcccctgggccaggccaagcccctctcccccaacttgccactattttcccccagacccagcccaggtgacagctatgcctcctgagacacccctgcccataggcatgccgggggtgatccgctgcccggttcgtgccaaccccccactgctctttgtcagctggaccaaggatggaaaggccctgcagctggacaagaagagagatgatctctggggaaaatgatggcaaagagtcaagaaggagaactgaagtttctttcgtgtgatgactgggaaattgtgtgtcccgggggaacacacacttcttaccagttccctggctggtcccagggcacagaaggctcactgatcatcgccctgggga

SEQ ID NO: 20 - DNA, 336 bp, Homo sapiens
ctctgcctacctcactgtgctctacccagcccaggtgacagctatgcctcctgagacacccctgcccataggcatgccgggggtgatccgctgcccggttcgtgccaaccccccactgctctttgtcagctggaccaaggatggaaaggccctgcagctggacaagaagagagatgatctccggggaaaatgatggcaaagagtcaagaaggagaactgaagtttctttcgtgtgtgatgactgggaaattgtgtgtcccgggggaacacacacttcttaccagttccctggctggtcccagggcacagaaggctcactgatcatcgccctgggga

SEQ ID NO: 21 - DNA, 332 bp, Homo sapiens
ctctgcctacctcactgtgctctacccagcccaggtgacagctatgcctcctgagacacccctgcccataggcatgccgggggtgatccgctgcccggttcgtgccaaccccccactgctctttgtcagctggaccaaggatggaaaggccctgcagctggacaagaagagagatgatctcggggaaaatgatggcaaagagtcaagaaggagaactgaagtttctttcgtgtgatgactgggaaattgtgtgtcccgggggaaacacacttcttaccagttccctggctggtcccagggcacagaaggctcactgatcatcgccctgggga

SEQ ID NO: 22 - protein, 141 aa, Homo sapiens
SLRLLATQPDDAGCYTCPSNGLLHPPSASAYLTVLYPAQVTAMPPETPLPIGMPGVIRCPVRANPPLLFVSWTKDGKALQLDKFPGWSQGTEGSLIIALGNEDALGEYSCTPYNSLGTAGPSPVTRVLLKAPPAFIERPKE

SEQ ID NO: 23 - protein, 142 aa, Homo sapiens
SLRLLATQPDDAGCYTCVPSNGLLHPPSASAYLTVLYPAQVTAMPPETPLPIGMPGVIRCPVRANPPLLFVSWTKDGKALQLDKFPGWSQGTEGSLIIALGNEDALGEYSCTPYNSLGTAGPSPVTRVLLKAPPAFIERPKE

SEQ ID NO: 24 - protein, 71 aa, Homo sapiens
SLRLLATQPDDAGCYTCVPSNGLLHPPSASAYLTVLSGPRMERPCSWTRREMISGENDGKESRRRTEVSFV

SEQ ID NO: 25 - protein, 71 aa, Homo sapiens
SLRLLATQPDDAGCYTCVPSNGLLHPPSASAYLTVLCKPDLSFSLSLLPSPGPGQAPLPQLATIFPQTQPR

SEQ ID NO: 26 - protein, 100 aa, Homo sapiens
SLRLLATQPDDAGCYTCVPSNGLLHPPSASAYLTVLYPAQVTAMPPETPLPIGMPGVIRCPVRANPPLLFVSWTKDGKALQLDKKRDDLRGKWQRVKKEN

SEQ ID NO: 27 - protein, 86 aa, Homo sapiens
SLRLLATQPDDAGCYTCVPSNGLLHPPSASAYLTVLYPAQVTAMPPETPLPIGMPGVIRCPVRANPPLLFVSWTKDGKALQLDKGI

SEQ ID NO: 28 - DNA, 2250 bp, Homo sapiens
atggcgaggaagttatctgtaatcttgatcctgacctttgccctctctgtcacaaatccccttcatgaactaaaagcagctgctttcccccagaccactgagaaaattagtccgaattgggaatctggcattaatgttgacttggcaatttccacacggcaatatcatctacaacagcttttctaccgctatggagaaaataattctttgtcagttgaagggttcagaaaattacttcaaaatataggcatagataagattaaaagaatccatatacaccatgaccacgaccatcactcagaccacgagcatcactcagaccatgagcgtcactcagaccatgagcatcactcagaccacgagcatcactctgaccataatcatgctgcttctggtaaaaataagcgaaaagctctttgcccagaccatgactcagatagttcaggtaaagatcctagaaacagccaggggaaaggagctcaccgaccagaacatgccagtggtagaaggaatgtcaaggacagtgttagtgctagtgaagtgacctcaactgtgtacaacactgtctctgaaggaactcactttctagagacaatagagactccaagacctggaaaactcttccccaaagatgtaagcagctccactccacccagtgtcacatcaaagagccgggtgagccggctggctggtaggaaaacaaatgaatctgtgagtgagccccgaaaaggctttatgtattccagaaacacaaatgaaaatcctcaggagtgtttcaatgcatcaaagctactgacatctcatggcatgggcatccaggttccgctgaatgcaacagagttcaactatctctgtccagccatcatcaaccaaattgatgctagatcttgtctgattcatacaagtgaaaagaaggctgaaatccctccaaagacctattcattacaaatagcctgggttggtggttttatagccatttccatcatcagtttcctgtctctgctgggggttatcttagtgcctctcatgaatcgggtgtttttcaaatttctcctgagtttccttgtggcactggccgttgggactttgagtggtgatgcttttttacaccttcttccacattctcatgcaagtcaccaccatagtcatagccatgaagaaccagcaatggaaatgaaaagaggaccacttttcagtcatctgtcttctcaaaacatagaagaaagtgcctattttgattccacgtggaagggtctaacagctctaggaggcctgtatttcatgtttcttgttgaacatgtcctcacattgatcaaacaatttaaagataagaagaaaaagaatcagaagaaacctgaaaatgatgatgatgtggagattaagaagcagttgtccaagtatgaatctcaactttcaacaaatgaggagaaagtagatacagatgatcgaactgaaggctatttacgagcagactcacaagagccctcccactttgattctcagcagcctgcagtcttggaagaagaagaggtcatgatagctcatgctcatccacaggaagtctacaatgaatatgtacccagagggtgcaagaataaatgccattcacatttccacgatacactcggccagtcagacgatctcattcaccaccatcatgactaccatcatattctccatcatcaccaccaccaaaaccaccatcctcacagtcacagccagcgctactctcgggaggagctgaaagatgccggcgtcgccactttggcctggatggtgataatgggtgatggcctgcacaatttcagcgatggcctagcaattggtgctgcttttactgaaggcttatcaagtggtttaagtacttctgttgctgtgttctgtcatgagttgcctcatgaattaggtgactttgctgttctactaaaggctggcatgaccgttaagcaggctgtcctttataatgcattgtcagccatgctggcgtatcttggaatggcaacaggaattttcattggtcattatgctgaaaatgtttctatgtggatatttgcacttactgctggcttattcatgtatgttgctctggttgatatggtacctgaaatgctgcacaatgatgctagtgaccatggatgtagccgctgggggtatttctttttacagaatgctgggatgcttttgggttttggaattatgttacttatttccatatttgaacataaaatcgtgtttcgtataaatttctag

SEQ ID NO: 29 - protein, 749 aa, Homo sapiens
MARKLSVILILTFALSVTNPLHELKAAAFPQTTEKISPNWESGINVDLAISTRQYHLQQLFYRYGENNSLSVEGFRKLLQNIGIDKIKRIHIHHDHDHHSDHEHHSDHERHSDHEHHSDHEHHSDHNHAASGKNKRKALCPDHDSDSSGKDPRNSQGKGAHRPEHASGRRNVKDSVSASEVTSTVYNTVSEGTHFLETIETPRPGKLFPKDVSSSTPPSVTSKSRVSRLAGRKTNESVSEPRKGFMYSRNTNENPQECFNASKLLTSHGMGIQVPLNATEFNYLCPAIINQIDARSCLIHTSEKKAEIPPKTYSLQIAWVGGFIAISIISFLSLLGVILVPLMNRVFFKFLLSFLVALAVGTLSGDAFLHLLPHSHASHHHSHSHEEPAMEMKRGPLFSHLSSQNIEESAYFDSTWKGLTALGGLYFMFLVEHVLTLIKQFKDKKKKNQKKPENDDDVEIKKQLSKYESQLSTNEEKVDTDDRTEGYLRADSQEPSHFDSQQPAVLEEEEVMIAHAHPQEVYNEYVPRGCKNKCHSHFHDTLGQSDDLIHHHHDYHHILHHHHHQNHHPHSHSQRYSREELKDAGVATLAWMVIMGDGLHNFSDGLAIGAAFTEGLSSGLSTSVAVFCHELPHELGDFAVLLKAGMTVKQAVLYNALSAMLAYLGMATGIFIGHYAENVSMWIFALTAGLFMYVALVDMVPEMLHNDASDHGCSRWGYFFLQNAGMLLGFGIMLLISIFEHKIVFRINF

SEQ ID NO: 30 - DNA, 25 bp, Artificial Sequence
  Primer used to amplify IGSF9
tcttatcttctctccgaccgggaag

SEQ ID NO: 31 - DNA, 25 bp, Artificial Sequence
  Primer used to amplify IGSF9
gccacagggctgatgtcttcaatgc

SEQ ID NO: 32 - DNA, 20 bp, Artificial Sequence
  Primer used for amplification of the GAPDH gene
accacagtccatgccatcac

SEQ ID NO: 33 - DNA, 20 bp, Artificial Sequence
  Primer used for amplification of the GAPDH gene
tccaccaccctgttgctgta

SEQ ID NO: 34 - DNA, 25 bp, Artificial Sequence
  Primer used to show the expression of IGSF9 in human tumor cells
tcttatcttctctccgaccgggaag

SEQ ID NO: 35 - DNA, 25 bp, Artificial Sequence
  Primer used to show the expression of IGSF9 in human tumor cells
gccacagggctgatgtcttcaatgc

SEQ ID NO: 36 - DNA, 20 bp, Artificial Sequence
  Primer used for amplification of the GAPDH gene
accacagtccatgccatcac

SEQ ID NO: 37 - DNA, 20 bp, Artificial Sequence
  Primer used for amplification of the GAPDH gene
tccaccaccctgttgctgta

SEQ ID NO: 38 - DNA, 24 bp, Artificial Sequence
  Primer used to amplify IGSF9
gtgggccgggggctgcaaggccag

SEQ ID NO: 39 - DNA, 24 bp, Artificial Sequence
  Primer used to amplify IGSF9
agcagacaagacgatttcgctgaa

SEQ ID NO: 40 - DNA, 23 bp, Artificial Sequence
  Primer used to flank the region of IGSF9
caggaactggagcctgtgaccct

SEQ ID NO: 41 - DNA, 24 bp, Artificial Sequence
  Primer used to flank the region of IGSF9
ctctataaaagctgggggagcctt

SEQ ID NO: 42 - DNA, 23 bp, Artificial Sequence
  Primer used to amplify LIV-1
ggatggtgataatgggtgatggc

SEQ ID NO: 43 - DNA, 23 bp, Artificial Sequence
  Primer used to amplify LIV-1
ggtcactagcatcattgtgcagc

(19) United States
(12) Patent Application Publication
(10) Pub. No.: US 2004/0258616 A1
(43) Pub. Date: Dec. 23, 2004

(54) Compositions and methods for treating cancer using IGSF9 and LIV-1

(75) Inventors: Karen McLachlan, Encinitas, CA (US); Scott Glaser, San Diego, CA (US); Robert J. Peach, San Diego, CA (US); Tony Rowe, San Diego, CA (US)

Correspondence Address: STERNE, KESSLER, GOLDSTEIN & FOX PLLC, 1100 NEW YORK AVENUE, N.W., WASHINGTON, DC, 20005, US

(73) Assignee: IDEC Pharmaceuticals Corporation

(21) Appl. No.: 10/764,604

(22) Filed: Jan. 27, 2004

Related U.S. Application Data
(60) Provisional application No. 60/442,535, filed on Jan. 27, 2003.

(51) Int. Cl. A61K 51/00; A61K 39/395; C07K 16/46

(52) U.S. Cl. 424/1.49; 424/141.1; 530/388.15; 530/388.3

(57) ABSTRACT

Human IGSF9 and LIV-1 polypeptides and DNA (RNA) encoding such polypeptides are disclosed. The disclosed polypeptides and/or polynucleotide are particularly useful generating antibodies, both modified and native, which bind IGSF9 or LIV-1. Also disclosed are pharmaceutical compositions and vaccines comprising the antibodies, polypeptides and polynucleotides of the invention. Also disclosed are methods for utilizing such polypeptides for identifying ligands, antagonists and agonists to said polypeptides. Finally, methods comprising the above-mentioned compositions are disclosed for the treatment, diagnosis, and/or prognosis of neoplastic disorders.

This application claims the benefit of U.S. Provisional Application No. 60/442,535 filed Jan. 27, 2003, which is herein incorporated by reference.

### BACKGROUND OF THE INVENTION

1. Field of Invention

The present invention relates to compositions, specifically antibodies and antigen binding fragments, of IGSF9 and LIV-1, and methods of using said compositions for the detection and treatment of neoplastic disease.

2. Background Art

Cancer is the second leading cause of death in the United States, and accounts for over one-fifth of the total mortality. Cancer cells are defined by two heritable properties: they and their progeny (1) reproduce in defiance of the normal restraints and (2) invade and colonize territories normally reserved for other cells. The uncontrolled proliferation of cancer cells gives rise to a tumor, or neoplasm.

Expression of unique components of normal cellular products by cancer cells, is the fundamental hypothesis upon which tumor immunology is based. Substantial and convincing evidence now exists that clearly supports the concept that neoplastic transformation is associated with antigenic changes on mammalian cell surfaces (Reisfeld, R. A. and Cheresh, D. A., Ad Immunol 40:323-377 (1987). To define a large group of cell surface antigens that appear to have, at least, increased expression on human tumor cells, a variety of serologic strategies have been utilized (Old, L. J., Cancer Res 41:361-375 (1981); Rosenberg S A, (ed.) Serologic Analysis of Human Cancer Antigens. Academic Press, New York. 1980). Two such antigens are IGSF9 and LIV-1.

Members of the immunoglobulin protein superfamily, characterized by the presence of immunoglobulin-like domains, mediate both homophilic and heterophilic binding. (Doudney, et al., Genomics 79:663-670 (2002)). Immunoglobulin proteins often mediate signal transduction between an extracellular ligand and second-messenger cascades within the cell. As such, many immunoglobulin proteins have a transmembrane domain and a cytoplasmic carboxy-terminal sequence that interacts with the intracellular environment. For example, immunoglobulin proteins with cytoplasmic receptor tyrosine kinase or phosphatase domains exert their intracellular signaling influence directly through their enzymatic activity, while others act by associating with and activating intracellular kinases. Activation of tyrosine kinases of the src family by immunoglobulin ligand binding leads to effects on the dynamics of the cell cytoskeleton, providing an important link between cellular adhesion and cell shape changes associated with the morphogenetic movements of embryonic development.

IGSF9 (immunoglobulin superfamily member 9) is a novel member of the NCAM subclass of the immunoglobulin superfamily, which was identified during positional cloning efforts to isolate the mouse Lp gene. (Doudney, et al., Genomics 79:663-670 (2002)). A homolog of IGSF9 is the protein Turtle from Drosophila melanogaster, which is involved in neural development. In addition, IGSF9 may represent an important candidate for involvement in the formation and invasiveness of human tumors. Tumors with duplications of the chromosome 1q22-q23 region are frequently observed, and moreover, upregulation of the expression of immunoglobulin proteins is a common observation in human tumors, and may contribute to both the disregulation of cellular function and the invasiveness of neoplasia.

LIV-1 is an estrogen-regulated gene that is associated with metastatic breast cancer. Investigation of LIV-1 structure has revealed that it is a histidine-rich protein with a potential to bind and/or transport Zn²⁺ ions. Zn²⁺ is actively transported across biological membranes, and its uptake and efflux is tightly regulated because it is both essential and toxic to cells. (Taylor, K. M., IUBMB Life 49:249-253 (2000)).

LIV-1 is the only known hormone-regulated Zn²⁺-binding protein. Whether other Zn²⁺-binding proteins have a role in metastatic carcinomas remains to be determined. However, certain Zn²⁺-binding proteins in tissue arrays have been linked to cell death and neuronal disease.

### BRIEF SUMMARY OF THE INVENTION

The invention generally relates to, inter alia, compositions which can be used in the detection and treatment of cancer, and provides methods for cancer detection and treatment.

Experimental results provided below demonstrate that IGSF9 and LIV-1 are differentially expressed in various neoplastic cells. This differential expression allows for IGSF9 and LIV-1 to act as targets for the detection and treatment of a variety of neoplasms including breast, colon, ovary, lung and prostate cancer.

The present invention relates to an isolated antibody or antigen binding fragment thereof which associates with either IGSF9 or LIV-1 or a fragment of said proteins. More particularly, the isolated antibody or antigen binding fragment thereof may associate with IGSF9 between amino acids 21 to 718 as set forth in FIG. 1B (SEQ ID NO:2), between amino acids 21 to 734 of SEQ ID NO:8, the amino acids as set forth in SEQ ID NOS:22-27; or with LIV-1 between amino acids 28 to 317, 373 to 417, 674 to 678 or 742 to 749, as set forth in FIG. 22B (SEQ ID NO:29).

The invention is also directed to an isolated anti-IGSF9 or anti-LIV-1 antibody or antigen binding fragment, wherein said antibody or antigen binding fragment comprises a domain deleted antibody. The domain deleted antibody or antigen binding fragment thereof may further comprise a cytotoxic agent. In a preferred embodiment, the cytotoxic agent is a radionuclide.

The anti-IGSF9 or anti-LIV-1 antibody or antigen binding fragment of the invention may also be humanized or primatized.

The invention is also directed to an antibody or antigen fragment thereof which associates with IGSF9 or LIV-1, wherein said antibody or antigen binding fragment thereof inhibits one or more functions associated with IGSF9 or LIV-1.

The invention further relates to compositions comprising an antibody or antigen binding fragment thereof which associates with IGSF9 or LIV-1.

In a preferred embodiment, a method of treating a neoplastic disorder comprises a domain deleted anti-IGSF9 or anti-LIV-1 antibody or antigen binding fragment thereof covalently linked to one or more bifunctional chelators. The bifunctional chelator is selected from the group consisting of MX-DTPA and CHX-DTPA.

The invention is also directed to a method of treating a mammal exhibiting a neoplastic disorder comprising the step of administering a therapeutically effective amount of an antibody or antigen binding fragment thereof that associates with IGSF9 or LIV-1. Said method may further comprise administering a therapeutically effective amount of at least one chemotherapeutic agent to said mammal; wherein said chemotherapeutic agent and said antibody or antigen binding fragment thereof may be administered in any order or concurrently. In a preferred embodiment, anti-IGSF9 or anti-LIV-1 antibodies or antigen binding fragments are administered to a mammal in need of treatment. The anti-IGSF9 and anti-LIV-1 antibodies or antigen binding fragments may be modified to lack the C_{H}2 domain, and/or may be humanized, and further comprise a cytotoxic agent.

The present invention further relates to a vaccine for treating cancer comprising the IGSF9 or LIV-1 polypeptide or a fragment thereof and a physiologically acceptable carrier. In a preferred embodiment, the anti-cancer vaccine comprises amino acids 1 to 1163 or amino acids 21 to 718 of IGSF9 as set forth in FIG. 1B (SEQ ID NO:2); or amino acids 1 to 749, amino acids 28 to 317, or amino acids 373 to 417 of LIV-1 as set forth in FIG. 22B (SEQ ID NO:29). The vaccine may further comprise IGSF9 or LIV-1 peptides fused to a T helper peptide. In addition, the vaccine may further comprise a physiologically acceptable carrier such as an adjuvant or an immunostimulatory agent. In a more preferred embodiment, the vaccine further comprises the adjuvant PROVAX™. The present invention further relates to a method of using said vaccine to induce an immune response in a patient in need of treatment or prevention of cancer.

The present invention is also directed to a method of detecting overexpression of IGSF9 or LIV-1, or a fragment thereof, comprising:

a. obtaining a sample from an individual in need of diagnosis of cancer;

b. detecting expression of IGSF-9 or LIV-1, or a fragment thereof in said sample;

c. detecting expression of IGSF-9 or LIV-1, or a fragment thereof in a control sample from a normal individual, or normal tissue from the individual being diagnosed; and

d. comparing the level of expression of IGSF-9 or LIV-1 to that obtained in the control sample, wherein said comparison results in diagnosing cancer.

In one embodiment of the invention, overexpression is detected by nucleic acid amplification, hybridization or by using an antibody to IGSF9 or LIV-1, or an antigen binding fragment thereof. In another embodiment, the IGSF9 fragment comprises exons 5-10.

The present invention also relates to a method for determining the prognosis of an individual receiving a cancer treatment comprising:

a. obtaining a sample from said individual in need of prognosis of cancer treatment;

b. detecting expression of IGSF9 or LIV-1, or a fragment thereof in said sample;

c. detecting expression of IGSF9 or LIV-1, or a fragment thereof in a control sample from a normal individual, or normal tissue from the individual being diagnosed; and

d. comparing the level of expression of IGSF9 or LIV-1 to that obtained in the control sample, wherein said comparison results in a cancer prognosis.

In one embodiment, the IGSF9 fragment comprises exons 5-10.

The present invention also relates to a vaccine that comprises as an active ingredient, an anti-idiotypic antibody that immunologically mimics the IGSF9 or LIV-1 antigens or fragments thereof.

The present invention also relates to kits comprising the various polynucleotides, polypeptides, antibodies and antigen binding fragments described herein together with instructions for use thereof to treat or detect cancer.

The present invention also relates to a method of treating a neoplastic disorder in a mammal wherein neoplastic cells express the IGSF9 or LIV-1 antigens, comprising administering to said mammal a composition comprising a pharmaceutically effective amount of an antibody to IGSF9 or LIV-1, or an antigen binding fragment thereof. In a preferred embodiment, a vaccine comprising a pharmaceutically acceptable carrier and an anti-tumor immune-response-inducing effective amount of an immunogenic preparation comprising IGSF9 or LIV-1, is employed to induce anti-tumor immune response.

The present invention also relates to an antisense nucleic acid up to 50 nucleotides in length comprising at least an 8 nucleotide portion of IGSF9 or LIV-1 which inhibits the expression of IGSF9 or LIV-1. The antisense nucleic acids of the invention may comprise at least one modified internucleotide linkage. Further, the present invention relates to a method of inhibiting the expression of IGSF9 or LIV-1 in cells or tissues comprising contacting said cells or tissues with said antisense nucleic acids so that expression of IGSF9 or LIV-1 is inhibited.

The present invention is further related to isolated nucleic acid comprising the various forms of IGSF9 (SEQ ID NOS:1, 7, and 12-21). The present invention is also related to vectors and host cells which comprise SEQ ID NOS:1, 7, and 12-15. The present invention further relates to an isolated polypeptide and compositions comprising SEQ ID NOS:2, 8, and 22-27. The present invention also relates to a vaccine, as described above, comprising the polypeptides of SEQ ID NOS:2, 8, and 22-27 and a physiologically acceptable carrier.

The present invention is further related to an isolated nucleic acid comprising short form IGSF9-Ig (SEQ ID NO:3). The present invention is also related to vectors and host cells which comprise SEQ ID NO:3. The present invention is further related to an isolated polypeptide and a composition comprising the polypeptide of SEQ ID NO:4. The present invention further relates to a vaccine, as described above, for treating cancer comprising the polypeptide of SEQ ID NO:4 and a physiologically acceptable carrier.

The present invention is further related to an isolated nucleic acid comprising long form IGSF9-Ig (SEQ ID NO:5). The present invention is also related to vectors and host cells which comprise SEQ ID NO:5. The present invention is further related to a composition comprising the polypeptide of SEQ ID NO:6. The present invention further relates to a vaccine, as described above, for treating cancer comprising the polypeptide of SEQ ID NO:6 and a physiologically acceptable carrier.

### BRIEF DESCRIPTION OF THE FIGURES

FIGS. 1A and 1B are the nucleotide (SEQ ID NO:1) and protein (SEQ ID NO:2) sequences of human IGSF9, respectively. FIG. 1B shows the predicted signal sequence in bold, the predicted extracellular domain is underlined, and the predicted transmembrane domain is bolded and italicized.

FIG. 2 shows an electronic Northern profile showing the gene expression profile of IGSF9 as determined using the Gene Logic datasuite.

FIG. 3 shows IGSF9 expression in normal tissues. The upper panel shows IGSF9 expression, while the lower panel shows expression of Glyceraldehyde 3-phosphate dehydrogenase (GAPDH). The cDNA samples present in each lane are as follows: (1) brain, (2) placenta, (3) lung, (4) liver, (5) skeletal muscle, (6) kidney, (7) pancreas, (8) spleen, (9) thymus, (10) prostate, (11) testis, (12) ovary, (13) small intestine, (14) colon, (15) peripheral blood leukocytes, (16) positive control, and (17) negative control.

FIG. 4 shows IGSF9 expression in a panel of human ovarian tumor samples and cell lines. The upper panel shows IGSF9 expression, the lower panel shows GAPDH expression. The numbers above each lane correspond to ovarian tumor samples as follows: (1) moderately differentiated cystadenocarcinoma, (2) poorly differentiated papillary serous adenocarcinoma, (3) poorly differentiated papillary serous adenocarcinoma, (4) poorly differentiated endometriod adenocarcinoma, (5) papillary serous adenocarcinoma, (6) endometriod adenocarcinoma, (7) poorly differentiated adenocarcinoma, (8) poorly differentiated papillary serous adenocarcinoma, (9) Ovcar-3 cell line, (10) PA-1 cell line, (11) positive control, and (12) negative control.

FIG. 5 shows IGSF9 expression in breast tumor samples and matched normal breast samples. The upper gel shows IGSF9 expression, while the lower gel shows GAPDH expression. (N) normal tissue, (T) tumor tissue. The tumor samples are as follows: (Patient A) infiltrating ductal carcinoma, (patient B) infiltrating ductal carcinoma, (patient C) tubular adenocarcinoma, (patient D) infiltrating ductal carcinoma, (patient E) infiltrating ductal carcinoma, (patient T) high grade in situ & invasive ductal carcinoma, (patient X) ductal adenocarcinoma, (patient W) mixed ductal and lobular adenocarcinoma, (patient GH19) high grade invasive ductal carcinoma, (patient GH17) low grade intraductal carcinoma.

FIG. 6 shows IGSF9 expression in lung tumors. The upper panels shows IGSF9 expression, while the lower panel shows GAPDH expression. (N) normal sample, (T) tumor sample. The tumor samples analyzed were as follows: (Patient A) infiltrating ductal carcinoma, (patient B) squamous cell keratinizing carcinoma, (patient C) adenosquamous carcinoma, (patient D) keratinizing squamous cell carcinoma, (patient E) squamous cell carcinoma.

FIG. 7 shows IGSF9 expression in colon tumors. The upper panel shows IGSF9 expression, while the lower panel shows GAPDH expression. Samples are as follows: (1) grade 3 adenocarcinoma, (2) grade 2 adenocarcinoma, (3) grade 1 adenocarcinoma, (4) grade 2 adenocarcinoma, (5) colorectal cancer cell line HCT116.

FIG. 8 shows IGSF9 expression in human tumor cell lines by RT-PCR analysis. Relative IGSF9 expression was determined in pancreatic (speckled), ovarian (vertical lines), breast (diagonal down lines), lung (filled speckled), and colon (diagonal up lines) cell lines.

FIG. 9 shows the nucleotide and amino acid sequence of various IGSF9 constructs. FIGS. 9A and 9B show the nucleotide (SEQ ID NO:3) and amino acid (SEQ ID NO:4) sequence of short form soluble IGSF9-Ig, respectively. FIGS. 9C and 9D show the nucleotide (SEQ ID NO:5) and amino acid (SEQ ID NO:6) sequence of long form soluble IGSF9-Ig, respectively. FIGS. 9E and 9F show the nucleotide (SEQ ID NO:7) and amino acid (SEQ ID NO:8) sequence of long form full length IGSF9, respectively. FIG. 9G is a protein sequence comparison of long and short form IGSF9 (SEQ ID NOS:9-11). FIG. 9H is the nucleotide sequence of alternate splice forms of IGSF9 in the region of exons 5-11 from tumor xenograft samples (SEQ ID NOS:12-15).

FIG. 10 shows an SDS-PAGE analysis of recombinantly expressed and purified IGSF9 polypeptides. Lanes 1 and 2 depict the short and long form of soluble IGSF9-Ig, respectively.

FIG. 11 shows a Northern blot analysis of IGSF9 in stably transfected CHO cell lines. The samples in each lane are as follows: (1) untransfected wild-type CHO DG44 cells; (2) stable CHO 5 nM methotrexate (MTX) amplificant expressing full length short form IGSF9; (3) stable CHO 5 nM MTX amplificant expressing short form soluble IGSF9-Ig; (4) stable CHO 50 nM MTX amplificant expressing short form soluble IGSF9-Ig; (5) stable CHO G418 clone expressing long form soluble IGSF9-Ig.

FIG. 12 shows anti-IGSF9 antibody titers from mouse sera determined by ELISA against purified short form IGSF9-Ig.

FIG. 13 shows a FACS analysis of short form IGSF9 surface expression on transfected G418-resistant and MTX-amplified CHO DG44 cell lines stably expressing short form IGSF9. IGSF9 surface expression is shown in untransfected CHO DG44 cells (DG44); G418 resistant cells (G418); 5 nM MTX amplificant (5 nM); and 50 nM MTX amplificant (50 nM).

FIG. 14 shows a FACS analysis of long form IGSF9 surface expression on transfected G418-resistant and MTX-amplified CHO DG44 cell lines stably expressing the long form of IGSF9. IGSF9 surface expression is shown in untransfected CHO DG44 cells (CHO); and G418-resistant cells (G418).

FIG. 15 shows a FACS analysis of endogenous IGSF9 surface expression in NCI-H69 tumor cells. 2o control cells, an isotype matched control antibody (2B8), and multiple concentrations of the primary detecting antibody 8F3 were tested.

FIG. 16 shows a western blot analysis of IGSF9 expression in human tumor cell lines. Two different exposure times are shown: 30 minutes (left panel) and 5 seconds (right panel, showing lanes 2 and 3 only). The cell line used in each lane is as follows: (1) mock-transfected COS-7 cells (5 μg); (2) COS-7 cells transiently transfected with full-length IGSF9 (5 μg); (3); stable CHO G418 clone expressing full-length IGSF9 (50 μg); (4) MDA-MB-468 breast cancer cell line (50 μg); (5) ZR-75-1 breast cancer cell line (50 μg); (6) NCI-H69 small cell lung cancer cell line (50 μg); (7) Ovcar-3 ovarian cancer cell line (50 μg); (8) PA-1 ovarian cancer cell line (50 μg).

FIG. 17 shows cell surface IGSF9 expression in the breast tumor cell line ZR-75 as visualized by immunofluorescence microscopy.

FIG. 18 shows a FACS analysis of cell surface IGSF9 expression in Ovcar-3 and NCI-H69 murine tumor xenografts and cultured cells.

FIG. 19 shows an RT-PCR analysis of IGSF9 expression in two in vivo passages (P0 and P1) of LS174T and NCI-H69 tumor cell lines, and Ovcar-3 cells derived from murine xenografts.

FIG. 20 shows that alternate splice forms of IGSF9 are expressed by murine xenograft tumors. FIG. 20A shows PCR products obtained from: (1) NCI-H69 tumor cell line; (2) Ovcar-3 tumor cell line; (3) NCI-H69 mouse xenograft; (4) Ovcar-3 mouse xenograft; and (5) negative control. FIG. 20B shows a schematic representation showing the alignment of novel splice variants found in Ovcar-3 and NCI-H69 tumor xenografts. The upper diagram shows exons 5-10 of known IGSF9 variants (short and long form). The lower diagram shows exons 5-11 of novel IGSF9 isoforms.

FIG. 21 shows IGSF9 sequence alignments of novel IGSF9 isoforms derived from murine xenograft tissue. FIG. 21A shows an alignment of the partial long form nucleotide sequence of nucleotides 1138-1155 of the open reading frame containing exons 8-10 aligned with the corresponding partial sequence from the unique splice variants expressed in Ovcar-3 and NCI-H69 xenograft tumors. FIG. 21B shows an alignment of the translated amino acid sequence of amino acids 285-426 contained in exons 8-11 aligned with the corresponding partial sequence from the unique splice variants expressed in Ovcar-3 and NCI-H69 xenograft tumors. The sequences represented in the alignment are as follows: (1) long form IGSF9 (SEQ ID NOS:16 and 22); (2) sequence obtained from Ovcar-3 xenograft, clone 2 (SEQ ID NOS:17 and 23); (3) sequence obtained from Ovcar-3 xenograft, clone 1 (SEQ ID NOS:18 and 24); (4) sequence obtained from NCI-H69 xenograft clone 1 (SEQ ID NOS:19 and 25); (5) sequence obtained from NCI-H69 xenograft clone 2 (SEQ ID NOS:20 and 26); and (6) consensus sequence (SEQ ID NOS:21 and 27).

FIGS. 22A and 22B are the nucleotide (SEQ ID NO:28) and protein (SEQ ID NO:29) sequences of human LIV-1, respectively. FIG. 22B shows the predicted signal sequence in bold, the predicted extracellular domains are underlined, and the predicted transmembrane domains are bolded and italicized.

FIG. 23 shows an electronic Northern profile showing the gene expression profile of LIV-1 using the Gene Logic datasuite.

FIG. 24 shows LIV-1 expression in normal tissues. The upper panel shows LIV-1 expression, while the lower panel shows GAPDH expression. The cDNA samples present in each lane are as follows: (1) heart, (2) brain, (3) placenta, (4) lung, (5) liver, (6) skeletal muscle, (7) kidney, (8) pancreas, (9) negative control, and (10) positive control.

FIG. 25 shows LIV-1 expression in breast tumor samples and matched normal breast samples. The upper gels show LIV-1 expression, while the lower gels show GAPDH expression. The arrowhead on the right of the figure denotes the anticipated size of the LIV-1 PCR product. The tumor samples are as follows: (1-patient A) infiltrating ductal carcinoma, (2-patient B) infiltrating ductal carcinoma, (3-patient C) tubular adenocarcinoma, (4-patient D) infiltrating ductal carcinoma, (5-patient E) infiltrating ductal carcinoma, (6—patient A) normal, (7-patient B) normal, (8-patient C) normal, (9-patient D) normal, (10-patient E) normal, (11) negative control, (12) positive control, (13-patient G19) high grade invasive ductal carcinoma, (14-patient G17) low grade intraductal carcinoma, (15-patient X) ductal adenocarcinoma, (16—patient W) mixed ductal and lobular adenocarcinoma, (17-patient T) high grade in situ & invasive ductal carcinoma, (18-patient G19) normal, (19-patient G17) normal, (20-patient X) normal, (21-patient W) normal, (22-patient T) normal, (23) negative control, and (24) positive control.

FIG. 26 shows LIV-1 expression in colon tumors. The upper panel shows LIV-1 expression, while the lower panel shows GAPDH expression. Samples are as follows: (1) grade 3 adenocarcinoma, (2) grade 2 adenocarcinoma, (3) grade 1 adenocarcinoma, (4) grade 2 adenocarcinoma, (5) colorectal cancer cell line HCT 116, (6) positive control, and (7) negative control.

### DETAILED DESCRIPTION OF THE INVENTION

It is a discovery of the present invention that the IGSF9 and LIV-1 gene are differentially expressed between neoplastic cells, especially neoplasms of the breast, ovary, colon, lung, and prostate, and normal cells. Overexpression of these genes can be used as a marker for cancer. This information can be utilized to make diagnostic and therapeutic reagents specific for both the genes and their expression products, specifically antibodies and antigen binding fragments thereof. It can also be used in diagnostic and therapeutic methods that will aid in providing the appropriate treatment regimens for cancer patients, especially those having breast, ovary, colon, lung, or prostate cancer.

Antibodies of the Present Invention

Peptides from IGSF9 or LIV-1 can be used to raise polyclonal and monoclonal antibodies. The present invention is predicated, at least in part, on the fact that antibodies or antigen binding fragments which are immunoreactive with antigens associated with neoplastic cells may be modified or altered to provide enhanced biochemical characteristics and improved efficacy when used in therapeutic protocols on cancer patients. Preferably, the modified antibodies will be associated with a cytotoxic agent such as a radionuclide or antineoplastic agent.

The term “antibody” as used herein refers to immunoglobulin molecules and immunologically active portions of immunoglobulin (Ig) molecules. Such antibodies include, but are not limited to, polyclonal, monoclonal, chimeric, single chain, Fab, Fab′ and Fab′₂ fragments, Fv, and an Fab expression library. In general, an antibody molecule obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of antibody species.

It has been shown that fragments of an antibody can perform the function of binding antigens. As used herein “antigen binding fragments” include, but are not limited to: (i) the Fab fragment consisting of V_{L}, V_{H}, C_{L} and C_{H}1 domains; (ii) the Fd fragment consisting of the V_{H} and C_{H}1 domains; (iii) the Fv fragment consisting of the V_{L} and V_{H} domains of a single antibody; (iv) the dAb fragment (Ward, E. S. et al., Nature 341:544-546 (1989)) which consists of a V_{H} domain; (v) isolated CDR regions; (vi) F(ab′)₂ fragments (vii) single chain Fv molecules (scFv), wherein a V_{H} domain and a V_{L} domain are linked by a peptide linker which allows the two domains to associate to form an antigen binding site (Bird, et al., Science 242:423-426 (1988); Huston et al., Proc. Natl. Acad. Sci. USA 85:5879-5883 (1988)); (viii) bispecific single chain Fv dimers (PCT/US92/09965) and (ix) diabodies, multivalent or multispecific fragments constructed by gene fusion (WO94/13804; P. Holliger et al., Proc. Natl. Acad. Sci. USA 90:6444-6448 (1993).

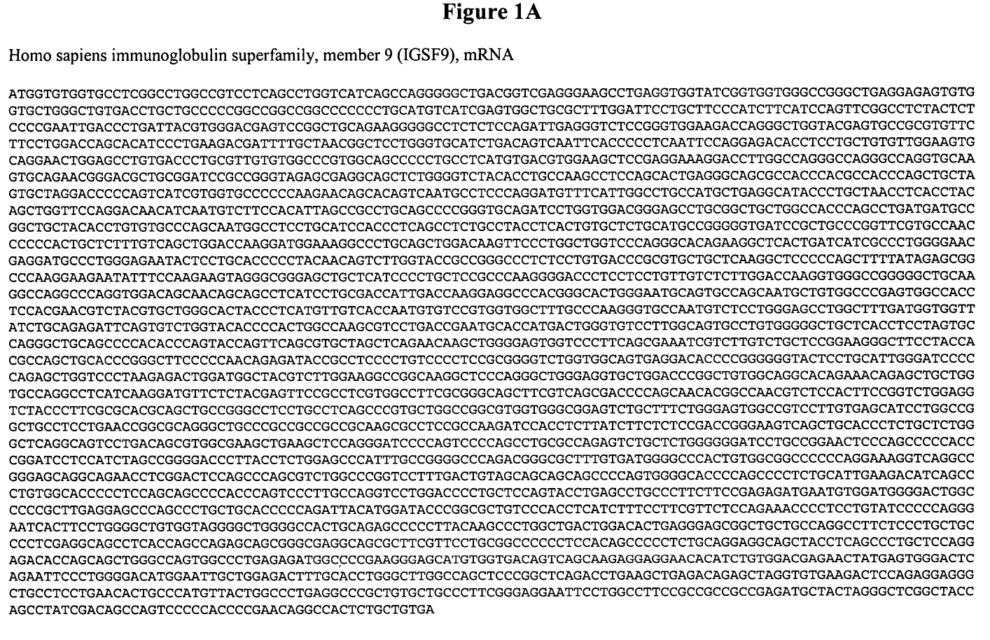

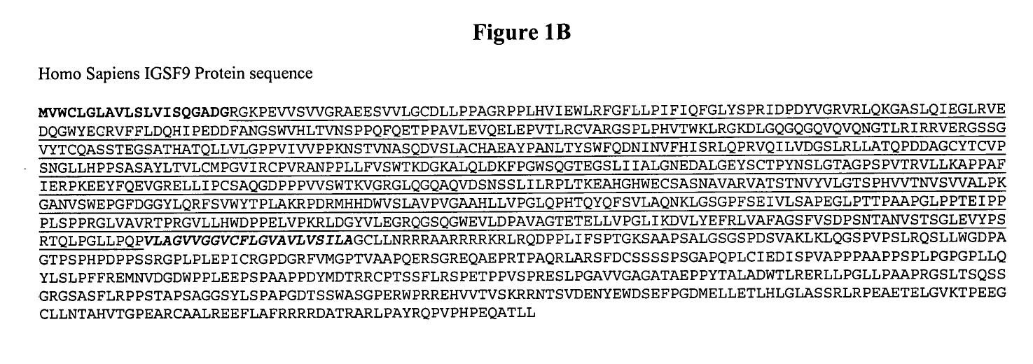

An isolated polypeptide of the invention may be intended to serve as an antigen, or a portion or fragment thereof, and additionally can be used to generate antibodies that immunospecifically bind the antigen, using standard techniques for polyclonal and monoclonal antibody preparation. The full-length protein can be used or, alternatively, the invention provides antigenic peptide fragments of IGSF9 and LIV-1 for use as immunogens. An antigenic peptide fragment comprises at least 6 amino acid residues of the amino acid sequence of the full-length IGSF9 of LIV-1 proteins, such as the amino acid sequences shown in FIGS. 1B, 9B, 9D, 9F, 21B, or 22B (SEQ ID NOS:2, 4, 6, 8, 22-27, or 29), and encompasses an epitope thereof such that an antibody raised against the peptide forms a specific immune complex with the full-length protein or with any fragment that contains the epitope. Preferably, the antigenic peptide comprises at least 10 amino acid residues, or at least 15 amino acid residues, or at least 20 amino acid residues, or at least 30 amino acid residues.

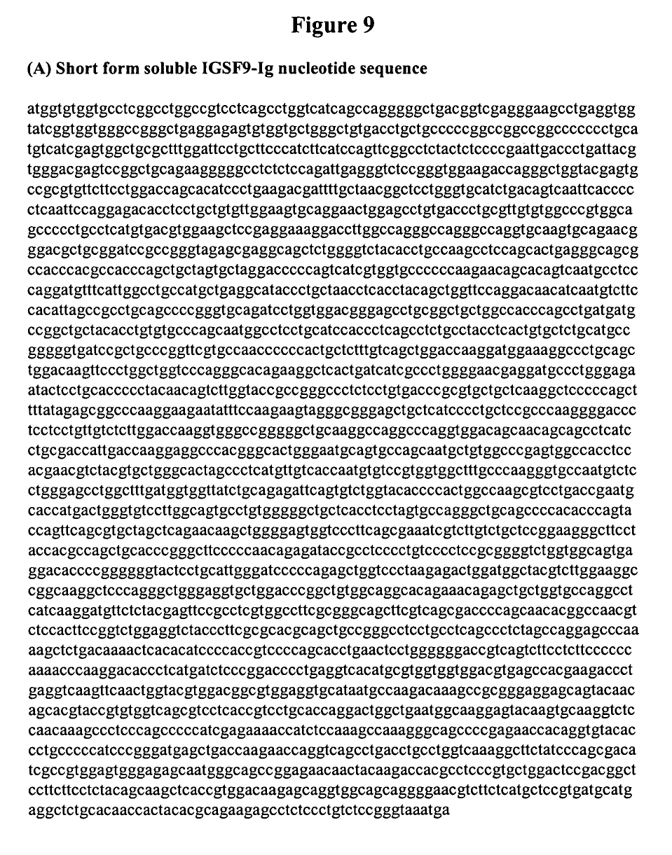

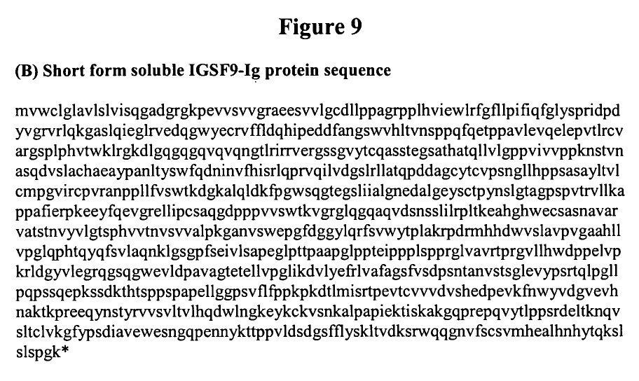

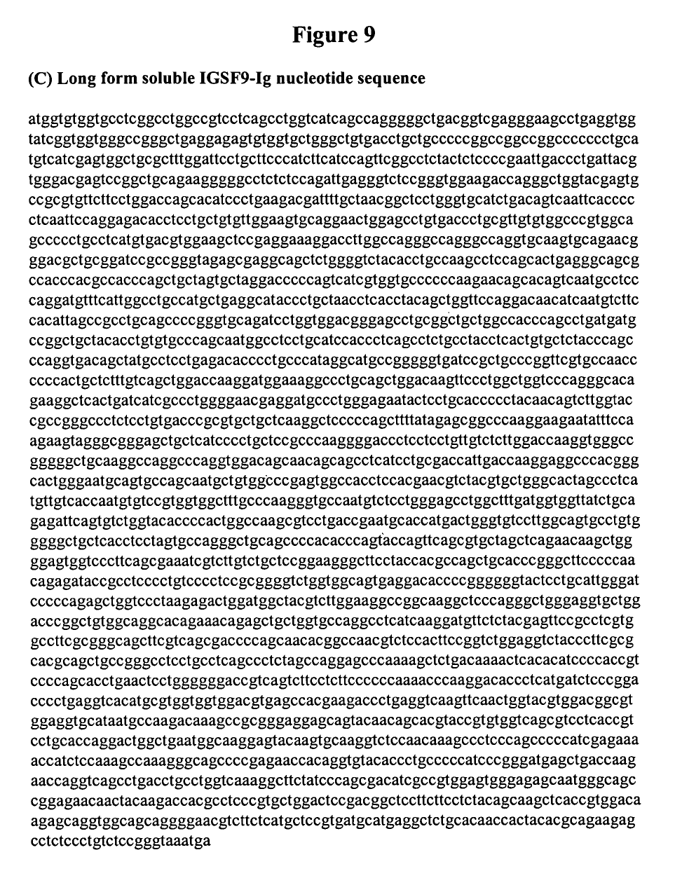

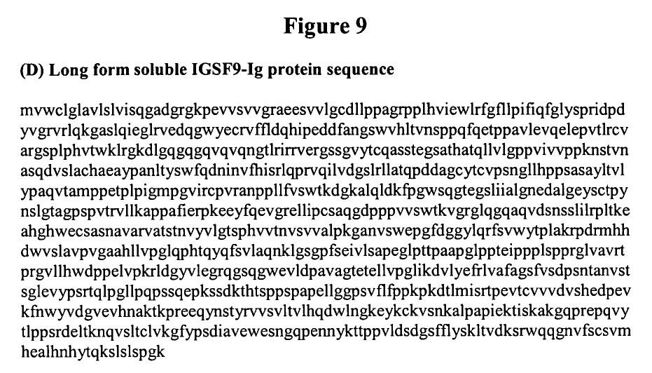

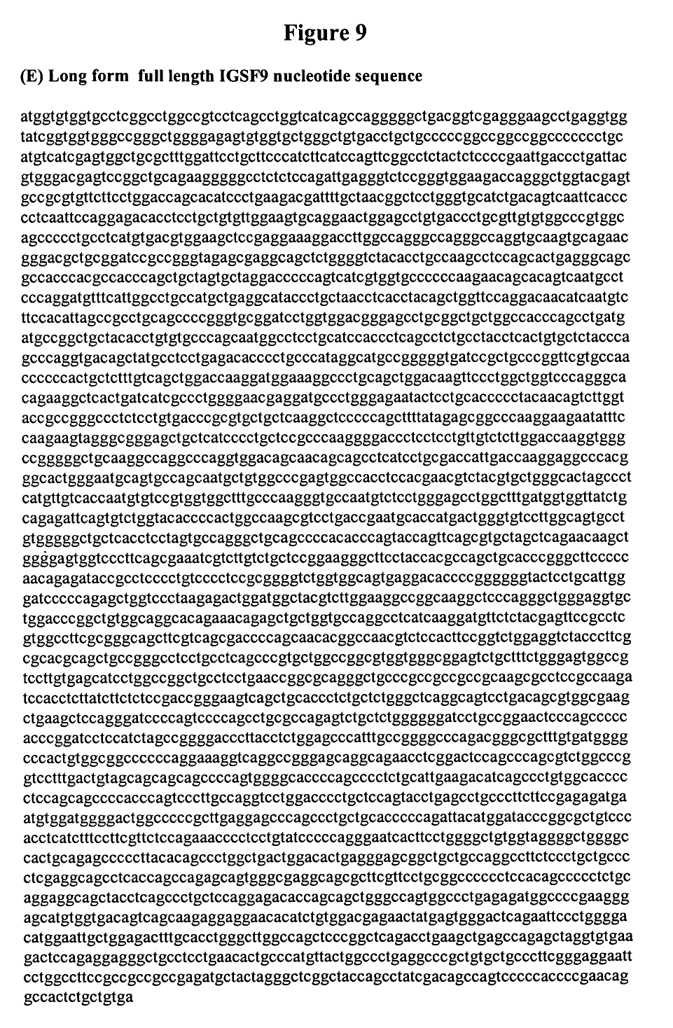

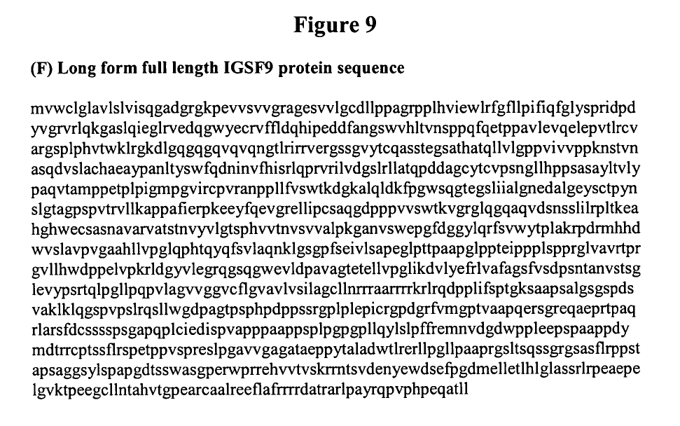

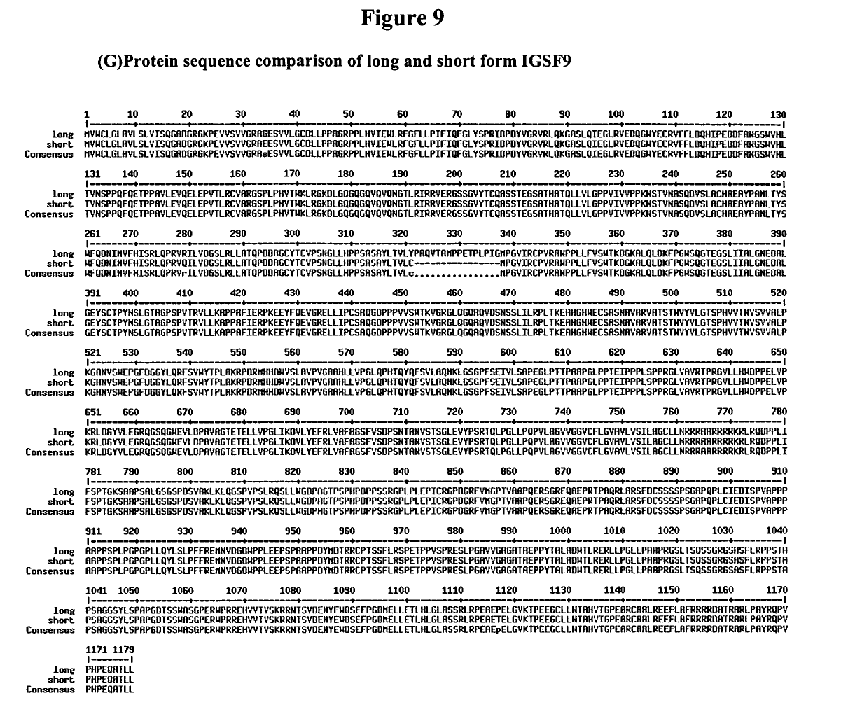

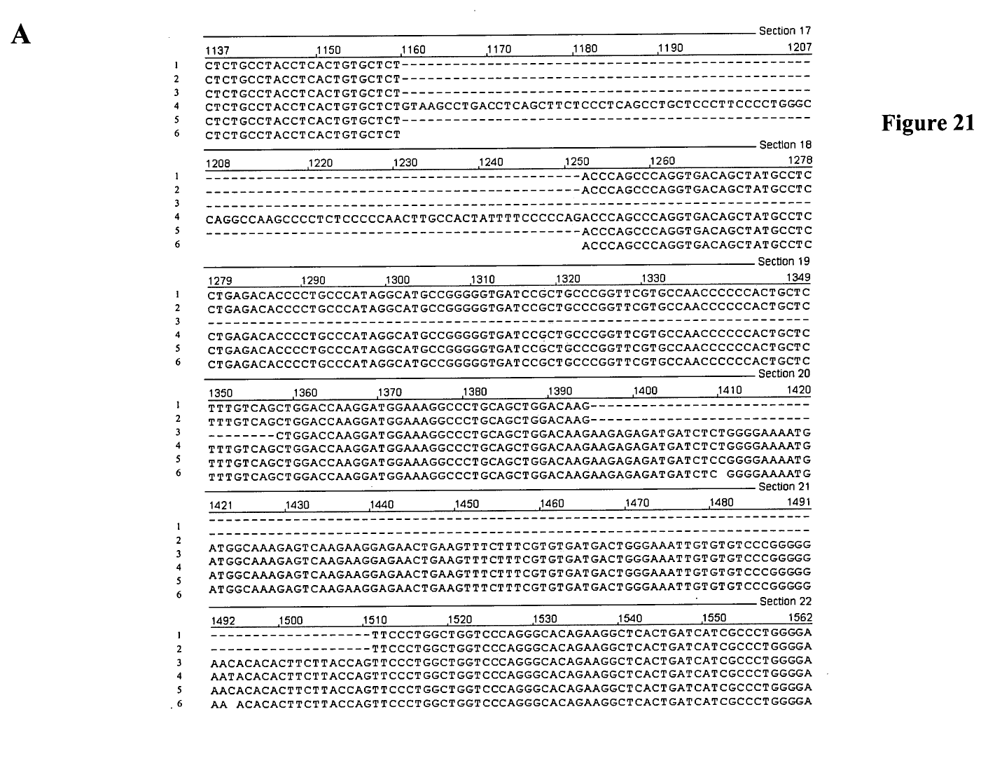

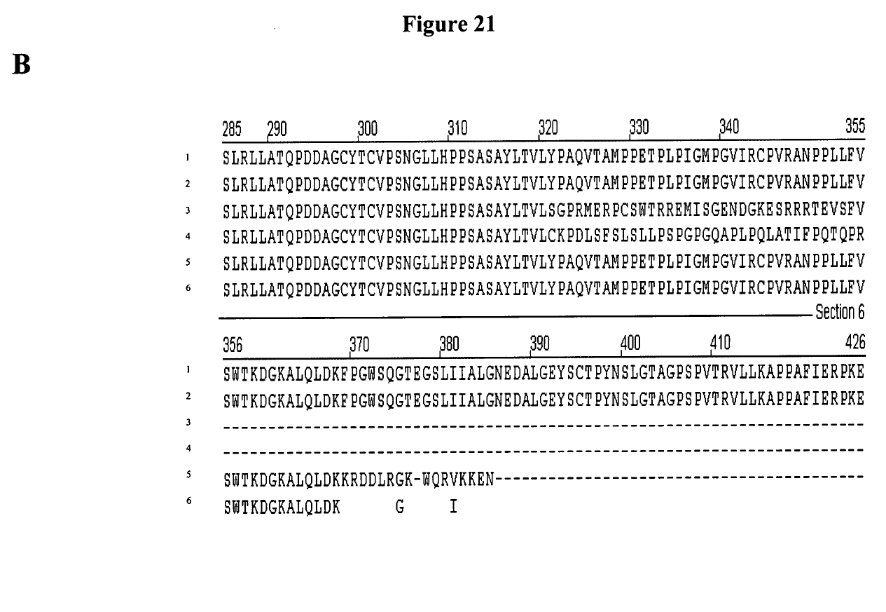

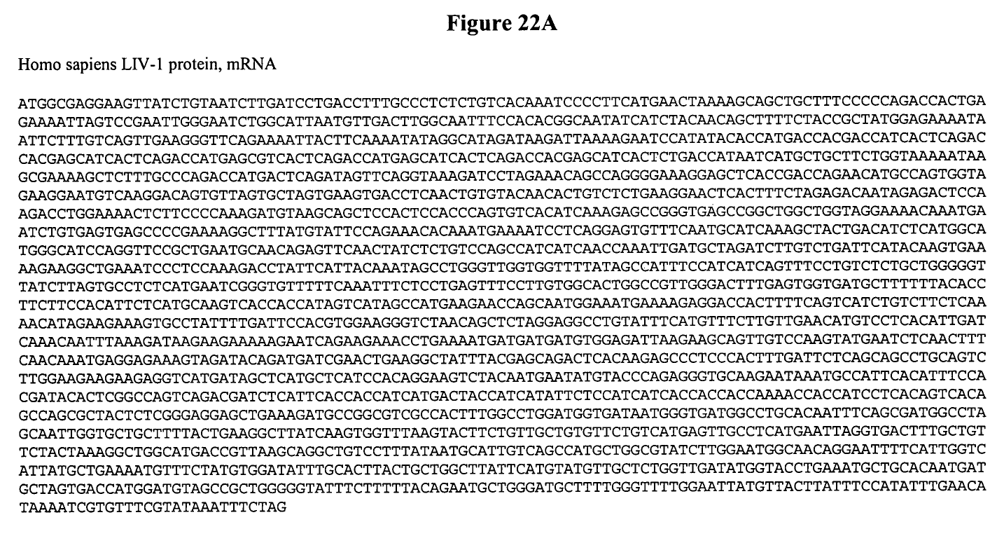

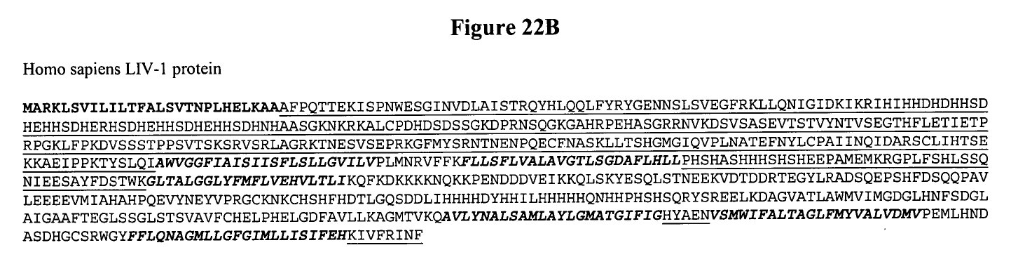

In certain embodiments of the invention, at least one epitope encompassed by the antigenic peptide is a region of IGSF9 or LIV-1 that is located on the surface of the protein, e.g., a hydrophilic region. A hydrophobicity analysis of the human IGSF9 and LIV-1 protein sequences (FIGS. 1B and 22B) has indicated which regions of these proteins are particularly hydrophilic and, therefore, are likely to encode surface residues useful for targeting antibody production (Kyte and Doolittle, J. Mol. Biol. 157:105-142 (1982)). Therefore, preferred epitopes encompassed by the antigenic peptides are regions of IGSF9 and LIV-1 that are located on its surface, for example, from about amino acid 21 to about amino acid 718 of IGSF9 (FIG. 1B); from about amino acid 21 to about amino acid 734 of IGSF9 (FIG. 9F); the amino acid sequences as shown in FIG. 21B; or from about amino acid 28 to about amino acid 317, from about amino acid 373 to about amino acid 417, from about amino acid 674 to about amino acid 678, or from about amino acid 742 to about amino acid 749 of LIV-1 (FIG. 22B) (SEQ ID NOS:2, 4, 6, 8, 22-27, or 29).

For the production of polyclonal antibodies, various suitable host animals (e.g., rabbit, goat, mouse or other mammal) may be immunized by one or more injections with IGSF9 or LIV-1 peptides, synthetic variants, derivatives, or fragments thereof. An appropriate immunogenic preparation can contain, for example, the naturally occurring immunogenic protein, a chemically synthesized polypeptide representing the immunogenic protein, or a recombinantly expressed polypeptide of the immunogenic protein, or fragment thereof. Furthermore, the protein may be conjugated to a second protein known to be immunogenic in the mammal being immunized. Examples of such immunogenic proteins include but are not limited to keyhole limpet hemocyanin, serum albumin, bovine thyroglobulin and soybean trypsin inhibitor. The preparation can further include an adjuvant. Various adjuvants used to increase the immunological response include, but are not limited to, Freund's (complete and incomplete), mineral gels (e.g., aluminum hydroxide), MPL-TDM (monophosphoryl Lipid A-synthetic trehalose dicorynomycolate), and PROVAX™.

Polyclonal antibodies directed against IGSF9 or LIV-1 can be isolated from the immunized mammal and further purified using techniques well known in the art such as affinity chromatography using protein A or protein G.

While the resulting antibodies may be harvested from the serum of the mammal to provide polyclonal preparations, it is often desirable to isolate individual lymphocytes from the spleen, lymph nodes or peripheral blood, to provide homogenous preparations of monoclonal antibodies. Preferably, the lymphocytes are obtained from the spleen.

“Monoclonal antibodies” (MAbs) as used herein, refers to a population of antibody molecules that contain only one molecular species of antibody molecule consisting of a unique light chain gene product and a unique heavy chain gene product. In particular, the complementarity determining regions of the MAb are identical in all the molecules of the population. MAbs, thus contain an antigen binding site capable of immunoreacting with a particular epitope of the antigen characterized by a unique binding affinity for it.

In this well known process (Kohler et al., Nature 256:495 (1975)) the relatively short-lived, or mortal, lymphocytes from a mammal which have been injected with antigen are fused with an immortal tumor cell line (e.g. a myeloma cell line), thus producing hybrid cells or “hybridomas” which are both immortal and capable of producing the genetically coded antibody of the B cell. The resulting hybrids are segregated into single genetic strains by selection, dilution, and regrowth with each individual strain comprising specific genes for the formation of a single antibody.

Hybridoma cells thus prepared are seeded and grown in a suitable culture medium that preferably contains one or more substances that inhibit the growth or survival of the unfused, parental myeloma cells. Those skilled in the art will appreciate that reagents, cell lines and media for the formation, selection and growth of hybridomas are commercially available from a number of sources and standardized protocols are well established. Generally, culture medium in which the hybridoma cells are growing is assayed for production of MAbs against the desired antigen. Preferably, the binding specificity of the monoclonal antibodies produced by hybridoma cells is determined by immunoprecipitation or by an in vitro assay, such as a radioimmunoassay (RIA) or enzyme-linked immunosorbent assay (ELISA). After hybridoma cells are identified that produce antibodies of the desired specificity, affinity and/or activity, the clones may be subcloned by limiting dilution procedures and grown by standard methods (Goding, Monoclonal Antibodies: Principles and Practice, pp 59-103 (Academic Press, 1986)). It will further be appreciated that the monoclonal antibodies secreted by the subclones may be separated from culture medium, ascites fluid or serum by conventional purification procedures such as, for example, protein-A, hydroxylapatite chromatography, gel electrophoresis, dialysis or affinity chromatography.

As used herein the term “modified antibody” shall be held to mean any antibody, or antigen binding fragment or recombinant thereof, immunoreactive with either IGSF9 or LIV-1 in which at least a fraction of one or more of the constant region domains has been deleted or otherwise altered so as to provide desired biochemical characteristics such as increased tumor localization or reduced serum half-life when compared with a whole, unaltered antibody of approximately the same binding specificity. In preferred embodiments, the modified antibodies of the present invention have at least a portion of one of the constant domains deleted. For the purposes of this disclosure, such constructs shall be termed “domain deleted.” Preferably, one entire domain of the constant region of the modified antibody will be deleted and even more preferably the entire C_{H}2 domain will be deleted. As will be discussed in more detail below, each of the desired variants may readily be fabricated or constructed from a whole precursor or parent antibody using well known techniques.

Those skilled in the art will appreciate that the compounds, compositions and methods of the present invention are useful for treating any neoplastic disorder, tumor or malignancy that exhibits a polypeptide of the present invention. As discussed above, the modified antibodies of the present invention are immunoreactive with either IGSF9 or LIV-1. That is, the antigen binding portion (i.e. the variable region or immunoreactive fragment or recombinant thereof) of the disclosed modified antibodies binds to either IGSF9 or LIV-1 at the site of the malignancy. More generally, modified antibodies useful in the present invention may be obtained or derived from any antibody (including those previously reported in the literature) that reacts with IGSF9 or LIV-1. Further, the parent or precursor antibody, or fragment thereof, used to generate the disclosed modified antibodies may be murine, human, chimeric, humanized, non-human primate or primatized. In other preferred embodiments the modified antibodies of the present invention may comprise single chain antibody constructs (such as that disclosed in U.S. Pat. No. 5,892,019 which is incorporated herein by reference) having altered constant domains as described herein. Consequently, any of these types of antibodies modified in accordance with the teachings herein are compatible with this invention.

The modified antibodies of the present invention preferably associate with, and bind to, IGSF9 or LIV-1. Accordingly, as will be discussed in some detail below, the modified antibodies of the present invention may be derived, generated or fabricated from any one of a number of antibodies that react with IGSF9 or LIV-1. In preferred embodiments, the modified antibodies will be derived using common genetic engineering techniques whereby at least a portion of one or more constant region domains are deleted or altered so as to provide the desired biochemical characteristics such as reduced serum half-life. More particularly, as will be exemplified below, one skilled in the art may readily isolate the genetic sequence corresponding to the variable and/or constant regions of the subject antibody and delete or alter the appropriate nucleotides to provide the modified antibodies of this invention. It will further be appreciated that the modified antibodies may be expressed and produced on a clinical or commercial scale using well-established protocols.

In selected embodiments, modified antibodies useful in the present invention will be derived from known antibodies to IGSF9 or LIV-1. This may readily be accomplished by obtaining either the nucleotide or amino acid sequence of the parent antibody and engineering the modifications as discussed herein. For other embodiments it may be desirable to only use the antigen binding region (e.g., variable region or complementary determining regions) of the known antibody and combine them with a modified constant region to produce the desired modified antibodies. Compatible single chain constructs may be generated in a similar manner. In any event, it will be appreciated that the antibodies of the present invention may also be engineered to improve affinity or reduce immunogenicity as is common in the art. For example, the modified antibodies of the present invention may be derived or fabricated from antibodies that have been humanized or chimerized. Thus, modified antibodies consistent with present invention may be derived from and/or comprise naturally occurring murine, primate (including human) or other mammalian monoclonal antibodies, chimeric antibodies, humanized antibodies, primatized antibodies, bispecific antibodies or single chain antibody constructs as well as immunoreactive fragments of each type.

In addition to the antibodies discussed above, it may be desirable to provide modified antibodies derived from or comprising antigen binding regions of novel antibodies generated using immunization coupled with common immunological techniques discussed above.

In other compatible embodiments, DNA encoding the desired monoclonal antibodies may be readily isolated and sequenced using conventional procedures (e.g., by using oligonucleotide probes that are capable of binding specifically to genes encoding the heavy and light chains of murine antibodies). The isolated and subcloned hybridoma cells serve as a preferred source of such DNA. Once isolated, the DNA may be placed into expression vectors, which are then transfected into prokaryotic or eukaryotic host cells such as E. coli cells, simian COS cells, Chinese Hamster Ovary (CHO) cells or myeloma cells that do not otherwise produce immunoglobulins. More particularly, the isolated DNA (which may be modified as described herein) may be used to clone constant and variable region sequences for the manufacture antibodies as described in Newman et al., U.S. Pat. No. 5,658,570 which is incorporated by reference herein. Essentially, this entails extraction of RNA from the selected cells, conversion to cDNA, and amplification thereof by PCR using immunoglobulin specific primers. As will be discussed in more detail below, transformed cells expressing the desired antibody may be grown up in relatively large quantities to provide clinical and commercial supplies of the immunoglobulin.

Those skilled in the art will also appreciate that DNA encoding antibodies or antibody fragments may also be derived from antibody phage libraries as set forth, for example, in EP 368 684 B1 and U.S. Pat. No. 5,969,108 each of which is incorporated herein by reference. Several publications (e.g., Marks et al. Bio/Technology 10:779-783 (1992)) have described the production of high affinity human antibodies by chain shuffling, as well as combinatorial infection and in vivo recombination as a strategy for constructing large phage libraries. Such procedures provide viable alternatives to traditional hybridoma techniques for the isolation and subsequent cloning of monoclonal antibodies and, as such, are clearly within the purview of this invention.

Yet other embodiments of the present invention comprise the generation of substantially human antibodies in transgenic animals (e.g., mice) that are incapable of endogenous immunoglobulin production (see e.g., U.S. Pat. Nos. 6,075,181, 5,939,598, 5,591,669 and 5,589,369 each of which is incorporated herein by reference). For example, it has been described that the homozygous deletion of the antibody heavy-chain joining region in chimeric and germ-line mutant mice results in complete inhibition of endogenous antibody production. Transfer of a human immunoglobulin gene array in such germ line mutant mice will result in the production of human antibodies upon antigen challenge. Another preferred means of generating human antibodies using SCID mice is disclosed in commonly-owned, U.S. Pat. No. 5,811,524 which is incorporated herein by reference. It will be appreciated that the genetic material associated with these human antibodies may also be isolated and manipulated as described herein.

Yet another highly efficient means for generating recombinant antibodies is disclosed by Newman, Biotechnology 10: 1455-1460 (1992). Specifically, this technique results in the generation of primatized antibodies that contain monkey variable domains and human constant sequences. This reference is incorporated by reference in its entirety herein. Moreover, this technique is also described in commonly assigned U.S. Pat. Nos. 5,658,570, 5,693,780 and 5,756,096 each of which is incorporated herein by reference.

It will further be appreciated that the scope of this invention encompasses all alleles, variants and mutations of the DNA sequences described herein.

As is well known, RNA may be isolated from the original hybridoma cells or from other transformed cells by standard techniques, such as guanidinium isothiocyanate extraction and precipitation followed by centrifugation or chromatography. Where desirable, mRNA may be isolated from total RNA by standard techniques such as chromatography on oligodT cellulose. Techniques suitable to these purposes are familiar in the art and are described in the foregoing references.

cDNAs that encode the light and the heavy chains of the antibody may be made, either simultaneously or separately, using reverse transcriptase and DNA polymerase in accordance with well known methods. It may be initiated by consensus constant region primers or by more specific primers based on the published heavy and light chain DNA and amino acid sequences. As discussed above, PCR also may be used to isolate DNA clones encoding the antibody light and heavy chains. In this case the libraries may be screened by consensus primers or larger homologous probes, such as mouse constant region probes.

DNA, typically plasmid DNA, may be isolated from the cells as described herein, restriction mapped and sequenced in accordance with standard, well known techniques set forth in detail in the foregoing references relating to recombinant DNA techniques. Of course, the DNA may be modified according to the present invention at any point during the isolation process or subsequent analysis.

According to the present invention, techniques can be adapted for the production of single-chain antibodies specific to a polypeptide of the invention (see U.S. Pat. No. 4,946,778). In addition, methods can be adapted for the construction of Fab expression libraries (Huse, et al., Science 246:1275-1281 (1989)) to allow rapid and effective identification of monoclonal Fab fragments with the desired specificity for IGSF9 or LIV1, or derivatives, fragments, analogs or homologs thereof. Antibody fragments that contain the idiotypes to a polypeptide of the invention may be produced by techniques in the art including, but not limited to: (a) an F(ab′)₂ fragment produced by pepsin digestion of an antibody molecule; (b) an Fab fragment generated by reducing the disulfide bridges of an F(ab′)₂ fragment, (c) an Fab fragment generated by the treatment of the antibody molecule with papain and a reducing agent, and (d) Fv fragments.

Bispecific antibodies are also within the scope of the invention. Bispecific antibodies are monoclonal, preferably human or humanized, antibodies that have binding specificities for at least two different antigens. In the present case, one of the binding specificities is for an antigenic polypeptide of the invention (IGSF9 or LIV-1, or a fragment thereof), while the second binding target is any other antigen, and advantageously is a cell surface protein, or receptor or receptor subunit.

Methods for making bispecific antibodies are known in the art. Traditionally the recombinant production of bispecific antibodies is based on the co-expression of two immunoglobulin heavy chain/light chain pairs, where the two heavy chains have different specificities (Milstein and Cuello, Nature 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography.

Antibody variable domains with the desired binding specificities can be fused to immunoglobulin constant domain sequences. The fusion preferably is with an immunoglobulin heavy chain constant domain, comprising at least part of the hinge, C_{H}2 and C_{H}3 regions. It is preferred to have the first heavy chain constant region (C_{H}1) containing the site necessary for light chain binding present in at least one of the fusions. DNA encoding the immunoglobulin heavy chain fusions and, if desired, the immunoglobulin light chain, are inserted into separate expression vectors, and are co-transfected into a suitable host organism. Further details of generating bispecific antibodies can be found in Suresh et al., Methods in Enzymology 121:210 (1986).

Bispecific antibodies can be prepared as full-length antibodies or antibody fragments. Techniques for generating bispecific antibodies from antibody fragments have been described in the literature. For example, bispecific antibodies can be prepared using chemical linkage. In addition, Brennan et al., Science 229:81 (1985) describe a procedure wherein intact antibodies are proteolytically cleaved to generate F(ab′)₂ fragments.

Additionally, Fab′ fragments can be directly recovered from E. coli and chemically coupled to form bispecific antibodies (Shalaby et al., J. Exp. Med. 175:217-225 (1992)). These methods can be used in the production of a fully humanized bispecific antibody F(ab′)₂ molecule.

Antibodies with more than two valencies are also contemplated. For example, trispecific antibodies can be prepared (Tutt et al., J. Immunol. 147:60 (1991)).

Exemplary bispecific antibodies can bind to two different epitopes, at least one of which originates in a polypeptide of the invention. Alternatively, an anti-antigenic arm of an immunoglobulin molecule can be combined with an arm which binds to a triggering molecule on a leukocyte such as a T cell receptor molecule (e.g. CD2, CD3, CD28, or B7), or Fc receptors for IgG so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA.

Heteroconjugate antibodies are also within the scope of the present invention. Heteroconjugate antibodies are composed of two covalently joined antibodies. Such antibodies have, for example, been proposed to target immune cells to unwanted cells (U.S. Pat. No. 4,676,980). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies may comprise any type of variable region that provides for the association of the antibody with the polypeptides of IGSF9 or LIV-1. In this regard, the variable region may comprise or be derived from any type of mammal that can be induced to mount a humoral response and generate immunoglobulins against the desired tumor associated antigen. As such, the variable region of the modified antibodies may be, for example, of human, murine, non-human primate (e.g. cynomolgus monkeys, macaques, etc.) or lupine origin. In particularly preferred embodiments both the variable and constant regions of the modified immunoglobulins are human. In other selected embodiments the variable regions of compatible antibodies (usually derived from a non-human source) may be engineered or specifically tailored to improve the binding properties or reduce the immunogenicity of the molecule. In this respect, variable regions useful in the present invention may be humanized or otherwise altered through the inclusion of imported amino acid sequences.

By “humanized antibody” is meant an antibody derived from a non-human source, typically a murine antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. 81:6851-6855 (1984); Morrison et al., Adv. Immunol. 44:65-92 (1988); Verhoeyen et al., Science 239:1534-1536 (1988); Padlan, Molec. Immun. 28:489-498 (1991); Padlan, Molec. Immun. 31:169-217 (1994), and U.S. Pat. Nos. 5,585,089, 5,693,761 and 5,693,762 all of which are hereby incorporated by reference in their entirety.

Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)).

Methods for humanizing non-human antibodies are well known in the art. Generally, a humanized antibody has one or more amino acid residues introduced into it from a source that is non-human. These non-human amino acid residues are often referred to as “import” residues, which are typically taken from an “import” variable domain. Humanization can be essentially performed following the method of Winter and co-workers [Jones et al., Nature 321:522-525 (1986); Riechmann et al., Nature 332:323-327 (1988); Verhoeyen et al., Science 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA responses (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al., J. Mol. Biol. 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol. 151:2623 (1993)).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of humanized antibodies are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG₁ antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno. 7:33 (1993); U.S. Pat. Nos. 5,545,806, 5,569,825, 5,591,669 (all of GenPharm); 5,545,807; and WO 97/17852.

Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, K. S. and Chiswell, D. J., Current Opinion in Structural Biology 3:564-571 (1993). Several sources of V-gene segments can be used for phage display. Clackson et al., Nature 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

Those skilled in the art will appreciate that grafting the entire non-human variable domains onto human constant regions will produce “classic” chimeric antibodies. In the context of the present application the term “chimeric antibodies” will be held to mean any antibody wherein the immunoreactive region or site is obtained or derived from a first species and the constant region (which may be intact, partial or modified in accordance with this invention) is obtained from a second species. In preferred embodiments, the antigen binding region or site will be from a non-human source (e.g. mouse) and the constant region is human. While the immunogenic specificity of the variable region is not generally affected by its source, a human constant region is less likely to elicit an immune response from a human subject than would the constant region from a non-human source.

Preferably, the variable domains in both the heavy and light chains are altered by at least partial replacement of one or more CDRs and, if necessary, by partial framework region replacement and sequence changing. Although the CDRs may be derived from an antibody of the same class or even subclass as the antibody from which the framework regions are derived, it is envisaged that the CDRs will be derived from an antibody of different class and preferably from an antibody from a different species. It must be emphasized that it may not be necessary to replace all of the CDRs with the complete CDRs from the donor variable region to transfer the antigen binding capacity of one variable domain to another. Rather, it may only be necessary to transfer those residues that are necessary to maintain the activity of the antigen binding site. Given the explanations set forth in U.S. Pat. Nos. 5,585,089, 5,693,761 and 5,693,762, it will be well within the competence of those skilled in the art, either by carrying out routine experimentation or by trial and error testing to obtain a functional antibody with reduced immunogenicity.

Alterations to the variable region notwithstanding, those skilled in the art will appreciate that the modified antibodies of this invention will comprise antibodies, or immunoreactive fragments thereof, in which at least a fraction of one or more of the constant region domains has been deleted or otherwise altered so as to provide desired biochemical characteristics such as increased tumor localization or reduced serum half-life when compared with an antibody of approximately the same immunogenicity comprising a native or unaltered constant region. In preferred embodiments, the constant region of the modified antibodies will comprise a human constant region. Modifications to the constant region compatible with this invention comprise additions, deletions or substitutions of one or more amino acids in one or more domains. That is, the modified antibodies disclosed herein may comprise alterations or modifications to one or more of the three heavy chain constant domains (C_{H}1, C_{H}2 or C_{H}3) and/or to the light chain constant domain (C_{L}). As will be discussed in more detail below and shown in the examples, preferred embodiments of the invention comprise modified constant regions wherein one or more domains are partially or entirely deleted. In especially preferred embodiments the modified antibodies will comprise domain deleted constructs or variants wherein the entire C_{H}2 domain has been removed (ΔC_{H}2 constructs). In still other preferred embodiments the omitted constant region domain will be replaced by a short amino acid spacer (e.g. 10 residues) that provides some of the molecular flexibility typically imparted by the absent constant region.

Besides their configuration, it is known in the art that the constant region mediates several effector functions. For example, binding of the C1 component of complement to antibodies activates the complement system. Activation of complement is important in the opsonisation and lysis of cell pathogens. The activation of complement also stimulates the inflammatory response and may also be involved in autoimmune hypersensitivity. Further, antibodies bind to cells via the Fc region, with a Fc receptor site on the antibody Fc region binding to a Fc receptor (FcR) on a cell. There are a number of Fc receptors which are specific for different classes of antibody, including IgG (gamma receptors), IgE (eta receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. Although various Fc receptors and receptor sites have been studied to a certain extent, there is still much which is unknown about their location, structure and functioning.

While not limiting the scope of the present invention, it is believed that antibodies comprising constant regions modified as described herein provide for altered effector functions that, in turn, affect the biological profile of the administered antibody. For example, the deletion or inactivation (through point mutations or other means) of a constant region domain may reduce Fc receptor binding of the circulating modified antibody thereby increasing tumor localization. In other cases it may be that constant region modifications, consistent with this invention, moderate complement binding and thus reduce the serum half life and nonspecific association of a conjugated cytotoxin. Yet other modifications of the constant region may be used to eliminate disulfide linkages or oligosaccharide moieties that allow for enhanced localization due to increased antigen specificity or antibody flexibility. More generally, those skilled in the art will realize that antibodies modified as described herein may exert a number of subtle effects that may or may not be appreciated. Similarly, modifications to the constant region in accordance with this invention may easily be made using well known biochemical or molecular engineering techniques well within the purview of the skilled artisan.

It will be noted that the modified antibodies may be engineered to fuse the C_{H}3 domain directly to the hinge region of the respective modified antibodies. In other constructs it may be desirable to provide a peptide spacer between the hinge region and the modified C_{H}2 and/or C_{H}3 domains. For example, compatible constructs could be expressed wherein the C_{H}2 domain has been deleted and the remaining C_{H}3 domain (modified or unmodified) is joined to the hinge region with a 5-20 amino acid spacer. In this respect, one preferred spacer has the amino acid sequence IGKTISKKAK (SEQ ID NO:44). Such a spacer may be added, for instance, to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, it is preferable that any spacer added to the construct be relatively non-immunogenic or, even more preferably, omitted altogether if the desired biochemical qualities of the modified antibodies may be maintained.

Besides the deletion of whole constant region domains, it will be appreciated that the antibodies of the present invention may be provided by the partial deletion or substitution of a few or even a single amino acid. For example, the mutation of a single amino acid in selected areas of the C_{H}2 domain may be enough to substantially reduce Fc binding and thereby increase tumor localization. Similarly, it may be desirable to simply delete that part of one or more constant region domains that control the effector function (e.g. complement CLQ binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g. Fc binding) while substantially maintaining the configuration and immunogenic profile of the modified antibody. Yet other preferred embodiments may comprise the addition of one or more amino acids to the constant region to enhance desirable characteristics such as effector function or provide for more cytotoxin or carbohydrate attachment. In such embodiments it may be desirable to insert or replicate specific sequences derived from selected constant region domains.

In particularly preferred embodiments the cloned variable region genes are inserted into an expression vector along with the heavy and light chain constant region genes (preferably human) modified as discussed above. Preferably, this is effected using a proprietary expression vector of IDEC, Inc., referred to as NEOSPLA. This vector contains the cytomegalovirus promoter/enhancer, the mouse beta globin major promoter, the SV40 origin of replication, the bovine growth hormone polyadenylation sequence, neomycin phosphotransferase exon 1 and exon 2, the dihydrofolate reductase gene and leader sequence. As seen in the examples below, this vector has been found to result in very high level expression of antibodies upon incorporation of variable and constant region genes, transfection in CHO cells, followed by selection in G418 containing medium and methotrexate amplification. This vector system is substantially disclosed in commonly assigned U.S. Pat. Nos. 5,736,137 and 5,658,570, each of which is incorporated by reference in its entirety herein. This system provides for high expression levels, i.e., >30 pg/cell/day.

In other preferred embodiments the modified antibodies of this invention may be expressed using polycistronic constructs such as those disclosed in U.S. provisional application No. 60/331,481 filed Nov. 16, 2001 and incorporated herein in its entirety. In these novel expression systems, multiple gene products of interest such as heavy and light chains of antibodies may be produced from a single polycistronic construct. These systems advantageously use an internal ribosome entry site (IRES) to provide relatively high levels of modified antibodies in eukaryotic host cells. Compatible IRES sequences are disclosed in U.S. Pat. No. 6,193,980 which is also incorporated herein. Those skilled in the art will appreciate that such expression systems may be used to effectively produce the full range of modified antibodies disclosed in this application.

More generally, once the vector or DNA sequence containing a polypeptide of the invention, such as a modified antibody, has been prepared, the expression vector may be introduced into an appropriate host cell. That is, the host cells may be transformed. Introduction of the plasmid into the host cell can be accomplished by various techniques well known to those of skill in the art. These include, but are not limited to, transfection (including electrophoresis and electroporation), protoplast fusion, calcium phosphate precipitation, cell fusion with enveloped DNA, microinjection, and infection with intact virus. See, Ridgway, A. A. G. “Mammalian Expression Vectors” Chapter 24.2, pp. 470-472 Vectors, Rodriguez and Denhardt, Eds. (Butterworths, Boston, Mass. 1988). Most preferably, plasmid introduction into the host is via electroporation. The transformed cells are grown under conditions appropriate to the production of the light chains and heavy chains, and assayed for heavy and/or light chain protein synthesis. Exemplary assay techniques include enzyme-linked immunosorbent assay (ELISA), radioimmunoassay (RIA), or flourescence-activated cell sorter analysis (FACS), immunohistochemistry and the like.

As used herein, the term “transformation” shall be used in a broad sense to refer to any introduction of DNA into a recipient host cell that changes the genotype and consequently results in a change in the recipient cell.

Along those same lines, “host cells” refers to cells that have been transformed with vectors constructed using recombinant DNA techniques and containing at least one heterologous gene. As defined herein, the antibody or modification thereof produced by a host cell is by virtue of this transformation. In descriptions of processes for isolation of antibodies from recombinant hosts, the terms “cell” and “cell culture” are used interchangeably to denote the source of antibody unless it is clearly specified otherwise. In other words, recovery of antibody from the “cells” may mean either from spun down whole cells, or from the cell culture containing both the medium and the suspended cells.

The host cell line used for protein expression is most preferably of mammalian origin; those skilled in the art are credited with ability to preferentially determine particular host cell lines which are best suited for the desired gene product to be expressed therein. Exemplary host cell lines include, but are not limited to, DG44 and DUXB11 (Chinese Hamster Ovary lines, DHFR minus), HELA (human cervical carcinoma), CVI (monkey kidney line), COS (a derivative of CVI with SV40 T antigen), R1610 (Chinese hamster fibroblast) BALBC/3T3 (mouse fibroblast), HAK (hamster kidney line), SP2/O (mouse myeloma), P3.times.63-Ag3.653 (mouse myeloma), BFA-1c1BPT (bovine endothelial cells), RAJI (human lymphocyte) and 293 (human kidney). CHO cells are particularly preferred. Host cell lines are typically available from commercial services, the American Tissue Culture Collection or from published literature.

In vitro production allows scale-up to give large amounts of the desired antibodies. Techniques for mammalian cell cultivation under tissue culture conditions are known in the art and include homogeneous suspension culture, e.g. in an airlift reactor or in a continuous stirrer reactor, or immobilized or entrapped cell culture, e.g. in hollow fibers, microcapsules, on agarose microbeads or ceramic cartridges. For isolation of the modified antibodies, the immunoglobulins in the culture supernatants are first concentrated, e.g. by precipitation with ammonium sulphate, dialysis against hygroscopic material such as PEG, filtration through selective membranes, or the like. If necessary and/or desired, the concentrated antibodies are purified by the customary chromatography methods, for example gel filtration, ion-exchange chromatography, chromatography over DEAE-cellulose or (immuno-)affinity chromatography.

The modified immunoglobulin genes and/or polypeptides of the invention can also be expressed in non-mammalian cells such as bacteria or yeast. In this regard, it will be appreciated that various unicellular non-mammalian microorganisms such as bacteria can also be transformed; i.e. those capable of being grown in cultures or fermentation. Bacteria, which are susceptible to transformation, include members of the enterobacteriaceae, such as strains of Escherichia coli; Salmonella; Bacillaceae, such as Bacillus subtilis; Pneumococcus; Streptococcus, and Haemophilus influenzae. It will further be appreciated that, when expressed in bacteria, the immunoglobulin heavy chains and light chains typically become part of inclusion bodies. The chains then must be isolated, purified and then assembled into functional immunoglobulin molecules.

In addition to prokaryotes, eukaryotic microbes may also be used. Saccharomyces cerevisiae, or common baker's yeast, is the most commonly used among eukaryotic microorganisms although a number of other strains are commonly available.

For expression in Saccharomyces, the plasmid YRp7, for example, (Stinchcomb et al., Nature 282:39 (1979); Kingsman et al., Gene 7:141 (1979); Tschemper et al., Gene 10:157 (1980)) is commonly used. This plasmid already contains the trp1 gene which provides a selection marker for a mutant strain of yeast lacking the ability to grow in tryptophan, for example ATCC No. 44076 or PEP4-1 (Jones, Genetics 85:12 (1977)). The presence of the trp1 lesion as a characteristic of the yeast host cell genome then provides an effective environment for detecting transformation by growth in the absence of tryptophan.

Regardless of how clinically useful quantities are obtained, the modified antibodies of the present invention may be used in any one of a number of conjugated (i.e. an immunoconjugate) or unconjugated forms. In particular, the antibodies of the present invention may be conjugated to cytotoxins such as radioisotopes, therapeutic agents, cytostatic agents, biological toxins or prodrugs. Alternatively, the modified antibodies of this invention may be used in a nonconjugated or “naked” form to harness the subject's natural defense mechanisms including complement-dependent cytotoxicity (CDC) and antibody dependent cellular toxicity (ADCC) to eliminate the malignant cells. In particularly preferred embodiments, the modified antibodies may be conjugated to radioisotopes, such as ⁹⁰Y, ¹²⁵I, ¹³¹I, ¹²³I, ¹¹¹In, ¹⁰⁵Rh, ¹⁵³Sm, ⁶⁷Cu, ⁶⁷Ga, ¹⁶⁶Ho, ¹⁷⁷Lu, ¹⁸⁶Re and ¹⁸⁸Re using anyone of a number of well known chelators or direct labeling. In other embodiments, the disclosed compositions may comprise modified antibodies coupled to drugs, prodrugs or biological response modifiers such as methotrexate, adriamycin, and lymphokines such as interferon. Still other embodiments of the present invention comprise the use of modified antibodies conjugated to specific biotoxins such as ricin or diptheria toxin. In yet other embodiments the modified antibodies may be complexed with other immunologically active ligands (e.g. antibodies or fragments thereof) wherein the resulting molecule binds to both the neoplastic cell and an effector cell such as a T cell. The selection of which conjugated or unconjugated modified antibody to use will depend of the type and stage of cancer, use of adjunct treatment (e.g., chemotherapy or external radiation) and patient condition. It will be appreciated that one skilled in the art could readily make such a selection in view of the teachings herein.

As used herein, “a cytotoxin or cytotoxic agent” means any agent that is detrimental to the growth and proliferation of cells and may act to reduce, inhibit or destroy a malignancy when exposed thereto. Exemplary cytotoxins include, but are not limited to, radionuclides, biotoxins, cytostatic or cytotoxic therapeutic agents, prodrugs, immunologically active ligands and biological response modifiers such as cytokines. As will be discussed in more detail below, radionuclide cytotoxins are particularly preferred for use in this invention. However, any cytotoxin that acts to retard or slow the growth of malignant cells or to eliminate malignant cells and may be associated with the modified antibodies disclosed herein is within the purview of the present invention.

It will be appreciated that, in previous studies, anti-tumor antibodies labeled with isotopes have been used successfully to destroy cells in solid tumors as well as lymphomas/leukemias in animal models, and in some cases in humans. The radionuclides act by producing ionizing radiation which causes multiple strand breaks in nuclear DNA, leading to cell death. The isotopes used to produce therapeutic conjugates typically produce high energy α-, γ- or β-particles which have a therapeutically effective path length. Such radionuclides kill cells to which they are in close proximity, for example neoplastic cells to which the conjugate has attached or has entered. They generally have little or no effect on non-localized cells. Radionuclides are essentially non-immunogenic.

With respect to the use of radiolabeled conjugates in conjunction with the present invention, the modified antibodies may be directly labeled (such as through iodination) or may be labeled indirectly through the use of a chelating agent. As used herein, the phrases “indirect labeling” and “indirect labeling approach” both mean that a chelating agent is covalently attached to an antibody and at least one radionuclide is associated with the chelating agent. Such chelating agents are typically referred to as bifunctional chelating agents as they bind both the polypeptide and the radioisotope. Particularly preferred chelating agents comprise 1-isothiocycmatobenzyl-3-methyldiothelene triaminepentaacetic acid (“Mx-DTPA”) and cyclohexyl diethylenetriamine pentaacetic acid (“CHX-DTPA”) derivatives. Other chelating agents comprise P-DOTA and EDTA derivatives. Particularly preferred radionuclides for indirect labeling include ¹¹¹In and ⁹⁰Y.

As used herein, the phrases “direct labeling” and “direct labeling approach” both mean that a radionuclide is covalently attached directly to an antibody (typically via an amino acid residue). More specifically, these linking technologies include random labeling and site-directed labeling. In the latter case, the labeling is directed at specific sites on the dimer or tetramer, such as the N-linked sugar residues present only on the Fc portion of the conjugates. Further, various direct labeling techniques and protocols are compatible with this invention. For example, Technetium-99m labeled antibodies may be prepared by ligand exchange processes, by reducing pertechnate (TcO₄⁻) with stannous ion solution, chelating the reduced technetium onto a Sephadex column and applying the antibodies to this column, or by batch labeling techniques, e.g. by incubating pertechnate, a reducing agent such as SnCl₂, a buffer solution such as a sodium-potassium phthalate-solution, and the antibodies. In any event, preferred radionuclides for directly labeling antibodies are well known in the art and a particularly preferred radionuclide for direct labeling is ¹³¹I, covalently attached via tyrosine residues. Modified antibodies according to the invention may be derived, for example, with radioactive sodium or potassium iodide and a chemical oxidizing agent, such as sodium hypochlorite, chloramine T or the like, or an enzymatic oxidizing agent, such as lactoperoxidase, glucose oxidase and glucose. However, for the purposes of the present invention, the indirect labeling approach is particularly preferred.

Patents relating to chelators and chelator conjugates are known in the art. For instance, U.S. Pat. No. 4,831,175 of Gansow is directed to polysubstituted diethylenetriaminepentaacetic acid chelates and protein conjugates containing the same, and methods for their preparation. U.S. Pat. Nos. 5,099,069, 5,246,692, 5,286,850, 5,434,287 and 5,124,471 of Gansow also relate to polysubstituted DTPA chelates. These patents are incorporated herein in their entirety. Other examples of compatible metal chelators are ethylenediaminetetraacetic acid (EDTA), diethylenetriaminepentaacetic acid (DPTA), 1,4,8,11-tetraazatetradecane, 1,4,8,11-tetraazatetradecane-1,4,8,11-tetraacetic acid, 1-oxa-4,7,12,15-tetraazaheptadecane-4,7,12,15-tetraacetic acid, or the like. Cyclohexyl-DTPA or CHX-DTPA is particularly preferred and is exemplified extensively below. Still other compatible chelators, including those yet to be discovered, may easily be discerned by a skilled artisan and are clearly within the scope of the present invention.

Compatible chelators, including the specific bifunctional chelator used to facilitate chelation in co-pending application Ser. Nos. 08/475,813, 08/475,815 and 08/478,967, are preferably selected to provide high affinity for trivalent metals, exhibit increased tumor-to-non-tumor ratios and decreased bone uptake as well as greater in vivo retention of radionuclide at target sites, i.e., B-cell lymphoma tumor sites. However, other bifunctional chelators that may or may not possess all of these characteristics are known in the art and may also be beneficial in tumor therapy.

It will also be appreciated that, in accordance with the teachings herein, modified antibodies may be conjugated to different radiolabels for diagnostic and therapeutic purposes. To this end the aforementioned co-pending applications, herein incorporated by reference in their entirety, disclose radiolabeled therapeutic conjugates for diagnostic “imaging” of tumors before administration of therapeutic antibody. “In2B8” conjugate comprises a murine monoclonal antibody, 2B8, specific to human CD20 antigen, that is attached to ¹¹¹In via a bifunctional chelator, i.e., MX-DTPA (diethylenetriaminepentaacetic acid), which comprises a 1:1 mixture of 1-isothiocyanatobenzyl-3-methyl-DTPA and 1-methyl-3-isothiocyanatobenzyl-DTPA. ¹¹¹In is particularly preferred as a diagnostic radionuclide because between about 1 to about 10 mCi can be safely administered without detectable toxicity; and the imaging data is generally predictive of subsequent ⁹⁰Y-labeled antibody distribution. Most imaging studies utilize 5 mCi ¹¹¹In-labeled antibody, because this dose is both safe and has increased imaging efficiency compared with lower doses, with optimal imaging occurring at three to six days after antibody administration. See, for example, Murray, J. Nuc. Med. 26: 3328 (1985) and Carraguillo et al., J. Nuc. Med. 26: 67 (1985).

As indicated above, a variety of radionuclides are applicable to the present invention and those skilled in the art are credited with the ability to readily determine which radionuclide is most appropriate under various circumstances. For example, ¹³¹I is a well known radionuclide used for targeted immunotherapy. However, the clinical usefulness of ¹³¹I can be limited by several factors including: eight-day physical half-life; dehalogenation of iodinated antibody both in the blood and at tumor sites; and emission characteristics (e.g., large gamma component) which can be suboptimal for localized dose deposition in tumor. With the advent of superior chelating agents, the opportunity for attaching metal chelating groups to proteins has increased the opportunities to utilize other radionuclides such as ¹¹¹In and ⁹⁰Y. ⁹⁰Y provides several benefits for utilization in radioimmunotherapeutic applications: the 64 hour half-life of ⁹⁰Y is long enough to allow antibody accumulation by tumor and, unlike e.g., ¹³¹I, ⁹⁰Y is a pure beta emitter of high energy with no accompanying gamma irradiation in its decay, with a range in tissue of 100 to 1,000 cell diameters. Furthermore, the minimal amount of penetrating radiation allows for outpatient administration of ⁹⁰Y-labeled antibodies. Additionally, internalization of labeled antibody is not required for cell killing, and the local emission of ionizing radiation should be lethal for adjacent tumor cells lacking the target antigen.

Effective single treatment dosages (i.e., therapeutically effective amounts) of ⁹⁰Y-labeled modified antibodies range from between about 5 and about 75 mCi, more preferably between about 10 and about 40 mCi. Effective single treatment non-marrow ablative dosages of ¹³¹I-labeled antibodies range from between about 5 and about 70 mCi, more preferably between about 5 and about 40 mCi. Effective single treatment ablative dosages (i.e., may require autologous bone marrow transplantation) of ¹³¹I-labeled antibodies range from between about 30 and about 600 mCi, more preferably between about 50 and less than about 500 mCi. In conjunction with a chimeric antibody, owing to the longer circulating half life vis-a-vis murine antibodies, an effective single treatment non-marrow ablative dosages of iodine-131 labeled chimeric antibodies range from between about 5 and about 40 mCi, more preferably less than about 30 mCi. Imaging criteria for, e.g., the ¹¹¹In label, are typically less than about 5 mCi.

While a great deal of clinical experience has been gained with ¹³¹I and ⁹⁰Y, other radiolabels are known in the art and have been used for similar purposes. Still other radioisotopes are used for imaging. For example, additional radioisotopes which are compatible with the scope of this invention include, but are not limited to, ¹²³I, ¹²⁵I, ³²P, ⁵⁷Co, ⁶⁴Cu, ⁶⁷Cu, ⁷⁷Br, ⁸¹Rb, ⁸¹Kr, ⁸⁷Sr, ¹¹³In, ¹²⁷Cs, ¹²⁹Cs, ¹³²I, ¹⁹⁷Hg, ²⁰³Pb, ²⁰⁶Bi, ¹⁷⁷Lu, ¹⁸⁶Re, ²¹²Pb, ²¹²Bi, ⁴⁷Sc, ¹⁰⁵Rh, ¹⁰⁹Pd, ¹⁵³Sm, ¹⁸⁸Re, ¹⁹⁹Au, ²²⁵Ac, ²¹¹At, and ²¹³Bi. In this respect alpha, gamma and beta emitters are all compatible with in this invention. Further, in view of this disclosure it is submitted that one skilled in the art could readily determine which radionuclides are compatible with a selected course of treatment without undue experimentation. To this end, additional radionuclides which have already been used in clinical diagnosis include ¹²⁵I, ¹²³I, ⁹⁹Tc, ⁴³K, ⁵²Fe, ⁶⁷Ga, ⁶⁸Ga, as well as ¹¹¹In. Antibodies have also been labeled with a variety of radionuclides for potential use in targeted immunotherapy Peirersz et al. Immunol. Cell Biol. 65: 111-125 (1987). These radionuclides include ¹⁸⁸Re and ¹⁸⁶Re as well as ¹⁹⁹Au and ⁶⁷Cu to a lesser extent. U.S. Pat. No. 5,460,785 provides additional data regarding such radioisotopes and is incorporated herein by reference.

In addition to radionuclides, the modified antibodies of the present invention may be conjugated to, or associated with, any one of a number of biological response modifiers, pharmaceutical agents, toxins or immunologically active ligands. Those skilled in the art will appreciate that these non-radioactive conjugates may be assembled using a variety of techniques depending on the selected cytotoxin. For example, conjugates with biotin are prepared e.g. by reacting the modified antibodies with an activated ester of biotin such as the biotin N-hydroxysuccinimide ester. Similarly, conjugates with a fluorescent marker may be prepared in the presence of a coupling agent, e.g. those listed above, or by reaction with an isothiocyanate, preferably fluorescein-isothiocyanate. Conjugates of the chimeric antibodies of the invention with cytostatic/cytotoxic substances and metal chelates are prepared in an analogous manner.

Preferred agents for use in the present invention are cytotoxic drugs, particularly those which are used for cancer therapy. Such drugs include, in general, cytostatic agents, alkylating agents, antimetabolites, anti-proliferative agents, tubulin binding agents, hormones and hormone antagonists, and the like. Exemplary cytostatics that are compatible with the present invention include alkylating substances, such as mechlorethamine, triethylenephosphoramide, cyclophosphamide, ifosfamide, chlorambucil, busulfan, melphalan or triaziquone, also nitrosourea compounds, such as carmustine, lomustine, or semustine. Other preferred classes of cytotoxic agents include, for example, the anthracycline family of drugs, the vinca drugs, the mitomycins, the bleomycins, the cytotoxic nucleosides, the pteridine family of drugs, diynenes, and the podophyllotoxins. Particularly useful members of those classes include, for example, adriamycin, carminomycin, daunorubicin (daunomycin), doxorubicin, aminopterin, methotrexate, methopterin, mithramycin, streptonigrin, dichloromethotrexaite, mitomycin C, actinomycin-D, porfiromycin, 5-fluorouracil, floxuridine, ftorafur, 6-mercaptopurine, cytarabine, cytosine arabinoside, podophyllotoxin, or podophyllotoxin derivatives such as etoposide or etoposide phosphate, melphalan, vinblastine, vincristine, leurosidine, vindesine, leurosine and the like. Still other cytotoxins that are compatible with the teachings herein include taxol, taxane, cytochalasin B, gramicidin D, ethidium bromide, emetine, tenoposide, colchicin, dihydroxy anthracin dione, mitoxantrone, procaine, tetracaine, lidocaine, propranolol, and puromycin and analogs or homologs thereof. Hormones and hormone antagonists, such as corticosteroids, e.g. prednisone, progestins, e.g. hydroxyprogesterone or medroprogesterone, estrogens, e.g. diethylstilbestrol, antiestrogens, e.g. tamoxifen, androgens, e.g. testosterone, and aromatase inhibitors, e.g. aminogluthetimide are also compatible with the teachings herein. As noted previously, one skilled in the art may make chemical modifications to the desired compound in order to make reactions of that compound more convenient for purposes of preparing conjugates of the invention.

One example of particularly preferred cytotoxins comprises members or derivatives of the enediyne family of anti-tumor antibiotics, including calicheamicin, esperamicins or dynemicins. These toxins are extremely potent and act by cleaving nuclear DNA, leading to cell death. Unlike protein toxins which can be cleaved in vivo to give many inactive but immunogenic polypeptide fragments, toxins such as calicheamicin, esperamicins and other enediynes are small molecules which are essentially non-immunogenic. These non-peptide toxins are chemically-linked to the dimers or tetramers by techniques which have been previously used to label monoclonal antibodies and other molecules. These linking technologies include site-specific linkage via the N-linked sugar residues present only on the Fc portion of the conjugates. Such site-directed linking methods have the advantage of reducing the possible effects of linkage on the binding properties of the conjugate.

As previously alluded to, compatible cytotoxins may comprise a prodrug. As used herein, the term “prodrug” refers to a precursor or derivative form of a pharmaceutically active substance that is less cytotoxic to tumor cells compared to the parent drug and is capable of being enzymatically activated or converted into the more active parent form. Prodrugs compatible with the invention include, but are not limited to, phosphate-containing prodrugs, thiophosphate-containing prodrugs, sulfate containing prodrugs, peptide containing prodrugs, β-lactam-containing prodrugs, optionally substituted phenoxyacetamide-containing prodrugs or optionally substituted phenylacetamide-containing prodrugs, 5-fluorocytosine and other 5-fluorouridine prodrugs that can be converted to the more active cytotoxic free drug. Further examples of cytotoxic drugs that can be derivatized into a prodrug form for use in the present invention comprise those chemotherapeutic agents described above.

Among other cytotoxins, it will be appreciated that the antibody can also be associated with a biotoxin such as ricin subunit A, abrin, diptheria toxin, botulinum, cyanginosins, saxitoxin, shigatoxin, tetanus, tetrodotoxin, trichothecene, verrucologen or a toxic enzyme. Preferably, such constructs will be made using genetic engineering techniques that allow for direct expression of the antibody-toxin construct. Other biological response modifiers that may be associated with the modified antibodies of the present invention comprise cytokines such as lymphokines and interferons. Moreover, as indicated above, similar constructs may also be used to associate immunologically active ligands (e.g. antibodies or fragments thereof) with the modified antibodies of the present invention. Preferably, these immunologically active ligands would be directed to antigens on the surface of immunoactive effector cells. In these cases, the constructs could be used to bring effector cells, such as T cells or NK cells, in close proximity to the neoplastic cells bearing a tumor associated antigen thereby provoking the desired immune response. In view of this disclosure it is submitted that one skilled in the art could readily form such constructs using conventional techniques.

Another class of compatible cytotoxins that may be used in conjunction with the disclosed modified antibodies are radiosensitizing drugs that may be effectively directed to tumor cells. Such drugs enhance the sensitivity to ionizing radiation, thereby increasing the efficacy of radiotherapy. An antibody conjugate internalized by the tumor cell would deliver the radiosensitizer nearer the nucleus where radiosensitization would be maximal. The unbound radiosensitizer linked modified antibodies would be cleared quickly from the blood, localizing the remaining radiosensitization agent in the target tumor and providing minimal uptake in normal tissues. After rapid clearance from the blood, adjunct radiotherapy would be administered in one of three ways: 1.) external beam radiation directed specifically to the tumor, 2.) radioactivity directly implanted in the tumor or 3.) systemic radioimmunotherapy with the same targeting antibody. A potentially attractive variation of this approach would be the attachment of a therapeutic radioisotope to the radiosensitized immunoconjugate, thereby providing the convenience of administering to the patient a single drug.

Whether or not the disclosed antibodies are used in a conjugated or unconjugated form, it will be appreciated that a major advantage of the present invention is the ability to use these antibodies in myelosuppressed patients, especially those who are undergoing, or have undergone, adjunct therapies such as radiotherapy or chemotherapy. That is, the beneficial delivery profile (i.e. relatively short serum dwell time and enhanced localization) of the modified antibodies makes them particularly useful for treating patients that have reduced red marrow reserves and are sensitive to myelotoxicity. In this regard, the unique delivery profile of the modified antibodies make them very effective for the administration of radiolabeled conjugates to myelosuppressed cancer patients. As such, the modified antibodies are useful in a conjugated or unconjugated form in patients that have previously undergone adjunct therapies such as external beam radiation or chemotherapy. In other preferred embodiments, the modified antibodies (again in a conjugated or unconjugated form) may be used in a combined therapeutic regimen with chemotherapeutic agents. Those skilled in the art will appreciate that such therapeutic regimens may comprise the sequential, simultaneous, concurrent or coextensive administration of the disclosed antibodies and one or more chemotherapeutic agents. Particularly preferred embodiments of this aspect of the invention will comprise the administration of a radiolabeled antibody.

While the modified antibodies may be administered as described immediately above, it must be emphasized that in other embodiments conjugated and unconjugated modified antibodies may be administered to otherwise healthy cancer patients as a first line therapeutic agent. In such embodiments the modified antibodies may be administered to patients having neoplasia and/or to patients that have not, and are not, undergoing adjunct therapies such as external beam radiation or chemotherapy.

Polypeptides of the Invention

The invention further provides isolated IGSF9 or LIV-1 polypeptides having the amino acid sequence in FIGS. 1B, 9F, 21B, or 22B (SEQ ID NOS:2, 4, 6, 8, 22-27, or 29), or a peptide or polypeptide comprising a portion of the above polypeptides. The terms “peptide” and “oligopeptide” are considered synonymous (as is commonly recognized) and each term can be used interchangeably as the context requires to indicate a chain of at least to amino acids coupled by peptidyl linkages. The word “polypeptide” is used herein for chains containing more than ten amino acid residues. All oligopeptide and polypeptide formulas or sequences herein are written from left to right and in the direction from amino terminus to carboxy terminus.

It will be recognized in the art that some amino acid sequences of the IGSF9 or LIV-1 polypeptides can be varied without significant effect of the structure or function of the protein. If such differences in the sequences are contemplated, it should be remembered that there will be critical areas on the proteins which determine activity. In general, it is possible to replace residues that form the tertiary structure, provided that residues performing a similar function are used. In other instances, the type of residue may be completely unimportant if the alteration occurs at a non-critical region of the protein.

Thus, the invention further includes variations of the IGSF9 or LIV-1 polypeptides that include regions of the IGSF9 or LIV-1 proteins such as the protein portions discussed below. Such mutants include deletions, insertions, inversions, repeats, and type substitutions (for example, substituting one hydrophilic residue for another, but not strongly hydrophilic for strongly hydrophobic as a rule). Small changes or such “neutral” amino acid substitutions will generally have little effect on activity.

Typically seen as conservative substitutions are the replacements, one for another, among the aliphatic amino acids Ala, Val, Leu and Ile; interchange of the hydroxyl residues Ser and Thr, exchange of the acidic residues Asp and Glu, substitution between the amide residues Asn and Gln, exchange of the basic residues Lys and Arg and replacements among the aromatic residues Phe, Tyr.

As indicated in detail above, further guidance concerning which amino acid changes are likely to be phenotypically silent (i.e., are not likely to have a significant deleterious effect on a function) can be found in Bowie, J. U., et al., “Deciphering the Message in Protein Sequences: Tolerance to Amino Acid Substitutions,” Science 247:1306-1310 (1990).

Thus, the fragment, derivative or analog of the polypeptides of FIGS. 1B, 9B, 9D, 9F, 21B, or 22B (SEQ ID NOS:2, 4, 6, 8, 22-27, or 29), may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide, such as an IgG Fc fusion region peptide or leader or secretory sequence or a sequence which is employed for purification of the mature polypeptide or a proprotein sequence. Such fragments, derivatives and analogs are deemed to be within the scope of those skilled in the art from the teachings herein.

Amino acids in the IGSF9 or LIV-1 proteins of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity. Sites that are critical for ligand-receptor binding can also be determined by structural analysis such as crystallization, nuclear magnetic resonance or photoaffinity labeling (Smith et al, J. Mol. Biol. 224:899-904 (1992) and de Vos et al. Science 255:306-312 (1992)).

The polypeptides of the present invention are preferably provided in an isolated form. By “isolated polypeptide” is intended a polypeptide removed from its native environment. Thus, a polypeptide produced and/or contained within a recombinant host cell is considered isolated for purposes of the present invention. Also intended as an “isolated polypeptide” are polypeptides that have been purified, partially or substantially, from a recombinant host cell.

A variety of methodologies known in the art can be utilized to obtain any one of the isolated polypeptides of the present invention. At the simplest level, the amino acid sequence can be synthesized using commercially available peptide synthesizers. The synthetically-constructed protein sequences, by virtue of sharing primary, secondary or tertiary structural and/or conformational characteristics with proteins may possess biological properties in common therewith, including protein activity. This technique is particularly useful in producing small peptides and fragments of larger polypeptides. Fragments are useful, for example, in generating antibodies against the native polypeptides. Thus, they may be employed as biologically active or immunological substitutes for natural, purified proteins in screening of therapeutic compounds and in immunological processes for the development of antibodies.

The polypeptides of the present invention can alternatively be purified from cells that have been altered to express the desired polypeptide. As used herein, a cell is said to be altered for expression of a desired polypeptide when the cell, through genetic manipulation, is made to produce a polypeptide which it normally does not produce or which the cell normally produces at a lower level. One skilled in the art can readily adapt procedures for introducing and expressing either recombinant or synthetic sequences into eukaryotic or prokaryotic cells in order to generate a cell that produces one of the polypeptides of the present invention. These include, inter alia, those plasmids and host cells described above. For example, a recombinantly produced version of either the IGSF9 or LIV-1 polypeptides can be substantially purified by the one-step method described in Smith and Johnson, Gene 67:31-40 (1988).

The IGSF9 or LIV-1 polypeptides of the present invention include the polypeptides including the leader; the mature polypeptide minus the leader (i.e., the mature protein); a polypeptide comprising amino acids from about 21 to about 718 in FIG. 1B (SEQ ID NO:2); a polypeptide comprising amino acids from about 1 to about 1179 in FIG. 9F (SEQ ID NO:8); a polypeptide comprising amino acids from about 21 to about 1179 in FIG. 9F (SEQ ID NO:8); a polypeptide comprising the sequence shown in SEQ ID NOS:4, 6, 22-27; a polypeptide comprising amino acids from about 28 to about 317 in FIG. 22B (SEQ ID NO:29); a polypeptide comprising amino acids from about 373 to about 417 in FIG. 22B (SEQ ID NO:29); a polypeptide comprising amino acids from about 674 to about 678 in FIG. 22B (SEQ ID NO:29); a polypeptide comprising amino acids from about 742 to about 749 in FIG. 22B (SEQ ID NO:29); as well as polypeptides which are at least 80% identical, more preferably at least 90% or 95% identical, still more preferably at least 96%, 97%, 98% or 99% identical to the polypeptides described above and also include portions of such polypeptides with at least 30 amino acids and more preferably at least 50 amino acids.

By “% similarity” for two polypeptides is intended a similarity score produced by comparing the amino acid sequences of the two polypeptides using the Bestfit program (Wisconsin Sequence Analysis Package, Version 8 for Unix, Genetics Computer Group, University Research Park, 575 Science Drive, Madison, Wis. 53711) and the default settings for determining similarity. Bestfit uses the local homology algorithm of Smith and Waterman (Advances in Applied Mathematics 2: 482-489, 1981) to find the best segment of similarity between two sequences.

By a polypeptide having an amino acid sequence at least, for example, 95% “identical” to a reference amino acid sequence of either an IGSF9 or LIV-1 polypeptide is intended that the amino acid sequence of the polypeptide is identical to the reference sequence except that the polypeptide sequence may include up to five amino acid alterations per each 100 amino acids of the reference amino acid of the IGSF9 or LIV-1 polypeptides. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 90%, 95%, 96%, 97%, 98% or 99% identical to, for instance, the amino acid sequence shown in FIGS. 1B and 22B (SEQ ID NOS:2 and 29) can be determined conventionally using known computer programs such the Bestfit program (Wisconsin Sequence Analysis Package, Version 8 for Unix, Genetics Computer Group, University Research Park, 575 Science Drive, Madison, Wis. 53711. When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

The polypeptides of the present invention are useful as a molecular weight marker on SDS-PAGE gels or on molecular sieve gel filtration columns using methods well known to those of skill in the art.

The purified polypeptides can be used in in vitro binding assays which are well known in the art to identify molecules which bind to the polypeptides. These molecules include, but are not limited to, for example, small molecules, molecules from combinatorial libraries, antibodies or other proteins.

In addition, the peptides of the invention or molecules capable of binding to the peptides may be complexed with toxins, e.g. ricin or cholera, or with other compounds that are toxic to cells. The toxin-binding molecule complex is then targeted to a tumor or other cell by specificity of the binding molecule for the polypeptides of FIGS. 1B, 9B, 9D, 9F, 21B, or 22B (SEQ ID NOS:2, 4, 6, 8, 22-27, or 29).

As described in detail previously, the polypeptides of the present invention can be used to raise polyclonal and monoclonal antibodies, which are useful in diagnostic assays for detecting IGSF9 or LIV-1 protein expression as described below or as agonists and antagonists capable of enhancing or inhibiting IGSF9 or LIV-1 protein function. Further, such polypeptides can be used in the yeast two-hybrid system to “capture” IGSF9 or LIV-1 protein binding proteins which are also candidate agonist and antagonist according to the present invention. The yeast two hybrid system is described in Fields and Song, Nature 340:245-246 (1989).

Polynucleotides of the Invention

The present invention also provides isolated nucleic acid molecules comprising polynucleotides encoding the polypeptides of IGSF9 or LIV-1 described above.

Unless otherwise indicated, each “nucleotide sequence” set forth herein is presented as a sequence of deoxyribonucleotides (abbreviated A, G, C and T). However, by “nucleotide sequence” of a nucleic acid molecule or polynucleotide is intended, for a DNA molecule or polynucleotide, a sequence of deoxyribonucleotides, and for an RNA molecule or polynucleotide, the corresponding sequence of ribonucleotides (A, G, C and U) where each thymidine deoxynucleotide (T) in the specified deoxynucleotide sequence is replaced by the ribonucleotide uridine (U). For instance, reference to an RNA molecule having the sequence of SEQ ID NO:1 set forth using deoxyribonucleotide abbreviations is intended to indicate an RNA molecule having a sequence in which each deoxynucleotide A, G or C of SEQ ID NO:1 has been replaced by the corresponding ribonucleotide A, G or C, and each deoxynucleotide T has been replaced by a ribonucleotide U.

Using the information provided herein, such as the nucleotide sequence in FIGS. 1A, 9A, 9C, 9E, 9H, 21A, and 22A, a nucleic acid molecule of the present invention encoding either an IGSF9-or LIV-1 polypeptide may be obtained using standard cloning and screening procedures, such as those for cloning cDNAs using mRNA as starting material. The isolated nucleic acids may also be cloned in vectors and propagated in host cells as described above and well known in the art.

The determined nucleotide sequence of IGSF9 in FIG. 1A contains an open reading frame encoding a protein of about 1163 amino acid residues with an initiation codon at position 1 of the nucleotide sequence shown in FIGS. 1A-1B (SEQ ID NOS:1-2), and a predicted leader sequence of about 20 amino acid residues. The amino acid sequence of the predicted IGSF9 protein further contains an extracellular domain from about amino acid 21 to about amino acid 718, as shown in FIG. 1B.

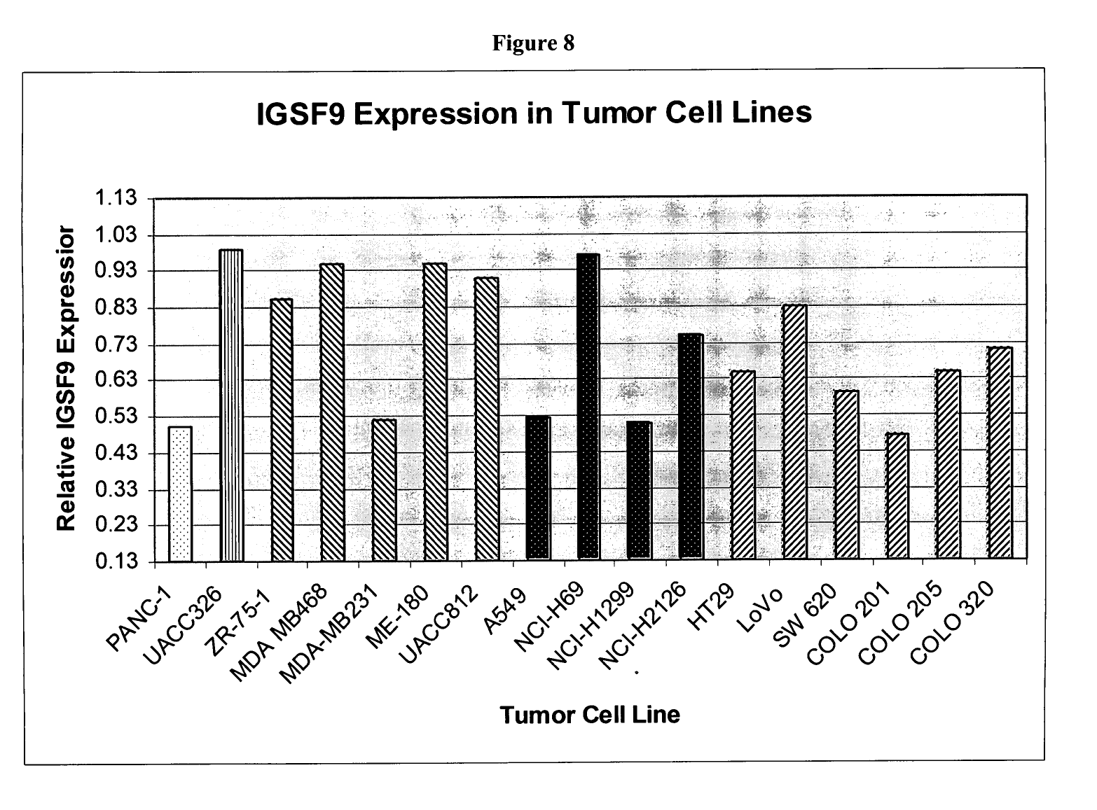

The determined nucleotide sequence of LIV-1 in FIG. 8A contains an open reading frame encoding a protein of about 749 amino acid residues with an initiation codon at position 1 of the nucleotide sequence shown in FIGS. 22A-22B (SEQ ID NOS:28-29), and a predicted leader sequence of about 27 amino acid residues. The amino acid sequence of the predicted LIV-1 protein further contains extracellular domains from about amino acid 28 to about amino acid 317, from about amino acid 373 to about amino acid 417, from about amino acid 674 to about amino acid 678, and from about amino acid 742 to about amino acid 749, as shown in FIG. 22B.

As indicated, nucleic acid molecules of the present invention may be in the form of RNA, such as mRNA, or in the form of DNA, including, for instance, cDNA and genomic DNA obtained by cloning or produced synthetically. The DNA may be double-stranded or single-stranded. Single-stranded DNA or RNA may be the coding strand, also known as the sense strand, or it may be the non-coding strand, also referred to as the antisense strand.

By “isolated” nucleic acid molecule(s) is intended a nucleic acid molecule, DNA or RNA, which has been removed from its native environment For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Isolated nucleic acid molecules of the present invention include DNA molecules comprising an open reading frame (ORF) with an initiation codon at position 1 of the nucleotide sequence shown in FIGS. 1A and 22A (SEQ ID NOS:1 and 28); DNA molecules comprising the coding sequence for the mature IGSF9 and LIV-1 proteins shown in FIGS. 1A and 22A (SEQ ID NOS:1 and 28); DNA molecules comprising the coding sequence shown in FIGS. 9A, 9C, 9E, 9H and 21A (SEQ ID NOS:3, 5, 7 and 12-21); and DNA molecules which comprise a sequence substantially different from those described above but which, due to the degeneracy of the genetic code, still encode the IGSF9 or LIV-1 proteins. Of course, the genetic code is well known in the art. Thus, it would be routine for one skilled in the art to generate the degenerate variants described above.

The present invention is further directed to fragments of the isolated nucleic acid molecules described herein. By a fragment of an isolated nucleic acid molecule having the nucleotide sequence of the nucleotide sequence shown in FIGS. 1A, 9A, 9C, 9E, 9H, 21A, or 22A (SEQ ID NOS:1, 3, 5, 7, 12-21, or 28) is intended fragments at least about 15 nucleotides (nt), and more preferably at least about 20 nt, still more preferably at least about 30 nt, and even more preferably, at least about 40 nt in length which are useful as diagnostic probes and primers as discussed herein. Of course, larger fragments 50-500 nt in length are also useful according to the present invention as are fragments corresponding to most, if not all, of the nucleotide sequence shown in FIGS. 1A, 9A, 9C, 9E, 9H, 21A, or 22A (SEQ ID NOS:1, 3, 5, 7, 12-21, or 28). By a fragment at least 20 nt in length, for example, is intended fragments which include 20 or more contiguous bases from the nucleotide sequence shown in FIGS. 1A, 9A, 9C, 9E, 9H, 21A, or 22A (SEQ ID NOS:1, 3, 5, 7, 12-21, or 28). Preferred nucleic acid fragments of the present invention include nucleic acid molecules encoding epitope-bearing portions of the IGSF9 or LIV-1 proteins. Such isolated molecules, particularly DNA molecules, are useful as probes for gene mapping by in situ hybridization with chromosomes and for detecting expression of the IGSF9 or LIV-1 genes in human tissue, for instance, by Northern blot analysis. As described in detail below, detecting altered IGSF9 or LIV-1 gene expression in certain tissues or bodily fluids is indicative of certain neoplastic disorders.

In another aspect is provided isolated nucleic acid molecules encoding polypeptides of the invention comprising a polynucleotide which hybridizes under stringent hybridization conditions to a portion of the polynucleotide in a nucleic acid molecules of the invention described above. By “stringent hybridization conditions” is intended overnight incubation at 42° C. in a solution comprising: 50% formamide, 5×SSC (750 mM NaCl, 75 mM trisodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C. By a polynucleotide which hybridizes to a “portion” of a polynucleotide is intended a polynucleotide (either DNA or RNA) hybridizing to at least about 15 nt, and more preferably at least about 20 nt, still more preferably at least about 30 nt, and even more preferably about 30-70 nt of the reference polynucleotide. These are useful as diagnostic probes and primers as discussed above and in more detail below.

Of course, polynucleotides hybridizing to a larger portion of the reference polynucleotides, for instance, a portion 50-750 nt in length, or even to the entire length of the reference polynucleotides, are also useful as probes according to the present invention, as are polynucleotides corresponding to most, if not all, of the nucleotide sequence shown in FIGS. 1A, 9A, 9C, 9E, 9H, 21A, or 22A (SEQ ID NOS:1, 3, 5, 7, 12-21, or 28). By a portion of a polynucleotide of “at least 20 nt in length,” for example, is intended 20 or more contiguous nucleotides from the nucleotide sequence of the reference polynucleotide. As indicated, such portions are useful diagnostically either as a probe according to conventional DNA hybridization techniques or as primers for amplification of a target sequence by the polymerase chain reaction (PCR), as described, for instance, in Molecular Cloning, A Laboratory Manual, 2nd. edition, edited by Sambrook, J., Fritsch, E. F. and Maniatis, T., (1989), Cold Spring Harbor Laboratory Press, the entire disclosure of which is hereby incorporated herein by reference.

Since the IGSF9 and LIV-1 nucleotide sequences are provided in FIGS. 1A, 9A, 9C, 9E, 9H, 21A, or 22A (SEQ ID NOS:1, 3, 5, 7, 12-21, or 28), generating polynucleotides which hybridize to a portion of the IGSF9 or LIV-1 molecules would be routine to the skilled artisan. For example, restriction endonuclease cleavage or shearing by sonication of the IGSF9 or LIV-1 molecules could easily be used to generate DNA portions of various sizes which are polynucleotides that hybridize to a portion of the full-length IGSF9 or LIV-1 molecule. Alternatively, the hybridizing polynucleotides of the present invention could be generated synthetically according to known techniques. Of course, a polynucleotide which hybridizes only to a poly A sequence (such as the 3′ terminal poly(A) tract of the IGSF9 or LIV-1 polynucleotides), or to a complementary stretch of T (or U) resides, would not be included in a polynucleotide of the invention used to hybridize to a portion of a nucleic acid of the invention, since such a polynucleotide would hybridize to any nucleic acid molecule containing a poly (A) stretch or the complement thereof.

As indicated, nucleic acid molecules of the present invention which encode the IGSF9 or LIV-1 polypeptides may include, but are not limited to those encoding the amino acid sequence of the mature polypeptide, by itself; the coding sequence for the mature polypeptide and additional sequences, such as those encoding the about 20 amino acid leader or secretory sequence, such as a pre-, or pro- or prepro-protein sequence; the coding sequence of the mature polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing—including splicing and polyadenylation signals, for example—ribosome binding and stability of mRNA; an additional coding sequence which codes for additional amino acids, such as those which provide additional functionalities. Thus, the nucleic acid sequence encoding the polypeptides may be fused to marker sequences, such as a sequence encoding a peptide which facilitates purification of the fused polypeptides. In certain preferred embodiments of this aspect of the invention, the marker amino acid sequence is a hexa-histidine peptide, such as the tag provided in a pQE vector (Qiagen, Inc.), among others, many of which are commercially available. As described in Gentz et al. Proc. Natl. Acad. Sci., USA 86:821-824 (1989) for instance, hexa-histidine provides for convenient purification of the fusion protein. The “HA” tag is another peptide useful for purification which corresponds to an epitope derived from the influenza hemagglutinin protein, which has been described by Wilson et al., Cell 37:767 (1984). Other such fusion proteins include the IGSF9 or LIV-1 polypeptides fused to IgG Fc at the amino- or carboxy-terminus.

The present invention further relates to variants of the nucleic acid molecules of the present invention, which encode portions, analogs or derivatives of the IGSF9 or LIV-1 proteins. Variants may occur naturally, such as a natural allelic variant. By an “allelic variant” is intended one of several alternate forms of a gene occupying a given locus on a chromosome of an organism. Genes II, Lewin, ed. Non-naturally occurring variants may be produced using art-known mutagenesis techniques.

Such variants include those produced by nucleotide substitutions, deletions or additions. The substitutions, deletions or additions may involve one or more nucleotides. The variants may be altered in coding or non-coding regions or both. Alterations in the coding regions may produce conservative or non-conservative amino acid substitutions, deletions or additions. Especially preferred among these are silent substitutions, additions and deletions, which do not alter the properties and activities of the IGSF9 or LIV-1 proteins or portions thereof. Also especially preferred in this regard are conservative substitutions. Most highly preferred are nucleic acid molecules encoding the mature IGSF9 or LIV-1 proteins having the amino acid sequence shown in FIGS. 1A, 9A, 9C, 9E, and 22A (SEQ ID NOS:1, 3, 5, 7, and 28).

Further embodiments of the invention include isolated nucleic acid molecules comprising a polynucleotide having a nucleotide sequence at least 90% identical, and more preferably at least 95%, 96%, 97%, 98% or 99% identical to (a) a nucleotide sequence encoding the IGSF9 or LIV-1 polypeptides having the sequence in FIGS. 1A, 9A, 9C, 9E, 9H, 21A, or 22A (SEQ ID NOS:1, 3, 5, 7, 12-21, or 28); (b) a nucleotide sequence encoding the mature IGSF9 or LIV-1 polypeptide having the amino acid sequence at positions from about 21 to about 718 in FIG. 1B (SEQ ID NO:2), positions from about 28 to about 317 in FIG. 22B (SEQ ID NO:29), positions from about 373 to about 417 in FIG. 22B (SEQ ID NO:29), positions from about 674 to about 678 in FIG. 22B (SEQ ID NO:29), or positions from about 742 to about 749 in FIG. 22B (SEQ ID NO:29); and (c) a nucleotide sequence complementary to any of the nucleotide sequences in (a), or (b) above.

By a polynucleotide having a nucleotide sequence at least, for example, 95% “identical” to a reference nucleotide sequence encoding an IGSF9 or LIV-1 polypeptide is intended that the nucleotide sequence of the polynucleotide is identical to the reference sequence except that the polynucleotide sequence may include up to five point mutations per each 100 nucleotides of the reference nucleotide sequence encoding either the IGSF9 or LIV-1 polypeptides. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 90%, 95%, 96%, 97%, 98% or 99% identical to, for instance, the nucleotide sequence shown in FIGS. 1A, 9A, 9C, 9E, 9H, 21A, or 22A (SEQ ID NOS:1, 3, 5, 7, 12-21, or 28), can be determined conventionally using known computer programs such as the Bestfit program (Wisconsin Sequence Analysis Package, Version 8 for Unix, Genetics Computer Group, University Research Park, 575 Science Drive, Madison, Wis. 53711. Bestfit uses the local homology algorithm of Smith and Waterman (Advances in Applied Mathematics 2: 482-489, 1981) to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

Of course, due to the degeneracy of the genetic code, one of ordinary skill in the art will immediately recognize that a large number of the nucleic acid molecules having a sequence at least 90%, 95%, 96%, 97%, 98%, or 99% identical to the nucleic acid sequences shown in FIGS. 1A, 9A, 9C, 9E, 9H, 21A, or 22A (SEQ ID NOS:1, 3, 5, 7, 12-21, or 28), will encode a polypeptide having IGSF9 or LIV-1 protein activity. In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having either IGSF9 or LIV-1 protein activity. This is because the skilled artisan is fully aware of amino acid substitutions that are either less likely or not likely to significantly effect protein function (e.g., replacing one aliphatic amino acid with a second aliphatic amino acid).

For example, guidance concerning how to make phenotypically silent amino acid substitutions is provided in Bowie, J. U., et al, “Deciphering the Message in Protein Sequences: Tolerance to Amino Acid Substitutions,” Science 247:1306-1310 (1990), wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selections or screens to identify sequences that maintain functionality. As the authors state, these studies have revealed that proteins are surprisingly tolerant of amino acid substitutions. The authors further indicate which amino acid changes are likely to be permissive at a certain position of the protein. For example, most buried amino acid residues require nonpolar side chains, whereas few features of surface side chains are generally conserved. Other such phenotypically silent substitutions are described in Bowie, J. U., et al., supra, and the references cited therein.

Cancer Diagnosis and Therapy

Polypeptides of the invention may be involved in cancer cell generation, proliferation or metastasis. Detection of the presence or amount of the polynucleotides or polypeptides of the invention may be useful for the diagnosis and/or prognosis of one or more types of cancer. For example, the presence or increased expression of a polynucleotide/polypeptide of the invention may indicate a hereditary risk of cancer, a precancerous condition, or an ongoing malignancy. Conversely, a defect in the gene or absence of the polypeptide may be associated with a cancer condition. Identification of single nucleotide polymorphisms associated with cancer or a predisposition to cancer may also be useful for diagnosis or prognosis.

Cancer treatments promote tumor regression by inhibiting tumor cell proliferation, inhibiting angiogensis (growth of new blood vessels that is necessary to support tumor growth) and/or prohibiting metastasis by reducing tumor cell motility or invasiveness. Therapeutic compositions of the invention may be effective in adult and pediatric oncology including in solid phase tumors/malignancies, lung cancers including small cell carcinoma and non-small cell cancers, breast cancers including small cell carcinoma and ductal carcinoma, gastrointestinal cancers including esophageal cancer, stomach cancer, colon cancer, colorectal cancer and polyps associated with colorectal neoplasia, urologic cancers including bladder cancer and prostate cancer, and malignancies of the female genital tract including ovarian carcinoma, uterine (including endometrial) cancers, and solid tumor in the ovarian follicle.

Polypeptides, polynucleotides, antibodies (or antigen binding fragments thereof) or modulators of polypeptides of the invention (including inhibitors and stimulators of the biological activity of the polypeptide of the invention) may be administered to treat cancer. Therapeutic compositions can be administered in therapeutically effective dosages alone or in combination with adjuvant cancer therapy such as surgery, chemotherapy, radiotherapy, thermotherapy, and laser therapy, and may provide a beneficial effect, e.g. reducing tumor size, slowing rate of tumor growth, inhibiting metastasis, or otherwise improving overall clinical condition, without necessarily eradicating the cancer.

The composition can also be administered in therapeutically effective amounts as a portion of an anti-cancer cocktail. An anti-cancer cocktail is a mixture of the polypeptide or modulator of the invention with one or more anti-cancer drugs in addition to a pharmaceutically acceptable carrier for delivery. The use of anti-cancer cocktails as cancer treatment is routine. Anti-cancer drugs that are well known in the art and can be used as a treatment in combination with polypeptide or modulator of the invention include: Actinomycin D, Aminoglutethimide, Asparaginase, Bleomycin, Busulfan, Carboplatin, Carmustine, Chalorambucil, Cisplatin (cis-DDP), Cyclophosphamide, Cytarabine HCl (Cytosine arabinoside), Dacarbazine, Dactinomycin, Daunorubicin HCl, Doxorubicin HCl, Estramustine phosphate sodium, Etoposide (V16-213), Floxuridine, 5-Fluorouracil (5-Fu), Flutamide, Hydroxyurea (hydroxycarbamide), Ifosfamide, Interferon Alpha-2a, Interferon Alpha-2b, Leuprolide acetate (LHRH-releasing factor analog), Lomustine, Mechlorethamine HCl (nitrogen mustard), Melphalan, Mercaptopurine, Mesna, Methotrexate (MTX), Mitomycin, Mitoxantrone HCl, Octreotide, Plicamycin, Procarbazine HCl, streptozocin, Tamoxifen citrate, Thioguanine, Thiotepa, Vinblastine sulfate, Vincristine sulfate, Amsacrine, Azacitidine, Hexamethylmelamine, Interleukin-2, Mitoguazone, Pentostatin, Semustine, Teniposide, and Vindesine sulfate.

In addition, therapeutic compositions of the invention may be used for prophylactic treatment of cancer. There are hereditary conditions and/or environmental situations (e.g. exposure to carcinogens) known in the art that predispose an individual to developing cancers. Under these circumstances, it may be beneficial to treat these individuals with therapeutically effective doses of the polypeptide of the invention to reduce the risk of developing cancers.

In vitro models can be used to determine the effective doses of the polypeptide of the invention as a potential cancer treatment. These in vitro models include proliferation assays of cultured tumor cells, growth of cultured tumor cells in soft agar (see Freshney, (1987) Culture of Animal Cells: A Manual of Basic Technique, Wily-Liss, New York, N.Y. Ch18 and Ch21), tumor systems in nude mice as described in Giovanella et al., J. Natl. Can Inst. 52:921-30 (1974), mobility and invasive potential of tumor cells in Boyden Chamber assays as described in Pilkington et al., Anticancer Res. 17:4107-9 (1997), and angiogensis assays such as induction of vascularization of the chick chorioallantoic membrane or induction of vascular endothelial cell migration as described in Ribatta et al., Intl. J. Dev. Biol. 40:1189-97 (1999) and Li et al., Clin. Exp. Metastasis 17:423-9 (1999), respectively. Suitable tumor cells lines are available, e.g. from American Type Tissue Culture Collection catalogs.

However, as discussed above, selected embodiments of the invention comprise the administration of modified antibodies to cancer patients or in combination or conjunction with one or more adjunct therapies such as radiotherapy or chemotherapy (i.e. a combined therapeutic regimen). As used herein, the administration of modified antibodies in conjunction or combination with an adjunct therapy means the sequential, simultaneous, coextensive, concurrent, concomitant or contemporaneous administration or application of the therapy and the disclosed antibodies. Those skilled in the art will appreciate that the administration or application of the various components of the combined therapeutic regimen may be timed to enhance the overall effectiveness of the treatment. For example, chemotherapeutic agents could be administered in standard, well known courses of treatment followed within a few weeks by radioimmunoconjugates of the present invention. Conversely, cytotoxin associated modified antibodies could be administered intravenously followed by tumor localized external beam radiation. In yet other embodiments, the modified antibody may be administered concurrently with one or more selected chemotherapeutic agents in a single office visit. A skilled artisan (e.g. an experienced oncologist) would be readily be able to discern effective combined therapeutic regimens without undue experimentation based on the selected adjunct therapy and the teachings of this specification.

In this regard it will be appreciated that the combination of the modified antibody (with or without cytotoxin) and the chemotherapeutic agent may be administered in any order and within any time frame that provides a therapeutic benefit to the patient. That is, the chemotherapeutic agent and modified antibody may be administered in any order or concurrently. In selected embodiments the modified antibodies of the present invention will be administered to patients that have previously undergone chemotherapy. In yet other embodiments, the modified antibodies and the chemotherapeutic treatment will be administered substantially simultaneously or concurrently. For example, the patient may be given the modified antibody while undergoing a course of chemotherapy. In preferred embodiments the modified antibody will be administered within 1 year of any chemotherapeutic agent or treatment. In other preferred embodiments the modified antibody will be administered within 10, 8, 6, 4, or 2 months of any chemotherapeutic agent or treatment. In still other preferred embodiments the modified antibody will be administered within 4, 3, 2 or 1 week of any chemotherapeutic agent or treatment. In yet other embodiments the modified antibody will be administered within 5, 4, 3, 2 or 1 days of the selected chemotherapeutic agent or treatment. It will further be appreciated that the two agents or treatments may be administered to the patient within a matter of hours or minutes (i.e. substantially simultaneously).

In this regard it will further be appreciated that the modified antibodies of this invention may be used in conjunction or combination with any chemotherapeutic agent or agents or regimen (e.g. to provide a combined therapeutic regimen) that eliminates, reduces, inhibits or controls the growth of neoplastic cells in vivo. As discussed, such agents often result in the reduction of red marrow reserves. This reduction may be offset, in whole or in part, by the diminished myelotoxicity of the compounds of the present invention that advantageously allow for the aggressive treatment of neoplasms in such patients. In other preferred embodiments the radiolabeled immunoconjugates disclosed herein may be effectively used with radiosensitizers that increase the susceptibility of the neoplastic cells to radionuclides. For example, radiosensitizing compounds may be administered after the radiolabeled modified antibody has been largely cleared from the bloodstream but still remains at therapeutically effective levels at the site of the tumor or tumors.

With respect to these aspects of the invention, exemplary chemotherapeutic agents that are compatible with this invention include alkylating agents, vinca alkaloids (e.g., vincristine and vinblastine), procarbazine, methotrexate and prednisone. The four-drug combination MOPP (mechlethamine (nitrogen mustard), vincristine (Oncovin), procarbazine and prednisone) is very effective in treating various types of lymphoma and comprises a preferred embodiment of the present invention. In MOPP-resistant patients, ABVD (e.g., adriamycin, bleomycin, vinblastine and dacarbazine), ChlVPP (chlorambucil, vinblastine, procarbazine and prednisone), CABS (lomustine, doxorubicin, bleomycin and streptozotocin), MOPP plus ABVD, MOPP plus ABV (doxorubicin, bleomycin and vinblastine) or BCVPP (carmustine, cyclophosphamide, vinblastine, procarbazine and prednisone) combinations can be used. Arnold S. Freedman and Lee M. Nadler, Malignant Lymphomas, in HARRISON'S PRINCIPLES OF INTERNAL MEDICINE 1774-1788 (Kurt J. Isselbacher et al., eds., 13^{th} ed. 1994) and V. T. DeVita et al., (1997) and the references cited therein for standard dosing and scheduling. These therapies can be used unchanged, or altered as needed for a particular patient, in combination with one or more modified antibodies as described herein.

Additional regimens that are useful in the context of the present invention include use of single alkylating agents such as cyclophosphamide or chlorambucil, or combinations such as CVP (cyclophosphamide, vincristine and prednisone), CHOP (CVP and doxorubicin), C-MOPP (cyclophosphamide, vincristine, prednisone and procarbazine), CAP-BOP (CHOP plus procarbazine and bleomycin), m-BACOD (CHOP plus methotrexate, bleomycin and leucovorin), ProMACE-MOPP (prednisone, methotrexate, doxorubicin, cyclophosphamide, etoposide and leucovorin plus standard MOPP), ProMACE-CytaBOM (prednisone, doxorubicin, cyclophosphamide, etoposide, cytarabine, bleomycin, vincristine, methotrexate and leucovorin) and MACOP-B (methotrexate, doxorubicin, cyclophosphamide, vincristine, fixed dose prednisone, bleomycin and leucovorin). Those skilled in the art will readily be able to determine standard dosages and scheduling for each of these regimens. CHOP has also been combined with bleomycin, methotrexate, procarbazine, nitrogen mustard, cytosine arabinoside and etoposide. Other compatible chemotherapeutic agents include, but are not limited to, 2-chlorodeoxyadenosine (2-CDA), 2′-deoxycoformycin and fludarabine.

The amount of chemotherapeutic agent to be used in combination with the modified antibodies of this invention may vary by subject or may be administered according to what is known in the art. See for example, Bruce A Chabner et al., Antineoplastic Agents, in GOODMAN & GILMAN'S THE PHARMACOLOGICAL BASIS OF THERAPEUTICS 1233-1287 ((Joel G. Hardman et al., eds., 9^{th} ed. 1996).

As previously discussed, the modified antibodies of the present invention, immunoreactive fragments or recombinants thereof may be administered in a pharmaceutically effective amount for the in vivo treatment of mammalian malignancies. In this regard, it will be appreciated that the disclosed antibodies will be formulated so as to facilitate administration and promote stability of the active agent. Preferably, pharmaceutical compositions in accordance with the present invention comprise a pharmaceutically acceptable, non-toxic, sterile carrier such as physiological saline, non-toxic buffers, preservatives and the like. For the purposes of this application, a pharmaceutically effective amount of the modified antibody, immunoreactive fragment or recombinant thereof, conjugated or unconjugated to a therapeutic agent, shall be held to mean an amount sufficient to achieve effective binding with selected immunoreactive antigens on neoplastic cells and provide for an increase in the death of those cells. Of course, the pharmaceutical compositions of the present invention may be administered in single or multiple doses to provide for a pharmaceutically effective amount of the modified antibody.

More specifically, the disclosed antibodies and methods should be useful for reducing tumor size, inhibiting tumor growth and/or prolonging the survival time of tumor-bearing animals. Accordingly, this invention also relates to a method of treating tumors in a human or other animal by administering to such human or animal an effective, non-toxic amount of modified antibody. One skilled in the art would be able, by routine experimentation, to determine what an effective, non-toxic amount of modified antibody would be for the purpose of treating malignancies. For example, a therapeutically active amount of a modified antibody may vary according to factors such as the disease stage (e.g., stage I versus stage IV), age, sex, medical complications (e g., immunosuppressed conditions or diseases) and weight of the subject, and the ability of the antibody to elicit a desired response in the subject. The dosage regimen may be adjusted to provide the optimum therapeutic response. For instance, several divided doses may be administered daily, or the dose may be proportionally reduced as indicated by the exigencies of the therapeutic situation. Generally, however, an effective dosage is expected to be in the range of about 0.05 to 100 milligrams per kilogram body weight per day and more preferably from about 0.5 to 10, milligrams per kilogram body weight per day.

In keeping with the scope of the present disclosure, the modified antibodies of the invention may be administered to a human or other animal in accordance with the aforementioned methods of treatment in an amount sufficient to produce such effect to a therapeutic or prophylactic degree. The antibodies of the invention can be administered to such human or other animal in a conventional dosage form prepared by combining the antibody of the invention with a conventional pharmaceutically acceptable carrier or diluent according to known techniques. It will be recognized by one of skill in the art that the form and character of the pharmaceutically acceptable carrier or diluent is dictated by the amount of active ingredient with which it is to be combined, the route of administration and other well-known variables. Those skilled in the art will further appreciate that a cocktail comprising one or more species of monoclonal antibodies according to the present invention may prove to be particularly effective.

Methods of preparing and administering conjugates of the antibody, immunoreactive fragments or recombinants thereof, and a therapeutic agent are well known to or readily determined by those skilled in the art. The route of administration of the antibodies (or fragment thereof) of the invention may be oral, parenteral, by inhalation or topical. The term parenteral as used herein includes intravenous, intraarterial, intraperitoneal, intramuscular, subcutaneous, rectal or vaginal administration. The intravenous, intraarterial, subcutaneous and intramuscular forms of parenteral administration are generally preferred. While all these forms of administration are clearly contemplated as being within the scope of the invention, a preferred administration form would be a solution for injection, in particular for intravenous or intraarterial injection or drip. Usually, a suitable pharmaceutical composition for injection may comprise a buffer (e.g. acetate, phosphate or citrate buffer), a surfactant (e.g. polysorbate), optionally a stabilizer agent (e.g. human albumin), etc. However, in other methods compatible with the teachings herein, the modified antibodies can be delivered directly to the site of the malignancy site thereby increasing the exposure of the neoplastic tissue to the therapeutic agent.

Preparations for parenteral administration includes sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. In the subject invention, pharmaceutically acceptable carriers include, but are not limited to, 0.01-0.1M and preferably 0.05M phosphate buffer or 0.8% saline. Other common parenteral vehicles include sodium phosphate solutions, Ringer's dextrose, dextrose and sodium chloride, lactated Ringer's, or fixed oils. Intravenous vehicles include fluid and nutrient replenishers, electrolyte replenishers, such as those based on Ringer's dextrose, and the like. Preservatives and other additives may also be present such as for example, antimicrobials, antioxidants, chelating agents, and inert gases and the like.

More particularly, pharmaceutical compositions suitable for injectable use include sterile aqueous solutions (where water soluble) or dispersions and sterile powders for the extemporaneous preparation of sterile injectable solutions or dispersions. In such cases, the composition must be sterile and should be fluid to the extent that easy syringability exists. It should be stable under the conditions of manufacture and storage and will preferably be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants.

Prevention of the action of microorganisms can be achieved by various antibacterial and antifungal agents, for example, parabens, chlorobutanol, phenol, ascorbic acid, thimerosal and the like. In many cases, it will be preferable to include isotonic agents, for example, sugars, polyalcohols, such as mannitol, sorbitol, or sodium chloride in the composition. Prolonged absorption of the injectable compositions can be brought about by including in the composition an agent which delays absorption, for example, aluminum monostearate and gelatin.

In any case, sterile injectable solutions can be prepared by incorporating an active compound (e.g., a modified antibody by itself or in combination with other active agents) in the required amount in an appropriate solvent with one or a combination of ingredients enumerated herein, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yields a powder of an active ingredient plus any additional desired ingredient from a previously sterile-filtered solution thereof. The preparations for injections are processed, filled into containers such as ampoules, bags, bottles, syringes or vials, and sealed under aseptic conditions according to methods known in the art. Further, the preparations may be packaged and sold in the form of a kit such as those described in co-pending U.S. Ser. No. 09/259,337 and U.S. Ser. No. 09/259,338 each of which is incorporated herein by reference. Such articles of manufacture will preferably have labels or package inserts indicating that the associated compositions are useful for treating a subject suffering from, or predisposed to, cancer, malignancy or neoplastic disorders.

As discussed in detail above, the present invention provides compounds, compositions, kits and methods for the treatment of neoplastic disorders in a mammalian subject in need of treatment thereof. Preferably, the subject is a human. The neoplastic disorder (e.g., cancers and malignancies) may comprise solid tumors such as melanomas, gliomas, sarcomas, and carcinomas as well as myeloid or hematologic malignancies such as lymphomas and leukemias. In general, the disclosed invention may be used to prophylactically or therapeutically treat any neoplasm containing IGSF9 or LIV-1 as antigenic markers that allows for the targeting of the cancerous cells by the modified antibody. Exemplary cancers that may be treated include, but are not limited to, prostate, colon, breast, ovarian and lung. In addition to the aforementioned neoplastic disorders, it will be appreciated that the disclosed invention may advantageously be used to treat additional malignancies bearing IGSF9 or LIV-1.

Receptor/Ligand Activity

Polypeptides of the present invention may also demonstrate activity as receptors, receptor ligands or inhibitors or agonists of receptor/ligand interactions. A polynucleotide of the invention can encode a polypeptide exhibiting such characteristics. Examples of such receptors and ligands include, without limitation, cytokine receptors and their ligands, receptor kinases and their ligands (including without limitation, cellular adhesion molecules (such as selectins, intergrins and their ligands) and receptor/ligand pairs involved in antigen presentation, antigen recognition and development of cellular and humoral immune responses. Receptors and ligands are also useful for screening of potential peptide or small molecule inhibitors of the relevant receptor/ligand interactions. A polypeptide of the present invention (including, without limitation, fragments of receptors and ligands) may itself be useful as inhibitors of receptor/ligand interactions.

Suitable assays for determining receptor-ligand activity of the polypeptides of the invention include without limitation those described in: Current Protocols in Immunology, Ed by J. E. Coligan, et al., Pub. Greene Publishing Associates and Wiley-Interscience (Chapter 7.28, Measurement of Cellular Adhesion under static conditions 7.28.1-7.28.22), Takai et al., Proc. Natl. Acad. Sci. USA 84:6864-6868, 1987; Bierer et al., J. Exp. Med. 168:1145-1156, 1988; Rosenstein et al, J. Exp. Med. 169:149-160 1989; Stoltenborg et al., J. Immunol. Methods 175:56-98, 1994; Stitt et al., Cell 80:661-670, 1995.

By way of example, the polypeptides of the invention may be used as a receptor for a ligand(s) thereby transmitting the biological activity of that ligand(s). Ligands may be identified through binding assays, affinity chromatography, dihybrid screening assays, BIAcore assays, gel overlay assays, or other methods known in the art.

Studies characterizing drugs or proteins as agonist or antagonist or partial agonists or a partial antagonist requires the use of the other proteins as competing ligands. The polypeptides of the present invention or ligand(s) thereof may be labeled by being coupled to radioisotopes, calorimetric molecules or a toxin molecules by conventional methods. (“Guide to Protein Purification” Murray P. Deutscher (ed) Methods in Enzymology Vol. 182 (1990) Academic Press, Inc. San Diego). Examples of radioisotopes include, but are not limited to, tritium and carbon-14. Examples of calorimetric molecules include, but are not limited to, fluorescent molecules such as fluorescamine, or rhodamine or other calorimetric molecules. Examples of toxins include, but are not limited to ricin.

Assays for Receptor Activity

The invention also provides methods to detect specific binding of polypeptides of the invention, e.g. a ligand or a receptor. The art provides numerous assays particularly useful for identifying previously unknown binding partners for receptor polypeptides of the invention. For example, expression cloning using mammalian or bacterial cells, or dihybrid screening assays can be used to identify polynucleotides encoding binding partners. As another example, affinity chromatography with the appropriate immobilized polypeptide of the invention can be used to isolate polypeptides that recognize and bind polypeptides of the invention. There are a number of different libraries used for the identification of compounds, and in particular small molecules, that modulate (i.e., increase or decrease) biological activity of a polypeptide of the invention. Ligands for receptor polypeptides of the invention can also be identified by adding exogenous ligands, or cocktails of ligands to two cells populations that are genetically identical except for the expression of the receptor of the invention: one cell population expresses the receptor of the invention whereas the other does not. The response of the two cell populations to the addition of ligand(s) are then compared. Alternatively, an expression library can be co-expressed with the polypeptide of the invention in cells and assayed for an autocrine response to identify potential ligand(s). As still another example, BIAcore assays, gel overlay assays, or other methods known in the art can be used to identify binding partner polypeptides, including, (1) organic and inorganic chemical libraries, (2) natural product libraries, and (3) combinatorial libraries comprised of random peptides, oligonucleotides or organic molecules.

The role of downstream intracellular signaling molecules in the signaling cascade of the polypeptides of the invention can be determined. For example, a chimeric protein in which the cytoplasmic domain of the polypeptide of the invention is fused to the extracellular portion of a protein, whose ligand has been identified, is produced in a host cell. The cell is then incubated with the ligand specific for the extracellular portion of the chimeric protein, thereby activation the chimeric receptor. Known downstream proteins involved in intracellular signaling can then be assayed for expected modifications i.e. phosphorylation. Other methods known to those in the are can also be used to identify signaling molecules involved in receptor activity.

Antisense Oligonucleotides

Another aspect of the invention pertains to isolated antisense nucleic acid molecules that are hybridizable to or complementary to the nucleic acid molecules comprising the nucleotide sequences of FIGS. 1A, 9A, 9C, 9E, 9H, 21A, or 22A (SEQ ID NOS:1, 3, 5, 7, 12-21, or 28), or fragments, analogs or derivatives thereof. An antisense nucleic acid comprises a nucleotide sequence that is complementary to a sense nucleic acid encoding a protein. In specific aspects, antisense nucleic acid molecules are provided that comprise a sequence complementary to at least about 10, 25, 50, 100, 250 or 500 nucleotides or an entire coding strand, or to only a portion thereof. Nucleic acid molecules encoding fragments, homologs, derivatives and analogs of a protein of FIGS. 1B, 9B, 9D, 9F, 21B, or 22B (SEQ ID NOS:2, 4, 6, 8, 22-27, or 29), or antisense nucleic acids complementary to a nucleic acid sequence of FIGS. 1A, 9A, 9C, 9E, 9H, 21A, or 22A (SEQ ID NOS:1, 3, 5, 7, 12-21, or 28), are additionally provided.

In one embodiment, an antisense nucleic acid molecule is antisense to a coding region of the coding strand of a nucleotide sequence of the invention. The term coding region refers to the region of the nucleotide sequence comprising codons which are translated into amino acid residues. In another embodiment, the antisense nucleic acid molecule is antisense to a noncoding region of the coding strand of a nucleotide sequence of the invention. The term noncoding region refers 5′ and 3′ sequences which flank the coding region that are not translated into amino acids (i.e. also referred to as 5′ and 3′ untranslated regions).

As used in this disclosure the term antisense nucleic acid encompasses both oligomeric nucleic acid moieties of the type found in nature, such as the deoxyribonucleotide and ribonucleotide structures of DNA and RNA, and man-made analogs which are capable of binding to nucleic acids found in nature. The oligonucleotides of the present invention can be based upon ribonucleotide or deoxyribonucleotide monomers linked by phosphodiester bonds, or by analogues linked by methyl phosphonate, phosphorothioate, or other bonds. They may also comprise monomer moieties which have altered base structures or other modifications, but which still retain the ability to bind to naturally occurring DNA and RNA structures.

Given the coding strand sequences encoding the nucleic acids disclosed herein (e.g. SEQ ID NOS:1, 3, 5, 7, 12-21, or 28), antisense nucleic acids of the invention can be designed according to the rules of Watson and Crick or Hoogsteen base pairing. The antisense molecule can be complementary to the entire coding region of an mRNA, but more preferably is an oligonucleotide that is antisense to only a portion of the coding or noncoding region surrounding the translation start site of a mRNA. An antisense oligonucleotide can be, for example, about 5, 10, 15, 20, 25, 30, 35, 40, 45 or 50 nucleotides in length. To select the preferred length for an antisense oligonucleotide, a balance must be struck to gain the most favorable characteristics. Shorter oligonucleotides 10-15 bases in length readily enter cells, but have lower gene specificity. In contrast, longer oligonucleotides of 20-30 bases offer superior gene specificity, but show decreased kinetics of uptake into cells. See Stein et al., PHOSPHOROTHIOATE OLIGODEOXYNUCLEOTIDE ANALOGUES in “Oligodeoxynucleotides—Antisense Inhibitors of Gene Expression” Cohen, Ed. McMillan Press, London (1988).

An antisense nucleic acid of the invention can be constructed using chemical synthesis or enzymatic ligation reactions using procedures known in the art. For example, an antisense nucleic acid can be chemically synthesized using naturally occurring nucleotides or variously modified nucleotides designed to increase the biological stability of the molecules or to increase the physical stability of the duplex formed between the antisense and sense nucleic acids (e.g. phosphorothioate derivative and acridine substituted nucleotides can be used.

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactoslqueosine, inosine, N6-isopentanyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a protein according to the invention to thereby inhibit expression of the protein, e.g., by inhibiting transcription and/or translation. The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules can be modified to target selected cells and then administered systemically. For Example, for systemic administration, antisense molecules can be modified such that they specifically bind to receptors or antigens expressed on a selected cell surface, e.g., by linking the antisense nucleic acid molecules to peptides or antibodies that bind to cell surface receptors or antigens. The antisense nucleic acid molecules can also be delivered to cells using the vectors described herein. To achieve sufficient intracellular concentrations of antisense molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other (Gaultier et al., Nucleic Acids Res 15:6625-6641 (1987)). The antisense nucleic acid molecule can also comprise a 2′-o-methlribonucleotide (Inoue et al., Nucleic Acids Res 15:6131-6148 (1987)) or a chimeric RNA-DNA analogue (Inoue et al., FEBS Lett 215:327-330 (1987)).

Tumor Vaccine

The peptides of the present invention, or analogs thereof, may be used to treat or prevent a neoplastic disorder in the form of a vaccine composition. The peptides of the present invention or analogs thereof which have immune-stimulating activity may be modified to provide desired attributes other than improved serum half life. For instance, the ability of the peptides to induce CTL activity can be enhanced by linkage to a sequence which contains at least one epitope that is capable of inducing a T helper cell response. Particularly preferred immunogenic peptides/T helper conjugates are linked by a spacer molecule. The spacer is typically comprised of relatively small, neutral molecules, such as amino acids or amino acid mimetics, which are substantially uncharged under physiological conditions and may have linear or branched side chains. The spacers are typically selected from, e.g., Ala, Gly, or other neutral spacers of nonpolar amino acids or neutral polar amino acids. It will be understood that the optionally present spacer need not be comprised of the same residues and thus may be a hetero- or homo-oligomer. When present, the spacer will usually be at least one or two residues, more usually three to six residues. Alternatively, the CTL peptide may be linked to the T helper peptide without a spacer.

The immunogenic peptide may be linked to the T helper peptide either directly or via a spacer either at the amino or carboxy terminus of the CTL peptide. The amino terminus of either the immunogenic peptide or the T helper peptide may acylated. Exemplary T helper peptides include tetanus toxoid 830-843, influenza 307-319, malaria circumsporozoite 382-398 and 378-389.

In some embodiments it may be desirable to include in the vaccine compositions of the invention at least one component which is immunostimulatory. Therefore, the invention also includes the use of a non-nucleic acid adjuvant in some aspects. The non-nucleic acid adjuvant in some embodiments is an adjuvant that creates a depo effect, an immune stimulating adjuvant, or an adjuvant that creates a depo effect and stimulates the immune system. Preferably the adjuvant that creates a depo effect is selected from the group consisting of alum (e.g., aluminum hydroxide, aluminum phosphate) emulsion based formulations including mineral oil, non-mineral oil, water-in-oil or oil-in-water emulsions, such as the Seppic ISA series of Montanide adjuvants; MF-59; and PROVAX™. In a more preferred embodiment, the immunostimulatory agent is PROVAX™.

In some embodiments the immune stimulating adjuvant is selected from the group consisting of saponins purified from the bark of the Q. saponaria tree, such as QS21; poly[di(carboxylatophenoxy)phosphazene (PCPP) derivatives of lipopolysaccharides such as monophosphorlyl lipid (MPL), muramyl dipeptide (MDP) and threonyl muramyl dipeptide (tMDP); OM-174; and Leishmania elongation factor. In one embodiment the adjuvant that creates a depo effect and stimulates the immune system is selected from the group consisting of ISCOMS; SB-AS2; SB-AS4; non-ionic block copolymers that form micelles such as CRL 1005; and Syntex Adjuvant Formulation.

The immunogenic peptides can be prepared synthetically, or by recombinant DNA technology or isolated from natural sources such as whole viruses or tumors. Although the peptide will preferably be substantially free of other naturally occurring host cell proteins and fragments thereof, in some embodiments the peptides can be synthetically conjugated to native fragments or particles. The polypeptides or peptides can be a variety of lengths, either in their neutral (uncharged) forms or in forms which are salts, and either free of modifications such as glycosylation, side chain oxidation, or phosphorylation or containing these modifications, subject to the condition that the modification not destroy the biological activity of the polypeptides as herein described.

Alternatively, recombinant DNA technology may be employed wherein a nucleotide sequence which encodes an immunogenic peptide of interest is inserted into an expression vector, transformed or transfected into an appropriate host cell and cultivated under conditions suitable for expression. These procedures are generally known in the art, as described generally in Sambrook et al., Molecular Cloning, A Laboratory Manual, Cold Spring Harbor Press, Cold Spring Harbor, N.Y. (1982), which is incorporated herein by reference. Thus, fusion proteins which comprise one or more peptide sequences of the invention can be used to present the appropriate T cell epitope.

The peptides of the present invention and pharmaceutical and vaccine compositions thereof are useful for administration to mammals, particularly humans, to treat neoplasms. Examples of neoplastic diseases which can be treated using the immunogenic peptides of the invention include lung, ovarian, breast and prostate cancer.

Vaccine compositions containing the peptides of the invention are administered to a patient susceptible to or otherwise at risk of viral infection or cancer to elicit an immune response against the antigen and thus enhance the patient's own immune response capabilities. Such an amount is defined to be an “immunogenically effective dose.” In this use, the precise amounts again depend on the patient's state of health and weight, the mode of administration, the nature of the formulation, etc., but generally range from about 1.0 μg to about 5000 μg per 70 kilogram patient, more commonly from about 10 μg to about 500 μg mg per 70 kg of body weight.

For therapeutic or immunization purposes, the peptides of the invention can also be expressed by attenuated viral hosts, such as vaccinia or fowlpox. This approach involves the use of vaccinia virus as a vector to express nucleotide sequences that encode the peptides of the invention. Upon introduction into an acutely or chronically infected host or into a non-infected host, the recombinant vaccinia virus expresses the immunogenic peptide, and thereby elicits a host CTL response. Vaccinia vectors and methods useful in immunization protocols are described in, e.g., U.S. Pat. No. 4,722,848, incorporated herein by reference. Another vector is BCG (Bacille Calmette Guerin). BCG vectors are described in (Stover et al., Nature 351:456-460 (1991)) which is incorporated herein by reference. A wide variety of other vectors useful for therapeutic administration or immunization of the peptides of the invention, e.g., Salmonella typhi vectors and the like, will be apparent to those skilled in the art from the description herein.

Antigenic peptides may be used to elicit CTL ex vivo, as well. The resulting CTL, can be used to treat chronic infections (viral or bacterial) or tumors in patients that do not respond to other conventional forms of therapy, or will not respond to a peptide vaccine approach of therapy. Ex vivo CTL responses to a particular pathogen (infectious agent or tumor antigen) are induced by incubating in tissue culture the patient's CTL precursor cells (CTLp) together with a source of antigen-presenting cells (APC) and the appropriate immunogenic peptide. After an appropriate incubation time (typically 1-4 weeks), in which the CTLp are activated and mature and expand into effector CTL, the cells are infused back into the patient, where they will destroy their specific target cell (an infected cell or a tumor cell).

The peptides may also find use as diagnostic reagents. For example, a peptide of the invention may be used to determine the susceptibility of a particular individual to a treatment regimen which employs the peptide or related peptides, and thus may be helpful in modifying an existing treatment protocol or in determining a prognosis for an affected individual. In addition, the peptides may also be used to predict which individuals will be at substantial risk for developing chronic infection.

Anti-Idiotypic Antibodies

The present invention is also directed to methods which utilize anti-idiotypic antibodies for tumor immunotherapy and immunoprophylaxis. The invention relates to the manipulation of the idiotypic network of the immune system for therapeutic advantage. Immunization with anti-idiotypic antibodies (Ab2) can induce the formation of anti-anti-idiotypic immunoglobulins, some of which have the same antigen specificity as the antibody (Ab1) used to derive the anti-idiotype. This creates a powerful paradigm for manipulation of immune responses by offering a mechanism for generating and amplifying antigen-specific recognition in the immune system. An immune response to tumors appears to involve idiotype-specific recognition of tumor antigen; the present invention relates to strategies for manipulating this recognition towards achieving therapeutic benefit. Particular embodiments of the invention include the use of anti-idiotypic antibody for immunization against tumor, for activation of lymphocytes used in adoptive immunotherapy, and for inhibition of immune suppression mediated by suppressor T cells or suppressor factors expressing an idiotope directed against a tumor antigen. The anti-idiotypic antibodies, or fragments thereof, can also be used to monitor anti-antibody induction in patients undergoing passive immunization to a tumor antigen by administration of anti-tumor antibody.

In a specific embodiment, the induction of anti-idiotypic antibodies in vivo, by administration of anti-tumor antibody or immune cells or factors exhibiting the anti-tumor idiotope, can be of therapeutic value.

The present invention is also directed to anti-idiotypic MAb molecules, or fragments of the anti-idiotypic MAb molecules, or modifications thereof, that recognize an idiotype that is directed against IGSF9 or LIV-1.

The MAb molecules of the present invention include whole monoclonal antibody molecules and fragments or any chemical modifications of these molecules, which contain the antigen combining site that binds to the idiotype of another antibody molecule(s) with specificity to IGSF9 or LIV-1. Monoclonal antibody fragments containing the idiotype of the MAb molecule could be generated by various techniques. These include, but are not limited to: the F(ab′)₂ fragment which can be generated by treating the antibody molecule with pepsin, the Fab′ fragments which can be generated by reducing the disulfide bridges of the F(ab′)₂ fragment, and the 2Fab or Fab fragments which can be generated by treating the antibody molecule with papain and a reducing agent to reduce the disulfide bridges.

Depending upon its intended use, the anti-idiotype antibodies of the invention may be chemically modified by the attachment of any of a variety of compounds using coupling techniques known in the art. This includes but is not limited to enzymatic means, oxidative substitution, chelation, etc., as used, for example, in the attachment of a radioisotope for immunoassay purposes.

The chemical linkage or coupling of a compound to the molecule could be directed to a site that does not participate in idiotype binding, for example, the Fc domain of the molecule. This could be accomplished by protecting the binding site of the molecule prior to performing the coupling reaction. For example, the molecule can be bound to the idiotype it recognizes, prior to the coupling reaction. After completion of coupling, the complex can be disrupted in order to generate a modified molecule with minimal effect on the binding site of the molecule.

The anti-idiotype antibodies, or fragments of antibody molecules of the invention, can be used as immunogens to induce, modify, or regulate specific cell-mediated tumor immunity. This includes, but is not limited to, the use of these molecules in immunization against syngeneic tumors.

Kits

The present invention further provides methods to identify the presence or expression of one of the polynucleotides or polypeptides of the present invention, or homolog thereof, in a test sample, using a nucleic acid probe or antibodies of the present invention, optionally conjugated or otherwise associated with a suitable label.

In general, methods for detecting a polynucleotide of the invention can comprise contacting a sample with a compound that binds to and forms a complex with the polynucleotide for a period sufficient to form the complex, and detecting the complex, so that if a complex is detected, a polynucleotide of the invention is detected in the sample. Such methods can also comprise contacting a sample under stringent hybridization conditions with nucleic acid primers that anneal to a polynucleotide of the invention under such conditions, and amplifying annealed polynucleotides, so that if a polynucleotide is amplified, a polynucleotide of the invention is detected in the sample.

In general, methods for detecting a polypeptide of the invention can comprise contacting a sample with a compound that binds to and forms a complex with the polypeptide for a period sufficient to form the complex, and detecting the complex, so that if a complex is detected, a polypeptide of the invention is detected in the sample.

In detail, such methods comprise incubating a test sample with one or more of the antibodies or one or more of the nucleic acid probes of the present invention and assaying for binding of the nucleic acid probes or antibodies to components within the test sample.

Conditions for incubating a nucleic acid probe or antibody with a test sample vary. Incubation conditions depend on the format employed in the assay, the detection methods employed, and the type and nature of the nucleic acid probe or antibody used in the assay. One skilled in the art will recognize that any one of the commonly available hybridization, amplification or immunological assay formats can readily be adapted to employ the nucleic acid probes or antibodies of the present invention. Examples of such assays can be found in Chard, T., An Introduction to Radioimmunoassay and Related Techniques, Elsevier Science Publishers, Amsterdam, The Netherlands (1986); Bullock, G. R. et al., Techniques in Immunocytochemistry, Academic Press, Orlando, Fla. Vol. 1 (1982), Vol. 2 (1983), Vol. 3 (1985); Tijssen, P., Practice and Theory of immunoassays: Laboratory Techniques in Biochemistry and Molecular Biology, Elsevier Science Publishers, Amsterdam, The Netherlands (1985). The test samples of the present invention include cells, protein or membrane extracts of cells, or biological fluids such as sputum, blood, serum, plasma, or urine. The test sample used in the above-described method will vary based on the assay format, nature of the detection method and the tissues, cells or extracts used as the sample to be assayed. Methods for preparing protein extracts or membrane extracts of cells are well known in the art and can be readily be adapted in order to obtain a sample which is compatible with the system utilized.

In another embodiment of the present invention, kits are provided which contain the necessary reagents to carry out the assays of the present invention. Specifically, the invention provides a compartment kit to receive, in close confinement, one or more containers which comprises: (a) a first container comprising one of the probes or antibodies of the present invention; and (b) one or more other containers comprising one or more of the following: wash reagents, reagents capable of detecting presence of a bound probe or antibody.

In detail, a compartment kit includes any kit in which reagents are contained in separate containers. Such containers include small glass containers, plastic containers or strips of plastic or paper. Such containers allows one to efficiently transfer reagents from one compartment to another compartment such that the samples and reagents are not cross-contaminated, and the agents or solutions of each container can be added in a quantitative fashion from one compartment to another. Such containers will include a container which will accept the test sample, a container which contains the antibodies or probes used in the assay, containers which contain wash reagents (such as phosphate buffered saline, Tris-buffers, etc.), and containers which contain the reagents used to detect the bound antibody or probe. Types of detection reagents include labeled nucleic acid probes, labeled secondary antibodies, or in the alternative, if the primary antibody is labeled, the enzymatic, or antibody binding reagents which are capable of reacting with the labeled antibody. One skilled in the art will readily recognize that the disclosed probes and antibodies of the present invention can be readily incorporated into one of the established kit formats which are well known in the art.

### EXAMPLES

### Example 1

IGSF9 Expression

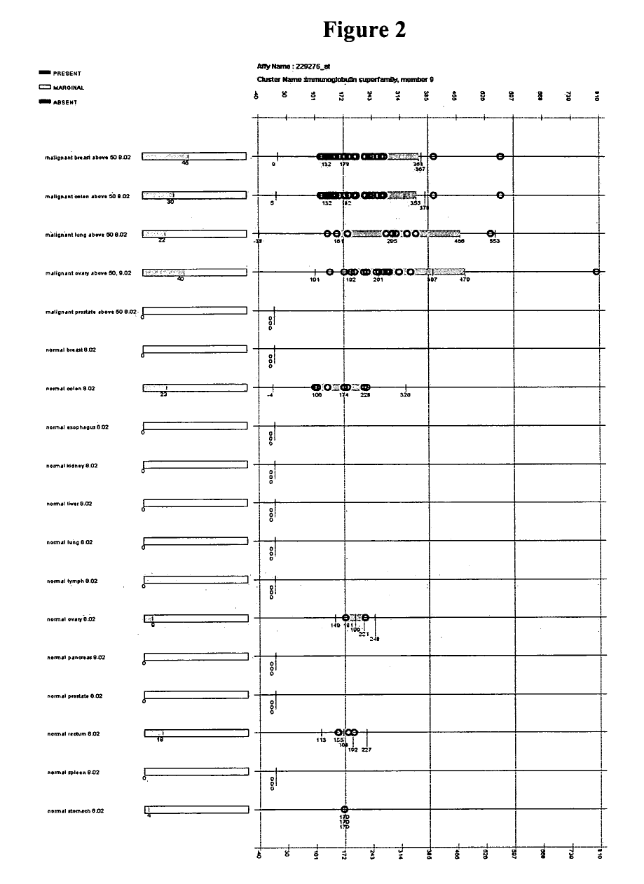

IGSF9 gene expression was examined in a variety of normal and neoplastic tissues. FIG. 2 is an ‘electronic Northern’ depicting the gene expression profile of this gene as determined using the Gene Logic datasuite. The values along the y-axis represent expression intensities in Gene Logic units. Each blue circle on the figure represents an individual patient sample. The bar graph on the left of the figure depicts the percentage of each tissue type found to express the gene fragment. The total number of samples for each tissue type is as follows: malignant breast (60); malignant colon (91); malignant lung (40); malignant ovary (37); malignant prostate (26); normal breast (30); normal colon (30); normal esophagus (17), normal kidney (27); normal liver (19); normal lung (34); normal lymph node (9); normal ovary (22); normal pancreas (18); normal prostate (21); normal rectum (22); normal spleen (9); normal stomach (21).

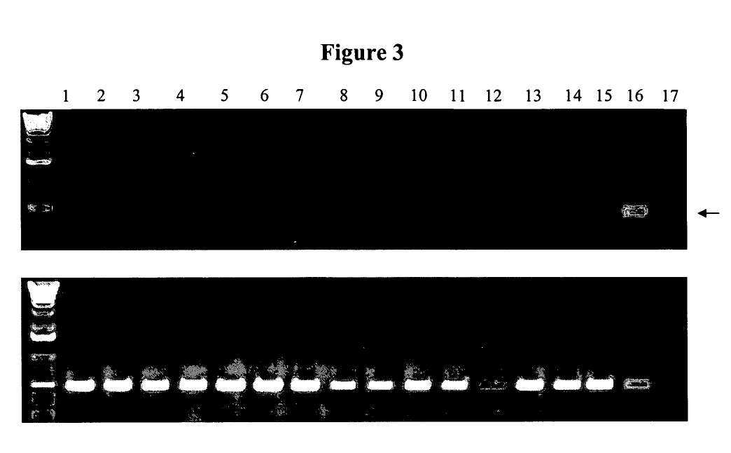

In addition, the expression of IGSF9 in normal and malignant human tissues was further investigated by PCR experiments using commercially available human cDNA panels and cDNA samples prepared in-house from human tissues and cell lines. The results of these experiments are presented below in FIGS. 3-7. The following PCR primers were synthesized and used in all experiments.

| | |
|---|---|
| 5′-TCTTATCTTCTCTCCGACCGGGAAG-3′ | (SEQ ID NO:30) |
| 5′-GCCACAGGGCTGATGTCTTCAATGC-3′ | (SEQ ID NO:31) |

The sequence of these primers is contained in the portion of IGSF9 present in IMAGE clone # 2013096/ATCC catalog # 3068496, plasmid DNA from which was used as a positive control in each experiment. These primers amplify a. PCR product of 387 bp from any cDNA template containing the IGSF9 gene. Expression of Glyceraldehyde 3-phosphate dehydrogenase (GAPDH) is measured in all experiments as a control for cDNA integrity. GAPDH is a housekeeping gene expressed abundantly in all human tissues. Primers used for amplification of the GAPDH gene are:

| | |
|---|---|
| 5′-ACCACAGTCCATGCCATCAC-3′ | (SEQ ID NO:32) |
| 5′-TCCACCACCCTGTTGCTGTA-3′ | (SEQ ID NO:33) |

These primers amplify a 482 bp product from any cDNA template encoding the GAPDH gene. In all cases, positive and negative controls are also included; the positive control is plasmid DNA for IMAGE clone 4762143, the negative control is water (no template).

FIG. 3 shows the expression of IGSF9 in normal tissues, as determined using Clontech's Human Multiple Tissue cDNA panels (BD Biosciences, catalog #s K1420-1 and K1421-1) The upper panel shows IGSF9 expression, while the lower panel shows expression of GAPDH. The cDNA samples present in each lane are as follows: (1) brain, (2) placenta, (3) lung, (4) liver, (5) skeletal muscle, (6) kidney, (7) pancreas, (8) spleen, (9) thymus, (10) prostate, (11) testis, (12) ovary, (13) small intestine, (14) colon, (15) peripheral blood leukocytes, (16) positive control, and (17) negative control. The arrowhead on the right of the figure denotes the anticipated size of the IGSF9 PCR product. The data in this figure indicates that IGSF9 is expressed weakly in normal liver, pancreas, prostate, testis and colon, and is absent from all other normal tissues.

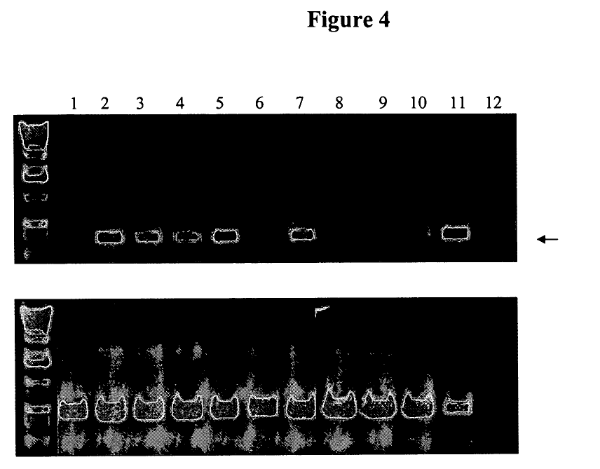

Shown in FIG. 4 is IGSF9 expression in a panel of human ovarian tumor samples and two ovarian tumor cell lines. The ovarian tumor samples were obtained from the Cooperative Human Tissue Network (CHTN); the cell lines Ovcar-3 and PA1 were obtained from the American Type Culture Collection (ATCC, Rockville Md.). RNA was isolated from each sample and cell line using Qiagen's RNeasy kit (catalog # 75162). cDNA was prepared from total RNA using Invitrogen's cDNA synthesis system (catalog # 11904-018.) The upper panel shows IGSF9 expression, the lower panel shows GAPDH expression. The numbers above each lane correspond to ovarian tumor samples as follows: (1) moderately differentiated cystadenocarcinoma, (2) poorly differentiated papillary serous adenocarcinoma, (3) poorly differentiated papillary serous adenocarcinoma, (4) poorly differentiated endometriod adenocarcinoma, (5) papillary serous adenocarcinoma, (6) endometriod adenocarcinoma, (7) poorly differentiated adenocarcinoma, (8) poorly differentiated papillary serous adenocarcinoma, (9) Ovcar-3 cell line, (10) PA-1 cell line, (11) positive control, and (12) negative control. The arrowhead on the right of the figure denotes the anticipated size of the IGSF9 PCR product. The data in this panel indicates that IGSF9 is expressed 7 of the 8 tumor samples, with strong expression in 5 of these. It is also expressed in both of the ovarian tumor cell lines.

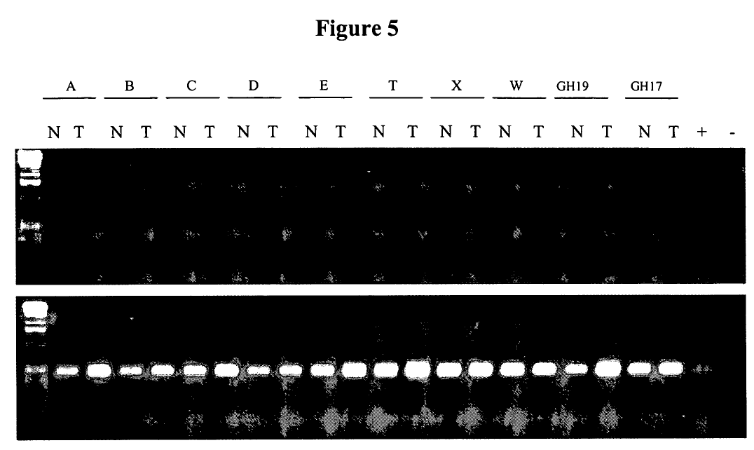

FIG. 5 shows expression of IGSF9 in breast tumor samples and matched normal breast samples. Expression in breast tissue was determined using Clontech's Human Breast Matched cDNA pair panel (BD Biosciences, catalog # K1432-1, first 5 sample sets) and 5 in-house matched samples obtained from Grossmont Hospital, La Mesa Calif. RNA was isolated from each sample using TRIzol Reagent (Invitrogen, catalog # 15596026). cDNA was prepared from total RNA using Gibco BRL cDNA synthesis system (Life Technologies, catalog # 18267-021). The upper gel shows IGSF9 expression; lower gel shows GAPDH expression. (N) normal tissue, (T) tumor tissue. The tumor samples are as follows: (Patient A) infiltrating ductal carcinoma; (patient B) infiltrating ductal carcinoma, (patient C) tubular adenocarcinoma; (patient D) infiltrating ductal carcinoma, (patient E) infiltrating ductal carcinoma, (patient T) high grade in situ & invasive ductal carcinoma, (patient X) ductal adenocarcinoma, (patient W) mixed ductal and lobular adenocarcinoma, (patient GH19) high grade invasive ductal carcinoma, (patient GH17) low grade intraductal carcinoma. The arrowhead on the right of the figure denotes the anticipated size of the IGSF9 PCR product. The data presented here indicates that IGSF9 is expressed in 8 of 10 breast tumor samples, but only 4 of 10 normal samples.

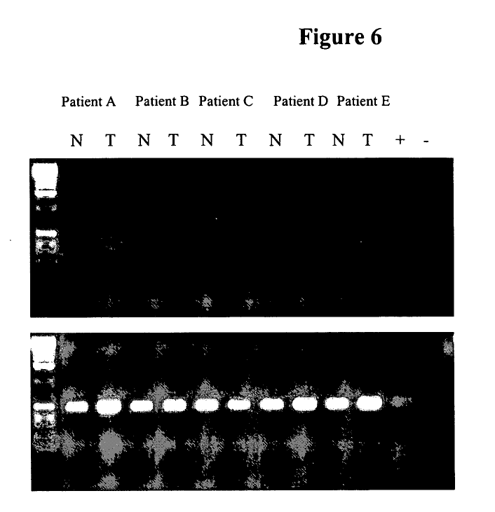

IGSF9 expression in lung tumors is shown in FIG. 6. Expression was determined using Clontech's Human Lung Matched cDNA Pair Panel (BD Biosciences, catalog # K1434-1). The upper panels shows IGSF9 expression, while the lower panel shows GAPDH expression. (N) normal sample; (T) tumor sample. The tumor samples analyzed were as follows: (Patient A) infiltrating ductal carcinoma, (patient B) squamous cell keratinizing carcinoma, (patient C) adenosquamous carcinoma, (patient D) keratinizing squamous cell carcinoma, (patient E) squamous cell carcinoma. The arrowhead on the right of the figure denotes the anticipated size of the IGSF9 PCR product. The data shown here indicates that IGSF9 is present in all 5 lung tumor samples but only in 2 of 5 normal samples.

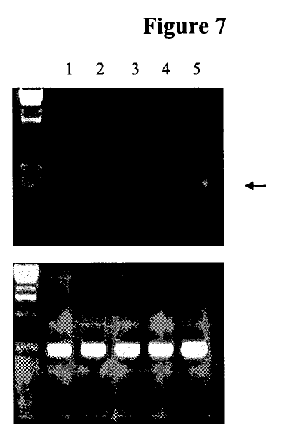

IGSF9 expression in colon tumors is shown in FIG. 7. Colon tumor samples were obtained from Grossmont Hospital in La Mesa, Calif. Colorectal cancer cell line HCT116 was obtained from the American Type Culture Collection (ATCC, Rockville, Md.) RNA was isolated from each sample and cell line using Qiagen's RNeasy kit (catalog # 75162). cDNA was prepared from total RNA using Gibco BRL cDNA synthesis system (Life Technologies, catalog #18267-021). The upper panel shows IGSF9 expression, while the lower panel shows GAPDH expression. Samples are as follows: (1) grade 3 adenocarcinoma, (2) grade 2 adenocarcinoma, (3) grade 1 adenocarcinoma, (4) grade 2 adenocarcinoma, (5) colorectal cancer cell line HCT116. The arrowhead on the right of the figure denotes the anticipated size of the IGSF9 PCR product. The data in this figure indicates that IGSF9 is expressed in the colon tumor cell line HTC116, and may also be expressed weakly in at least 1 of the 4 tumor samples.

Taken together, the data presented here indicates that IGSF9 is expressed at significant levels in multiple ovarian, breast, lung and colon tumor samples. IGSF9 may therefore represent a pancarcinoma antigen and a suitable target for tumor therapy in any of the above mentioned indications.

### Example 2

Expression of IGSF9 in Human Tumor Cells Determined by RT-PCR

The expression of IGSF9 in a collection of human tumor cell lines obtained from ATCC (Manassas, Va.) and the Arizona Cancer Center (Tucson, Ariz.) was investigated by RT-PCR. The results of this experiment, depicted in FIG. 8, indicate that IGSF9 is expressed in a number of different tumor cell lines.

The following tumor cell lines were used:

Pancreatic: PANC-1.

Breast: ZR-75-1, MDA-MB468, MAD-MB231, ME-180, UACC812.

Ovarian: UACC326.

Lung: A549 (NSCLC), NCI-H69(small cell), NCI-H1299(NSCLC), NCI-H2126(NSCLC).

Colon: HT 29, LoVo, SW 620, Colo201, Colo205, Colo320.

RNA was isolated from each cell line using the Qiagen RNeasy® kit, and cDNA was subsequently prepared from total RNA using Invitrogen's cDNA synthesis system. The result of the PCR experiment is interpreted in FIG. 8, in which relative expression of IGSF9 in each sample is presented as the ratio of the intensity of IGSF9 versus the intensity of the internal control glyceraldehyde 3-phosphate dehydrogenase (GAPDH).

The following PCR primers were synthesized and used in all experiments.

| | |
|---|---|
| 5′-TCTTATCTTCTCTCCGACCGGGAAG-3′ | (SEQ ID NO:34) |
| 5′-GCCACAGGGCTGATGTCTTCAATGC-3′ | (SEQ ID NO:35) |

These primers amplify a PCR product of 387 bp from any cDNA template containing the IGSF9 gene. Expression of GAPDH was measured in all experiments as a control for cDNA integrity. Primers used for amplification of the GAPDH gene were:

| | |
|---|---|
| 5′-ACCACAGTCCATGCCATCAC-3′ | (SEQ ID NO:36) |
| 5′-TCCACCACCCTGTTGCTGTA-3′ | (SEQ ID NO:37) |

These primers amplify a 482 bp product from any cDNA template encoding the GAPDH gene.

### Example 3

Generation of Stable Mammalian Cell Lines Expressing IGSF9 Constructs

Two alternate forms of IGSF9 were identified in public databases, herein referred to as ‘short form’ and ‘long form’ IGSF9. The long form of IGSF9 is an alternately spliced variant containing a 17 amino acid insertion in the extracellular domain located between 2 Ig domains. The nucleotide and protein sequences of the IGSF9 short form are shown in FIGS. 1A and 1B. FIGS. 9E and 9F depict the nucleotide and protein sequences of the long form of IGSF9, respectively.

Full length cDNAs encoding both short and long forms if IGSF9 were constructed from commercially available EST plasmids using standard molecular cloning techniques and synthetic oligonucleotide primers. Full length clones were then inserted into proprietary mammalian expression vectors (described in U.S. Pat. Nos. 5,648,267, 5,733,779, 6,017,733, and 6,159,730, although commercially available vectors such as pIND/hygro available from Invitrogen; pWLNEO, pSV2CAT, pOG44, pXT1 and pSG available from Stratagene; and pSVK3, pBPV, pMSG and pSVL available from Pharmacia; and the like could be used). Soluble forms of both short and long form IGSF9 were also constructed by genetically fusing the cDNAs encoding the extracellular domains of the molecules to cDNA encoding human IgG1 Fc domain (immunoadhesins.) The extracellular domains of short and long form IGSF9 were generated by PCR methodology using the full length genes as templates. These constructs were then inserted into a proprietary mammalian expression vector containing the IgG1 Fc gene sequence. Cloning resulted in an in-frame fusion of the IGSF9 extracellular domain with the N-terminus of the IgG1 Fc (see FIG. 9 for all sequences.)

All of the above constructs were subsequently used to generate stably transfected Chinese hamster ovary (CHO) cell lines. Briefly, expression constructs were transfected into DHFR-CHO DG44 cells (Urlaub et. al., 1985. Som. Cell. Mol. Gen., 12:555-566) by electroporation. Cells were washed, counted and resuspended in ice cold SBS buffer (7 mM NaPO₄, 1 mM MgCl₂, 272 mM sucrose, pH 7.4.) Plasmid DNA was linearized by PacI restriction digestion and 1 or 0.5 ug/ml DNA mixed with 4×10⁶ DG44 cells and electroporated. Cells were seeded into 96-well microtiter culture plates and cell lines were selected for G418 resistance in CHO S SFM II media (Gibco) supplemented with hypoxanthine+thymidine (HT, Gibco). Wells from the plates transfected with the lowest concentration of DNA and exhibiting robust cellular growth were screened for surrogate marker expression by ELISA (B7Ig in the case of full length constructs, and CTLA4Ig for immunoadhesin constructs). The highest producing immunoadhesin cell lines were expanded into spinner cultures, and immunoadhesin molecules were purified from culture supernatants by protein A affinity chromatography and subsequently used as immunogens for murine monoclonal antibody production (see Example 4).

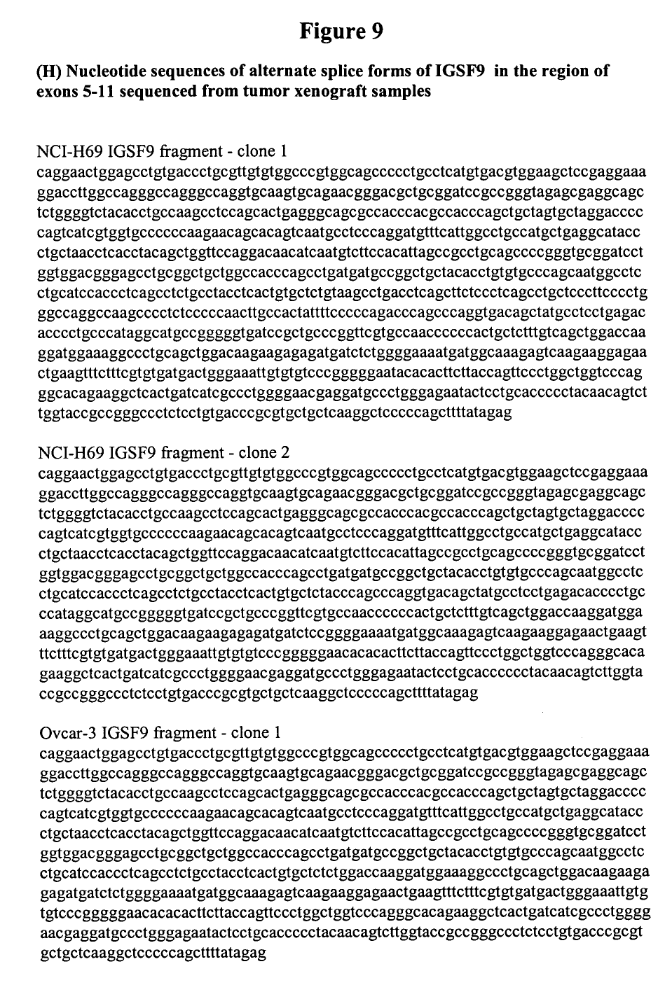

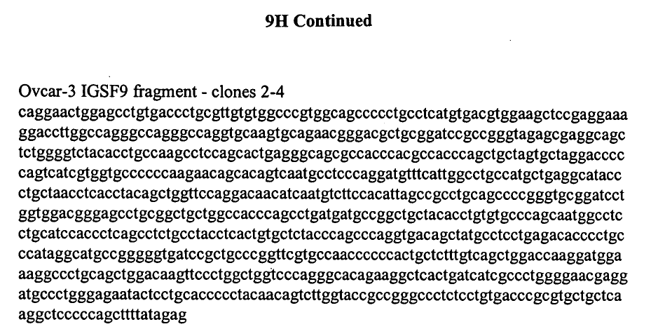

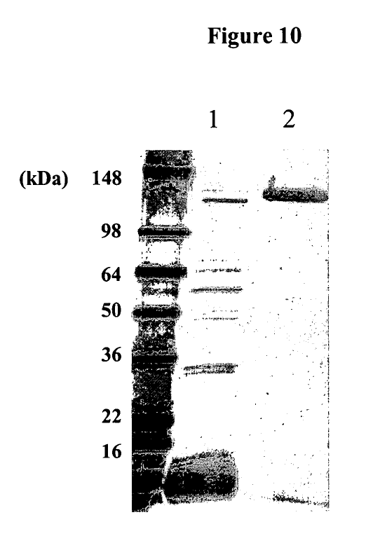

FIG. 10 shows an SDS-PAGE analysis of purified immunoadhesins. Material was purified from 10 liters of culture supernatant. Proteins were visualized by coomassie blue staining. A robust band of the predicted molecular weight is seen for the long form of IGSF9 only (lane 2). The short form (lane 1) gives rise to multiple degradation products. The data in FIG. 10 indicates that recombinant IGSF9 molecules can be expressed successfully at high levels in mammalian cells.

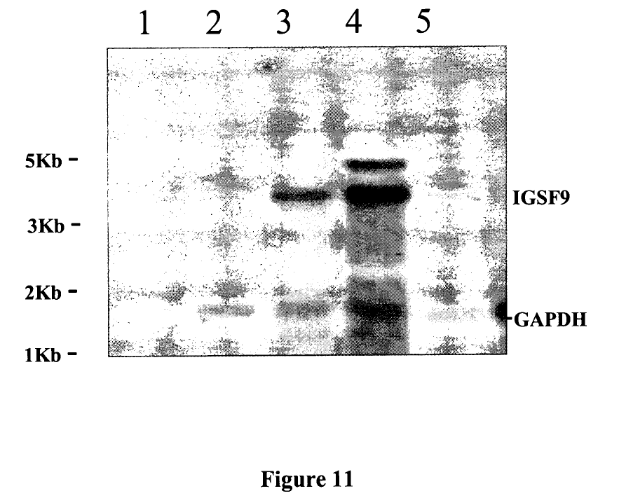

The cell lines expressing the highest levels of full length IGSF9 constructs were amplified in 5 nM methotrexate (MTX) and subsequently 50 nM MTX. Briefly, cells were seeded at a density ranging from 1.5 cells/plate to 3000 cells/plate in two-fold increments and cultured in media containing 5 nM MTX or 50 nM MTX for two weeks. The surviving cells were screened for surrogate marker expression by ELISA, and the highest producing clones were expanded into spinner cultures. Expression of IGSF9 message in resultant cell lines was confirmed by RT-PCR and Northern blotting. A representative Northern blot is shown in FIG. 11. For Northern analysis, total RNA was extracted from 1×10⁸ cells using the Qiagen RNeasy® Maxi kit following the manufacturer's protocol. mRNA was isolated using Qiagen Oligotex® mRNA Direct Midi/Maxi kit using the recommended batch protocol. 3 μg of mRNA was separated on a 1% agarose gel containing 3% formaldehyde and blotted according to standard procedures. Nucleotide probes specific for the extracellular region of IGSF9, along with a GAPDH control probe were labeled with digoxygenin (DIG) by PCR using a DIG-labeling nucleotide mix according to the manufacturer's instructions (Roche). The blot was hybridized at 50° C. in DIG Easy Hyb solution (Roche) using the IGSF9 probes at equal concentrations for a total of 50 ng/ml and the GAPDH probe at 15 ng/ml. The blot was washed and detected using a DIG wash and block detection system according to the manufacturer's instructions (Roche). The blot was subsequently exposed to film for approximately 16 hours. One major product of the expected size is seen in FIG. 11 in lanes 2-5, as indicated on the figure. The appearance of a second, larger transcript is possibly due to run-on transcription. The data presented in this figure confirms that recombinant IGSF9 molecules are expressed at detectable levels in mammalian cells.

### Example 4

Generation of anti-IGSF9 Monoclonal Antibody 8F3

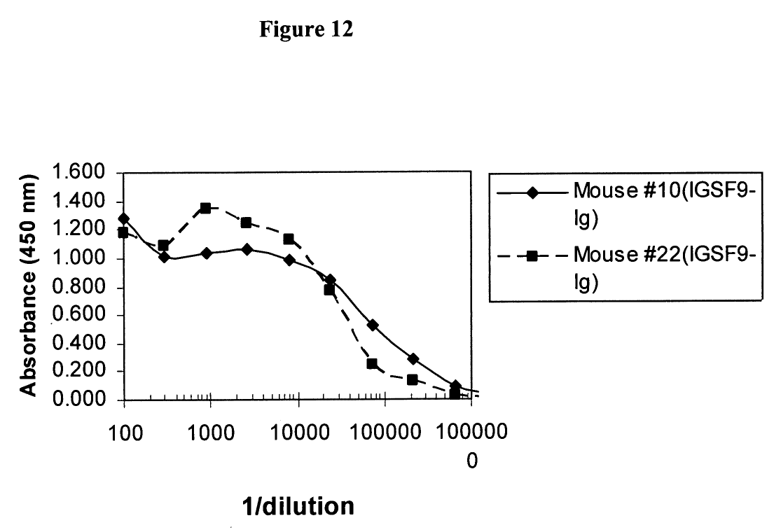

Monoclonal antibodies were produced by injecting 6-8 week old male BALB/c mice initially with a cDNA construct encoding the short form soluble IGSF9-Ig five times using a gene gun. Mice were subsequently boosted with short form IGSF9-Ig fusion protein purified from the supernatant of a stably expressing CHO cell line (see the preceding Example) by protein-A affinity chromatography. Mice were injected with the purified protein in a rapid immunization technique consisting of five sets of twelve injections over a period of eleven days. Mice were bled on day 12, and the titer of IGSF9 specific antibodies was determined by ELISA on 96 well plates coated with purified short form IGSF9-Ig. On day 13, spleens from mice exhibiting the highest titer were removed and fused to mouse myeloma Sp2/0 cells following standard immunological techniques (Kohler, G. and Milstein, C. 1975. Nature 256, p 495). FIG. 12 depicts a representative ELISA measuring IGSF9 reactivity in serial dilutions of sera from two mice immunized as described above.

All hybridomas were initially screened for reactivity against short form IGSF9-Ig by ELISA and all positives were then screened against irrelevant Ig fusion proteins to rule out any cross-reactive antibodies. The highest producing clones were subcloned by limiting dilution and ultimately expanded into spinner flasks. Antibodies were purified from culture supernatants by protein-A affinity chromatography after 10-12 days, and isotype determination was performed using a mouse immunoglobulin ELISA kit (Pharmingen) according to the manufacturers instructions. One monoclonal antibody, referred to as 8F3, was selected for further studies based on its high titer and binding specificity for IGSF9. Examples 5 and 6 describe experiments using this antibody to examine expression of IGSF9 in a variety of relevant tissues.

### Example 5

IGSF9 Expression on Stable Cell Lines and Tumor Cell Lines Detected Using Monoclonal Anti-IGSF9 Antibodies

IGSF9 Surface Expression in Stably Transfected CHO Cells as Measured by Flow Cytometry

Expression of recombinant IGSF9 molecules on the surface of stably transfected CHO cells was confirmed by flow cytometry using the biotinylated anti-IGSF9 monoclonal antibody 8F3. The antibody was biotinylated using an ECL protein biotinylation module according to manufacturer's instructions (Amersham Pharmacia).

For flow cytometry, cells were harvested and washed twice with PBS. 3-5×10⁵ cells were subsequently aliquoted into 96 well round-bottom plates and washed with FACS buffer (PBS containing 10% normal goat serum, 0.2% BSA, and 0.1% NaN₃) three times. Cell pellets were resuspended in 100 μl FACS buffer along with 100 μl of primary antibodies (biotinylated 8F3 or isotype control) at 10 μg/ml and incubated on ice for 1 hour. The plate was then centrifuged and the supernatants needle aspirated. The cell pellets were then washed an additional two times with FACS buffer as described above. Cells were subsequently incubated with a 1:500 dilution of Streptavidin-PE (BD Pharmingen) for an additional hour on ice, after which time cells were washed as above then resuspended in 500 μl FACS buffer containing 5 μl propidium iodide to separate live from the dead cells. Fluorescence intensity was measured using a Becton Dickinson FACScalibur cytometer, gated for HLA-APC positive and propidium iodide negative cell populations.

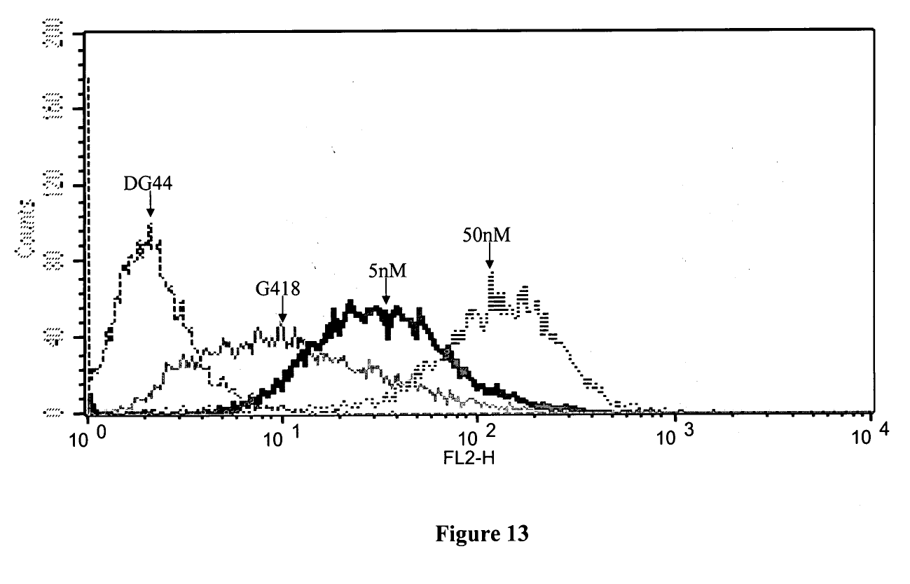

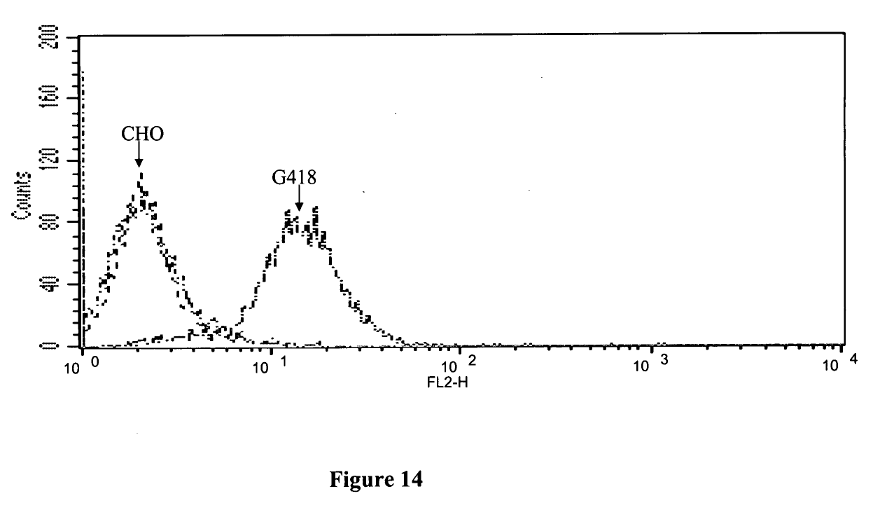

FIGS. 13 and 14 depict flow cytometry analyses of both short and long forms of IGSF9 expression in stable CHO transfectants. The data in these figures indicates that both forms are expressed on the surface of the transfected cells, and increasing MTX amplification of the short form transfectant results in increased surface expression of the molecule.

IGSF9 Surface Expression on Tumor Cell Lines as Measured by Flow Cytometry

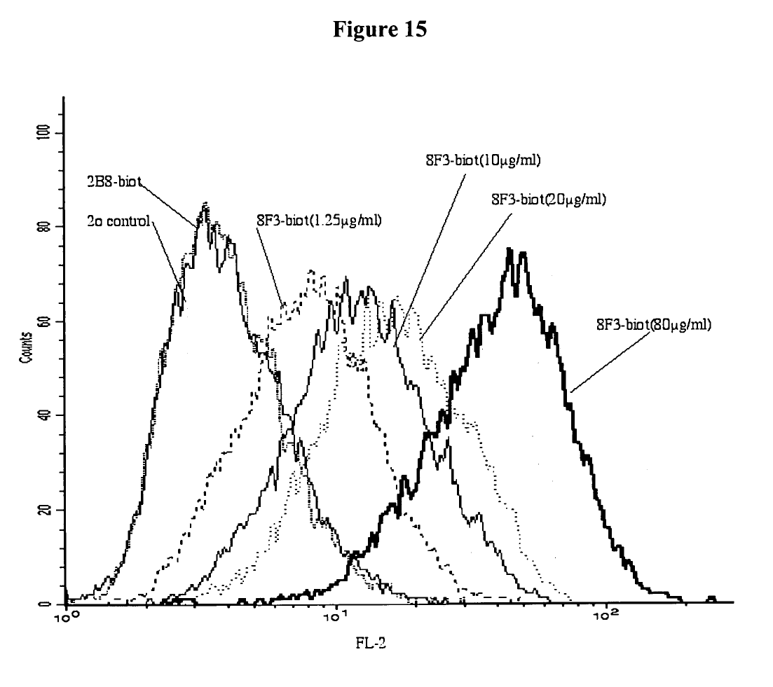

Endogenous surface expression of IGSF9 in the human lung tumor cell line NCI-H69 was measured by flow cytometry essentially as described above, except that multiple concentrations of the primary antibody 8F3 were tested. The results of this experiment are shown in FIG. 15. This experiment demonstrates that endogenously expressed IGSF9 is found on the surface of human tumor cell lines.

IGSF9 Expression in Tumor Cell Lines as Measured By Western Blotting

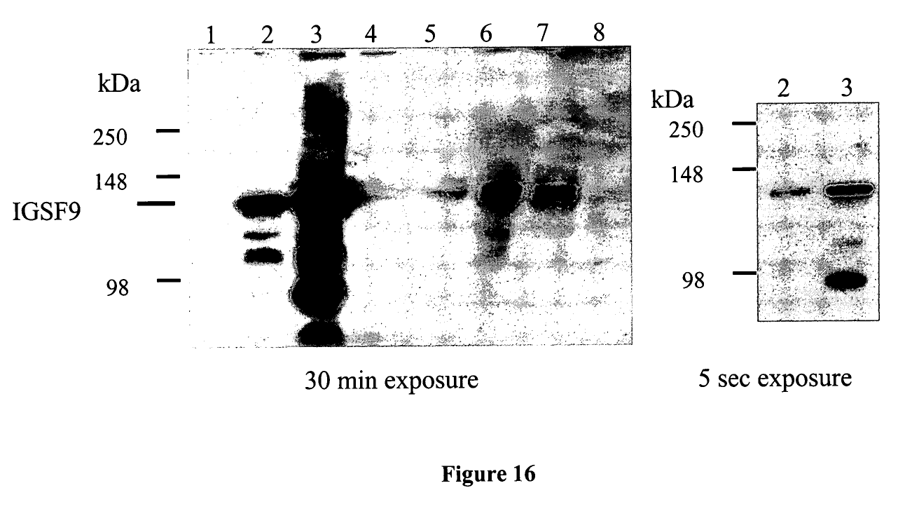

Immunoblotting experiments using protein lysates from human tumor cell lines probed with the anti-IGSF9 monoclonal antibody 8F3 confirm that IGSF9 protein is expressed at detectable levels in a number of human tumor cell lines. This data is represented in FIG. 16. Total protein lysates were prepared by direct cell lysis in SDS gel sample buffer and resolved by SDS-PAGE. The protein concentrations of the lysates were determined using the DC Protein Assay kit (BiORad) according to the manufacturer's instructions. The cell lysates were resolved by SDS-PAGE (6% acrylamide gel), transferred to a PVDF membrane, and immunoblotted using purified anti-IGSF9 mAb (8F3; 10 μg/ml) overnight at 4° C. followed by incubation with horseradish peroxidase (HRP)-conjugated anti-mouse IgG secondary antibody (BiORad) at a 1:1,000 dilution. The immunoblot was developed using ECL reagent (Amersham Pharmacia) according to the manufacturer's instructions.

IGSF9 Expression on the Surface of Tumor Cells as Measured by Fluorescence Microscopy

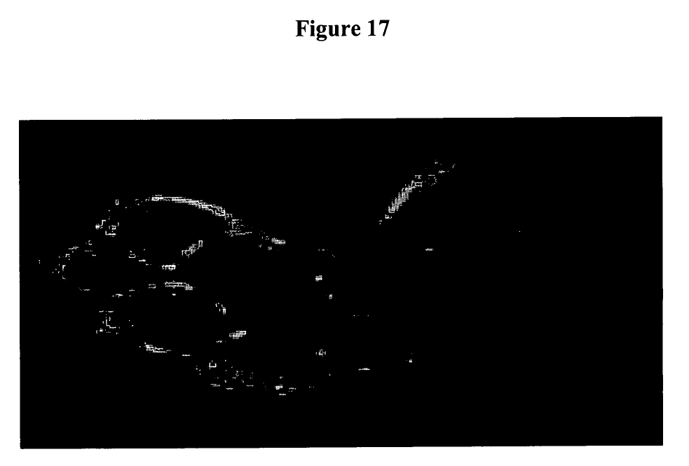

ZR-75-1 breast tumor cells grown on poly L-Lysine-coated glass coverslips were incubated for 16 hours with the anti-IGSF9 monoclonal antibody 8F3 (10 μg/ml). The cells were washed with PBS and fixed using ice-cold methanol. The fixed cells were blocked in blocking buffer (3% goat serum, 0.5% BSA in PBS) and incubated for 45 minutes at room temperature with DAPI (0.5 μg/ml) and Alexa488-Goat anti-mouse secondary antibody (Molecular Probes) at a dilution of 1:2000. The cells were washed with PBS, mounted on glass slides using the ProLong! Antifade Kit (Molecular Probes) and examined using a BiORad Radiance 2100 confocal microscope system (60× objective). The results of this experiment are depicted in FIG. 17. This figure demonstrates surface staining of the breast tumor cells.

Taken together, the data presented in FIGS. 13-17 demonstrate that monoclonal antibody 8F3 has reactivity toward IGSF9, and serve to confirm that IGSF9 is a cell surface protein. These data also support the hypothesis that IGSF9 may be a suitable immunotherapy target for human tumors, as it is found expressed at significant levels on the surface of human tumor cell lines.

### EXAMPLE 6

IGSF9 Expression in Murine Tumor Xenografts

Murine tumor xenografts were generated as follows: tumor cell lines NCI-H69 (lung) and ZR-75-1 (breast), LS174T (colon) and Ovcar-3 (ovary) cultured in vitro were harvested and cell aggregates dissociated by passing the cell suspension through a syringe with a 22 gauge needle. Cells were washed, counted, and resuspended in PBS.

2-10×10⁶ cells/100 μL were injected subcutaneously (s.c.) on the right flank of nude mice. Tumor masses were excised after 4-8 weeks of growth. For in vivo repassaging, 2 mm tumor sections were reintroduced s.c. into the flank of nude mice and allowed to grow for 4-8 weeks.

IGSF9 Surface Expression on Murine Tumor Xenografts and Cell Lines as Measured by Flow Cytometry Using Anti-IGSF9 Monoclonal Antibodies

For flow cytometry analysis, fresh tumor samples were minced and digested at 37° C. for one hour with a collagenase solution containing 5% BSA and 0.05% NaN₃. Live cells were separated from dead cells and other debris by density gradient centrifugation. Cells were then plated into 96-well round bottom plates and processed for flow cytometry as described in Example 5. Cells grown in culture were detached using a non-enzyrnatic buffer, washed, plated into 96 well plates, and processed as described previously.

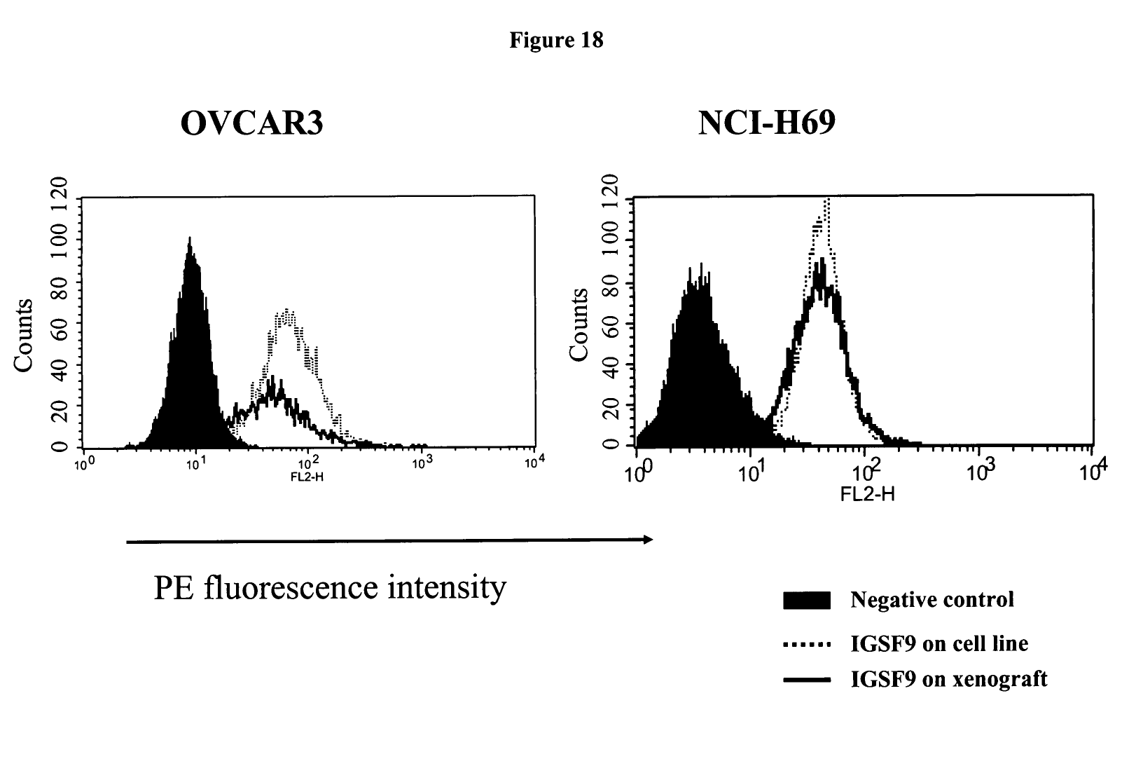

FIG. 18 shows a representative FACS experiment measuring IGSF9 expression in NCI-H69 and Ovcar-3 murine tumor xenografts and cultured cells. The data in FIG. 18 indicates that IGSF9 is expressed on the surface of cells grown both in culture or in in vivo passaged cells derived from murine xenografts. Expression of IGSF9 on the surface of human tumor cells growing in vivo further supports the idea that IGSF9 is a suitable therapeutic target.

IGSF9 Message in Murine Tumor Xenografts is Detected by RT-PCR

Expression of IGSF9 in tumor xenograft samples was measured by RT-PCR using human IGSF9-specific and GAPDH control primers. Xenograft samples were generated and excised as described previously. Total RNA was isolated from 0.25 g tissue samples using the Qiagen RNeasy® kit, treated with DNase, and purified using Qiagen minElute® columns. cDNA was synthesized using an oligo-dT primer and Invitrogen's Super Script First-Strand Synthesis system. PCR was performed under standard conditions. The PCR primers used to amplify IGSF9 were as follows:

| | |
|---|---|
| Forward primer | |
| 5′-GTGGGCCGGGGGCTGCAAGGCCAG-3′ | (SEQ ID NO:38) |
| Reverse primer | |
| 5′-AGCAGACAAGACGATTTCGCTGAA-3′ | (SEQ ID NO:39) |

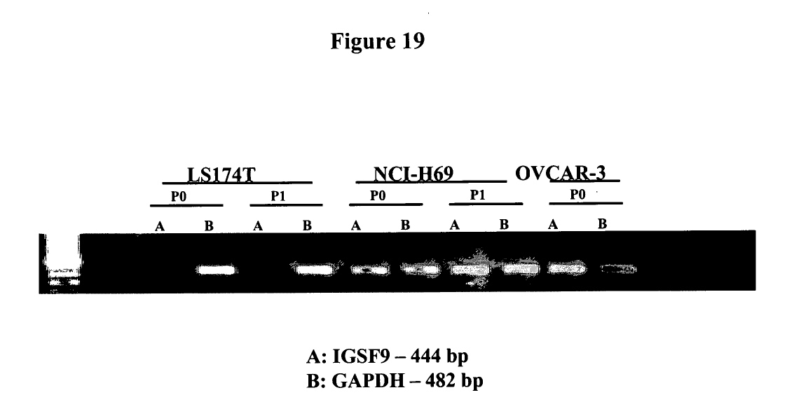

The results of a representative RT-PCR experiment are shown in FIG. 19. IGSF9 message was detected in two in vivo passages (P0 and P1) of both LS174T and NCI-H69 tumor cell lines, and in at least one passage (P0) of Ovcar-3 cells derived from murine xenografts.

Alternate Splice Forms of IGSF9 are Expressed in Murine Xenograft Tumors

Sequence analysis of PCR products obtained from murine xenograft samples indicated that multiple isoforms of IGSF9 are expressed in the tumor derived cells. RT-PCR analysis was carried out as described above, using primers designed to flank the region of IGSF9 where the short and long isoforms described earlier diverge in sequence (in exon 9) PCR primers were as follows:

| | |
|---|---|
| Forward primer | |
| 5′-CAGGAACTGGAGCCTGTGACCCT-3′ | (SEQ ID NO:40) |
| Reverse primer | |
| 5′-CTCTATAAAAGCTGGGGGAGCCTT-3′ | (SEQ ID NO:41) |

PCR products were shotgun cloned using the pCR4-TOPO TA cloning system (Invitrogen) and inserts were sequenced using an ABI automated DNA sequencer. Two novel isoforms were identified in clones derived from NCI-H69 xenografts, and an additional different novel isoform was identified in clones derived from Ovcar-3 xenografts.

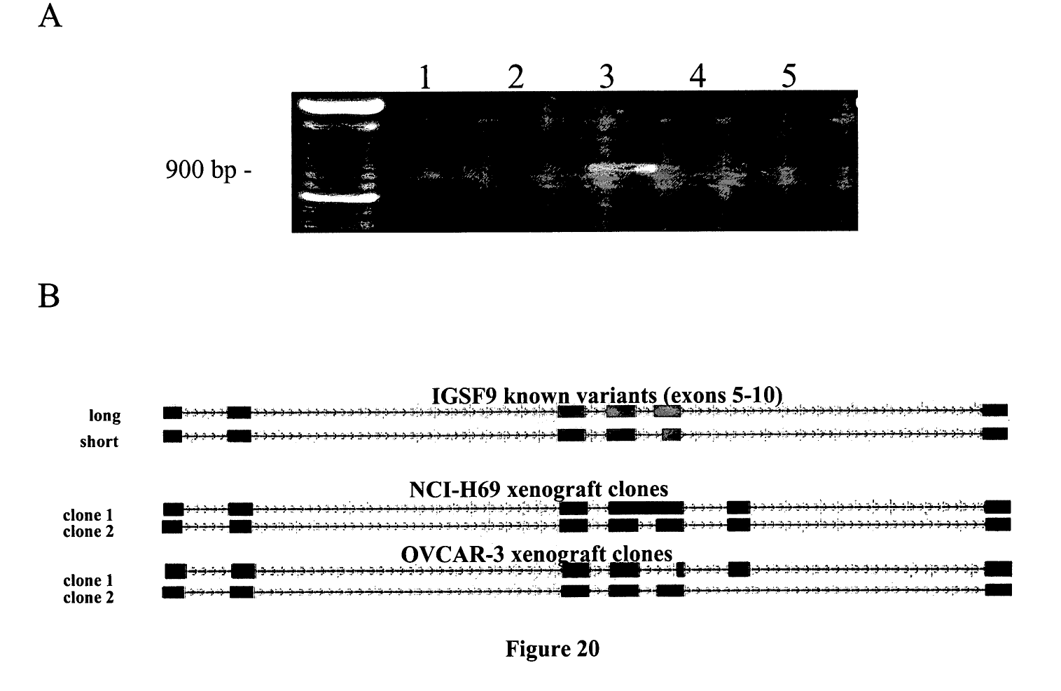

All novel isoforms follow the AG/GT splicing rule, suggesting that they are true splice variants (Breathnach R. et al, 1978. Proc. Natl. Acad. Sci. USA 75; 4853-7.) A representative PCR gel is depicted in FIG. 20, along with a schematic representation of the exons of IGSF9 affected by the alternate splicing. In-frame translation of each nucleotide sequence obtained predicts that all novel sequences would produce a truncated protein lacking a transmembrane domain. An alignment of the actual nucleotide sequences obtained, along with their corresponding predicted protein sequences, is shown in FIG. 21. The partial nucleotide sequences were aligned with exons 5-10 of IGSF9 long form.

The sequencing data presented here indicates that multiple isoforms of IGSF9 may exist in human tumors, and many isoforms may represent potential immunotherapeutic targets.

### Example 7

LIV-1 Expression

FIG. 22 is an electronic Northern depicting the gene expression profile of this gene as determined using the Gene Logic datasuite. The values along the y-axis represent expression intensities in Gene Logic units. Each blue circle on the figure represents an individual patient sample. The bar graph on the left of the figure depicts the percentage of each tissue type found to express the gene fragment. The total number of samples for each tissue type is as follows: malignant breast (60); malignant colon (91); malignant lung (40); malignant ovary (37); malignant prostate (26); normal breast (30); normal colon (30); normal esophagus (17), normal kidney (27); normal liver (19); normal lung (34); normal lymph node (9); normal ovary (22); normal pancreas (18); normal prostate (21); normal rectum (22); normal spleen (9); normal stomach (21).

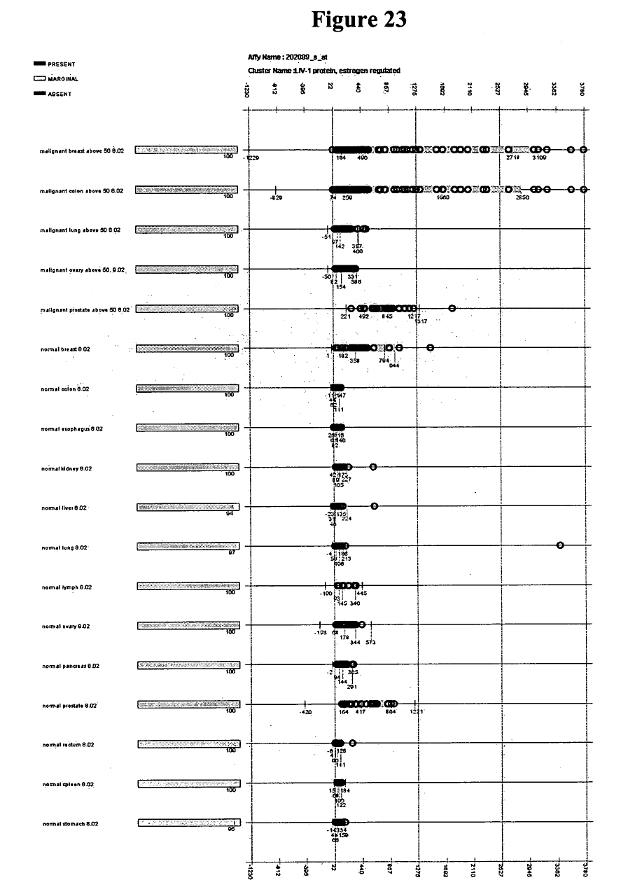

The expression of LIV-1 in normal and malignant human tissues was further investigated by PCR experiments using commercially available human cDNA panels and cDNA samples prepared in-house from human tissues and cell lines, as described in the previous example. The results of these experiments are presented in FIGS. 23-25. The following PCR primers were synthesized and used in all experiments:

| | |
|---|---|
| 5′-GGATGGTGATAATGGGTGATGGC-3′ | (SEQ ID NO:42) |
| 5′-GGTCACTAGCATCATTGTGCAGC-3′ | (SEQ ID NO:43) |

The sequence of these primers is contained in the portion of LIV-1 present in IMAGE clone # 4697878/ATCC catalog # 6645729, plasmid DNA from which was used as a positive control in each experiment. These primers amplify a PCR product of 360 bp from any cDNA template containing the LIV-1 gene. Expression of GAPDH is measured in all experiments as a control for cDNA integrity, as described in the previous example.

The LIV-1 primers amplify a 482 bp product from any cDNA template encoding the GAPDH gene. In all cases, positive and negative controls are also included; the positive control is plasmid DNA for IMAGE clone 4697878, the negative control is water (no template).

FIG. 23 shows expression of LIV-1 in normal tissues, as determined using Clontech's Human Multiple Tissue cDNA Panels (BD Biosciences, catalog #s K1420-1 and K1421-1). The upper panel shows LIV-1 expression, while the lower panel shows GAPDH expression. The cDNA samples present in each lane are as follows: (1) heart, (2) brain, (3) placenta, (4) lung, (5) liver, (6) skeletal muscle, (7) kidney, (8) pancreas, (9) negative control, and (10) positive control. The arrowhead on the right of the figure denotes the anticipated size of the LIV-1 PCR product. The data presented here indicates that LIV-1 is expressed weakly in normal brain, placenta, lung, liver and kidney, and to a slightly greater extent in normal pancreas.

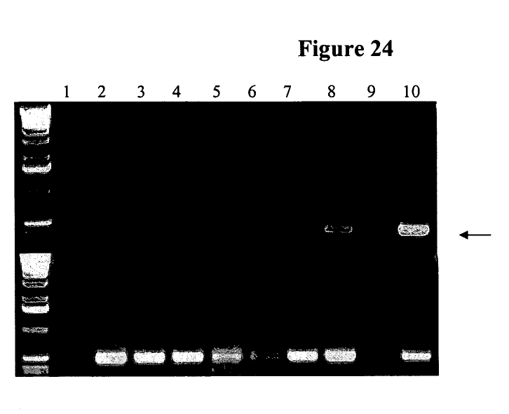

FIG. 24 shows LIV-1 expression in breast tumor samples and matched normal breast samples. Expression in breast tissue was determined using Clontech's Human Matched cDNA Pair Panel (BD Biosciences catalog # K1432-1, left panels) and 5 in-house matched samples obtained from Grossmont Hospital, La Mesa Calif. (right panels). RNA was isolated from each sample using TRIzol Reagent (Invitrogen, catalog # 15596026). cDNA was prepared from total RNA using Gibco BRL cDNA synthesis system (Life Technologies, catalog # 18267-021). The upper gels show LIV-1 expression; lower gels show GAPDH expression. The arrowhead on the right of the figure denotes the anticipated size of the LIV-1 PCR product. The tumor samples are as follows: (1-patient A) infiltrating ductal carcinoma, (2-patient B) infiltrating ductal carcinoma, (3-patient C) tubular adenocarcinoma, (4-patient D) infiltrating ductal carcinoma, (5-patient E) infiltrating ductal carcinoma, (6-patient A) normal, (7-patient B) normal, (8-patient C) normal, (9-patient D) normal, (10-patient E) normal, (11) negative control, (12) positive control, (13-patient G19) high grade invasive ductal carcinoma, (14-patient G17) low grade intraductal carcinoma, (15-patient X) ductal adenocarcinoma, (16-patient W) mixed ductal and lobular adenocarcinoma, (17-patient T) high grade in situ & invasive ductal carcinoma, (18-patient G19) normal, (19-patient G17) normal, (20-patient X) normal, (21-patient W) normal, (22-patient T) normal, (23) negative control, and (24) positive control. The data presented in this figure indicates that LIV-1 is expressed in all ten breast cancer samples analyzed. In 4 of the 10 samples, expression is significantly higher in the tumor tissue than in the corresponding matched normal sample.

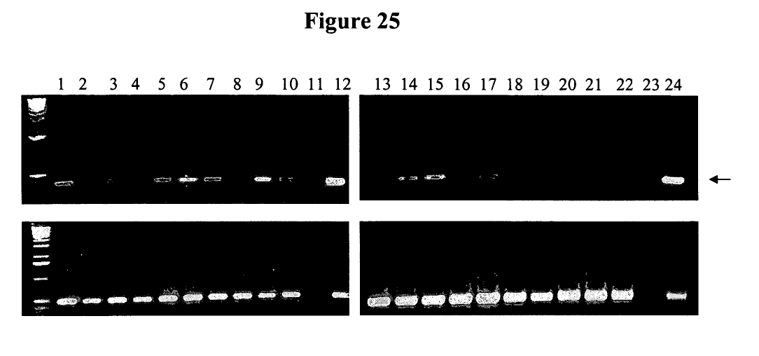

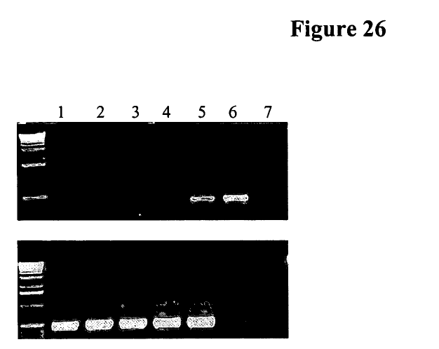

LIV-1 expression in colon tumors is shown in FIG. 25. Colon tumor samples were obtained from Grossmont Hospital in La Mesa, Calif. Colon adenocarcinoma cell line HCT116 was obtained from the American Type Culture Collection (ATCC, Rockville, Md.). RNA was isolated from each sample and cell line using Qiagen's RNeasy kit (catalog # 75162). cDNA was prepared from total RNA using Gibco BRL cDNA synthesis system (Life Technologies, catalog #18267-021). The upper panel shows LIV-1 expression, while the lower panel shows GAPDH expression. Samples are as follows: (1) grade 3 adenocarcinoma, (2) grade 2 adenocarcinoma, (3) grade 1 adenocarcinoma, (4) grade 2 adenocarcinoma, (5) colorectal cancer cell line HCT 116, (6) positive control, and (7) negative control. The data presented here indicates that LIV-1 is expressed in all 4 colon tumor samples tested.

Taken together, the data presented here indicates that LIV-1 is expressed at significant levels in multiple breast and colon tumor samples. The Gene Logic data indicates it is also overexpressed in prostate tumor samples. LIV-1 may therefore represent a pancarcinoma antigen and a suitable target for tumor therapy in any of the above mentioned indications.

### Example 8

Method of Treating Cancer

A tissue sample from a patient with cancer or suspected of having cancer is obtained. The sample may be either a biopsy sample, a pathology sample obtained after a tumor has been removed from the tissue or an archived sample previously obtained from the patient. The sample is analyzed similar to Examples 1-7.

Based on analysis of the levels of IGSF9 and/or LIV-1 in the tumor sample, a treatment regime is determined using acceptable treatment alternatives known to those skilled in the art. These may include, but are not limited to, the methods described herein, observation, mode of surgery, non-adjuvant therapies such as radiation, and adjuvant therapies such as tamoxifen or cytotoxic chemotherapy.

The invention has established that overexpression IGSF9 or LIV-1 is associated with many neoplasms. Therefore, it is significant that the present invention demonstrates that IGSF9 and LIV-1 expression levels represents an informative prognostic marker for various cancers. Expression levels of IGSF9 or LIV-1 can be determined using the antibodies, antigen binding fragments, or polynucleotides of the invention. Knowledge of the IGSF9 and LIV-1 expression levels in primary tumors at the time of diagnosis and surgical removal may therefore directly influence therapeutic decisions regarding adjuvant hormone and chemotherapies, as well as supplementary radiation therapy.

In addition to affecting the choice and utilization of currently available cancer therapies, knowledge of IGSF9 and LIV-1 expression levels may be useful for application of new cancer therapies. Therapies to restore normal levels of IGSF9 and LIV-1 expression include, but are not limited to those described above.

### Example 9

Method of Screening Compounds

The pharmaceutical industry is interested in evaluating pharmaceutically useful compounds which act as cell surface receptor agonists or antagonists. Tens of thousands of compounds per year need to be tested in an entry level or “high flux” screening protocol. Out of the thousands of compounds scrutinized, one or two will show some activity in the entry level assay. These compounds are then chosen for further development and testing. Ideally, a screening protocol would be automated to handle many samples at once, and would not use radioisotopes or other chemicals that pose safety or disposal problems. An antibody-based approach to evaluating desired or undesired drug regulation of cell surface receptor activities would provide these advantages and offer the added advantage of high selectivity.

In particular, antibodies that recognize IGSF9 or LIV-1 may be used to for screening drugs in various screening protocols. Generally, two approaches are used. Cell or tissue based approaches use an indicator cell line or tissue that is exposed to the compound to be tested. When cells are used it is thought that this approach may quickly eliminate drugs having solubility or membrane permeability problems. Protein or enzyme-based screens may use purified proteins and can identify drugs that react with IGSF9 or LIV-1 to affect intracellular signaling.

For cell or tissue based screening to identify drugs that modulate (e.g. stimulate, block, inhibit or suppress) IGSF9 or LIV-1 expression, immunohistochemistry or cytochemistry of IGSF9 or LIV-1 expression can be used to measure the effects of individual agents.

An immunohistochemistry-based method that accurately detects levels of IGSF9 or LIV-1 also has the advantage that it may be used with solid tumor explant cultures and organoid cultures, and therefore allows accurate detection of IGSF9 or LIV-1 modulating drugs in more physiologically relevant settings than those used by other methods. Furthermore, the proposed method will also be applicable to screening and monitoring the effect of drugs on IGSF9 or LIV-1 in tissues and cells in research animals and humans in vivo. Samples may be obtained by biopsy (e.g. fine needle aspiration, section) or by tissue harvesting, in the case of research animals, and then subjected to the methods of the invention.

The proposed method is highly sensitive because IGSF9 or LIV-1 expression levels, in principle, may be monitored in a single cell. For practical use, more cells may be needed, but good analytic estimates can certainly be obtained with as little as 20-100 cells.

The foregoing specification, including the specific embodiments and examples, is intended to be illustrative of the present invention and is not to be taken as limiting. Numerous other variations and modifications can be effected without departing from the true spirit and scope of the present invention. All publications, patents and patent applications cited herein are incorporated by reference in their entirety into the disclosure.

What is claimed is:

1. An isolated antibody or antigen binding fragment thereof which associates with either IGSF9 or LIV-1.

2. An isolated antibody or antigen binding fragment thereof which associates with IGSF9 between amino acids 21 to 718 as set forth in SEQ ID NO:2, between amino acids 21 to 734 as set forth in SEQ ID NO:8, the amino acid sequences as set forth in SEQ ID NOS:22-27; or with LIV-1 between amino acids 28 to 317, 373 to 417, 674 to 678 or 742 to 749, as set forth in SEQ ID NO:29.

3. The isolated antibody or antigen binding fragment of claim 2, wherein said antibody or antigen binding fragment comprises a domain deleted antibody.

4. The domain deleted antibody or antigen binding fragment thereof of claim 3, further comprising a cytotoxic agent.

5. The domain deleted antibody or antigen binding fragment thereof of claim 4, wherein said cytotoxic agent is a radionuclide.

6. The antibody or antigen binding fragment thereof of claim 1, wherein said antibody is humanized.

7. The antibody or antigen binding fragment thereof of claim 1, wherein said antibody is primatized.

8. An antibody or antigen fragment thereof which associates with IGSF9 or LIV-1, wherein said antibody or antigen binding fragment thereof inhibits one or more functions associated with IGSF9 or LIV-1.

9. A composition comprising an antibody or antigen binding fragment thereof which associates with IGSF9 or LIV-1.

10. A composition for the treatment of a neoplastic disorder comprising a domain deleted anti-IGSF9 or anti-LIV-1 antibody or antigen binding fragment thereof covalently linked to one or more bifunctional chelators.

11. The composition of claim 10, wherein said bifunctional chelator is selected from the group consisting of MX-DTPA and CHX-DTPA.

12. A method of treating a mammal exhibiting a neoplastic disorder comprising the step of administering a therapeutically effective amount of an antibody or antigen binding fragment thereof that associates with IGSF9 or LIV-1.

13. The method of claim 12 further comprising the step of administering a therapeutically effective amount of at least one chemotherapeutic agent to said mammal; wherein said chemotherapeutic agent and said antibody or antigen binding fragment thereof may be administered in any order or concurrently.

14. The method of claim 12, wherein said anti-IGSF9 or anti-LIV-1 antibody or antigen binding fragment thereof is a domain deleted antibody.

15. The method of claim 14, wherein said domain deleted antibody or antigen binding fragment thereof lacks the CH2 domain.

16. The method of claim 12, wherein said antibody or antigen binding fragment thereof is humanized.

17. The method of claim 12, wherein said antibody or antigen binding fragment thereof is associated with a cytotoxic agent.

18. The method of clam 12, wherein said antibody or antigen binding fragment thereof is administered within two weeks of said chemotherapeutic agent.

19. A vaccine for treating cancer comprising the IGSF9 or LIV-1 polypeptide or a fragment thereof and a physiologically acceptable carrier.

20. The vaccine of claim 19, wherein said polypeptide comprises amino acids 1 to 1163 or amino acids 21 to 718 of IGSF9 as set forth in SEQ ID NO:2; or amino acids 1 to 749, amino acids 28 to 317, or amino acids 373 to 417 of LIV-1 as set forth in SEQ ID NO:29.

21. The vaccine of claim 19, wherein said physiologically acceptable carrier comprises an adjuvant or an immunostimulatory agent.

22. The vaccine of claim 21, wherein said adjuvant is PROVAX™.

23. The vaccine of claim 19, wherein said polypeptide is fused to a T helper peptide.

24. A method of inducing an immune response in a patient in need of treatment or prevention of cancer, comprising administering the vaccine of claim 19 to said patient.

25. A method of diagnosing cancer by detecting overexpression of IGSF9 or LIV-1, or a fragment thereof, comprising:
e. obtaining a sample from an individual in need of diagnosis of cancer;
f. detecting expression of IGSF-9 or LIV-1, or a fragment thereof in said sample;
g. detecting expression of IGSF-9 or LIV-1, or a fragment thereof in a control sample from a normal individual, or normal tissue from the individual being diagnosed; and
h. comparing the level of expression of IGSF-9 or LIV-1 to that obtained in the control sample, wherein said comparison results in diagnosing cancer.

26. The method of claim 25, wherein said IGSF9 fragment comprises exons 5-10.

27. The method of claim 25, wherein said overexpression is detected by nucleic acid amplification or hybridization.

28. The method of claim 25, wherein said overexpression is detected using an antibody to IGSF9 or LIV-1, or an antigen binding fragment thereof.

29. A method for determining the prognosis of an individual receiving a cancer treatment comprising:
e. obtaining a sample from said individual in need of prognosis of cancer treatment;
f. detecting expression of IGSF9 or LIV-1, or a fragment thereof in said sample;
g. detecting expression of IGSF9 or LIV-1, or a fragment thereof in a control sample from a normal individual, or normal tissue from the individual being diagnosed; and
h. comparing the level of expression of IGSF9 or LIV-1 to that obtained in the control sample, wherein said comparison results in a cancer prognosis.

30. The method of claim 29, wherein said IGSF9 fragment comprises exons 5-10.

31. A vaccine comprising as an active ingredient, an anti-idiotypic antibody that immunologically mimics the IGSF9 or LIV-1 antigens or fragments thereof.

32. A kit comprising the composition of claim 9 together with instructions for use thereof to treat or detect cancer.

33. A method of treating a neoplastic disorder in a mammal wherein neoplastic cells express the IGSF9 or LIV-1 antigens, comprising administering to said mammal a composition comprising a pharmaceutically effective amount of an antibody to IGSF9 or LIV-1, or an antigen binding fragment thereof.

34. A vaccine comprising a pharmaceutically acceptable carrier and an anti-tumor immune-response-inducing effective amount of an immunogenic preparation comprising IGSF9 or LIV-1, wherein said immunogenic preparation is capable of inducing an anti-tumor immune response.

35. An antisense nucleic acid up to 50 nucleotides in length comprising at least an 8 nucleotide portion of IGSF9 or LIV-1 which inhibits the expression of IGSF9 or LIV-1.

36. The nucleic acid of claim 35, wherein the antisense oligonucleotide comprises at least one modified internucleotide linkage.

37. A method of inhibiting the expression of IGSF9 or LIV-1 in cells or tissues comprising contacting said cells or tissues with the nucleic acid of claim 34 so that expression of IGSF9 or LIV-1 is inhibited.

38. An isolated nucleic acid selected from the group consisting of:
SEQ ID NO:3;
SEQ ID NO:5;
SEQ ID NO:12;
SEQ ID NO:13;
SEQ ID NO:14;
SEQ ID NO:15;
SEQ ID NO:16;
SEQ ID NO:17;
SEQ ID NO:18;
SEQ ID NO:19;
SEQ ID NO:20; and
SEQ ID NO:21.

39. A vector comprising the nucleic acid of claim 38.

40. A host cell comprising the nucleic acid of claim 38.

41. An isolated polypeptide selected from the group consisting of:
SEQ ID NO:4;
SEQ ID NO:6;
SEQ ID NO:22;
SEQ ID NO:23;
SEQ ID NO:24;
SEQ ID NO:25;
SEQ ID NO:26; and
SEQ ID NO:27.

42. A composition comprising the polypeptide of claim 41.

43. A vaccine for treating cancer comprising the polypeptide of claim 41 and a physiologically acceptable carrier.
